SEQUENCE LISTING (31 sequences)

SEQ ID NO: 1 - DNA, 3174 bp, synthetic construct
  source 1..3174: mol_type = other DNA
gacggatcgggagatctcccgatgcgggtgccagggcgtgcccttgggctccccgggcgcgtactcctcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgttttaaaagaaaaggggggattggggggtacagtgcaggggaaagaatagtagacataatagcaacagacatacaaactaaagaattacaaaaacaaattacaaaaattcaaaattttaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggagacccaagctggctagcgtttaaacttaagcttggtaccgagctcggatccgccgccaccatggccttaccagtgaccgccttgctcctgccgctggccttgctgctccacgccgccaggccggatatccagatgacccagacaacctccagcctgtccgcctccctgggcgatagagtgacaatctcctgcagggcctcccaggatatcagcaagtacctgaactggtaccagcagaagcccgacggcacagtgaagctgctgatctaccacaccagcaggctgcactccggcgtgccctccaggttcagcggctccggaagcggcaccgactacagcctgaccatcagcaatctggagcaggaggatatcgccacctacttttgccagcagggcaataccctgccctacaccttcggcggcggcaccaagctggagatcaccggcagcacaagcggctccggcaagcctggctccggcgaaggaagcacaaagggcgaggtgaagctgcaggagagcggccctggcctggtggctcctagccagtccctgtccgtgacctgtaccgtgtccggcgtgtccctgcctgactacggcgtgagctggatcaggcagccccccagaaagggcctggagtggctgggcgtgatctggggctccgagaccacatactacaattccgccctgaagagcaggctgaccatcatcaaggataatagcaagtcccaggtgtttctgaagatgaactccctgcagaccgacgacaccgccatctactactgcgccaagcactactactacggcggcagctacgccatggactactggggccagggcaccagcgtgaccgtgtcctccggaaaacctatccctaatccactgcttggactcgatagtactaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaatctagagggcccgtttaaacccgctgatcagcctcgaaaaaaaaaaaaaaaaaaaaaaaaaaaagaagacggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccggggataacgcaggaaagaacatgtgagcaaaaggccagcaaaaggccaggaaccgtaaaaaggccgcgttgctggcgtt

SEQ ID NO: 2 - DNA, 63 bp, synthetic construct
  source 1..63: mol_type = other DNA
atggccttaccagtgaccgccttgctcctgccgctggccttgctgctccacgccgccaggccg

SEQ ID NO: 3 - DNA, 321 bp, synthetic construct
  source 1..321: mol_type = other DNA
gatatccagatgacccagacaacctccagcctgtccgcctccctgggcgatagagtgacaatctcctgcagggcctcccaggatatcagcaagtacctgaactggtaccagcagaagcccgacggcacagtgaagctgctgatctaccacaccagcaggctgcactccggcgtgccctccaggttcagcggctccggaagcggcaccgactacagcctgaccatcagcaatctggagcaggaggatatcgccacctacttttgccagcagggcaataccctgccctacaccttcggcggcggcaccaagctggagatcacc

SEQ ID NO: 4 - DNA, 54 bp, synthetic construct
  source 1..54: mol_type = other DNA
ggcagcacaagcggctccggcaagcctggctccggcgaaggaagcacaaagggc

SEQ ID NO: 5 - DNA, 360 bp, synthetic construct
  source 1..360: mol_type = other DNA
gaggtgaagctgcaggagagcggccctggcctggtggctcctagccagtccctgtccgtgacctgtaccgtgtccggcgtgtccctgcctgactacggcgtgagctggatcaggcagccccccagaaagggcctggagtggctgggcgtgatctggggctccgagaccacatactacaattccgccctgaagagcaggctgaccatcatcaaggataatagcaagtcccaggtgtttctgaagatgaactccctgcagaccgacgacaccgccatctactactgcgccaagcactactactacggcggcagctacgccatggactactggggccagggcaccagcgtgaccgtgtcctcc

SEQ ID NO: 6 - DNA, 135 bp, synthetic construct
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 7 - DNA, 81 bp, synthetic construct
  source 1..81: mol_type = other DNA
ttctgggtgctggtcgttgtgggcggcgtgctggcctgctacagcctgctggtgacagtggccttcatcatcttttgggtg

SEQ ID NO: 8 - DNA, 123 bp, synthetic construct
  source 1..123: mol_type = other DNA
aggagcaagcggagcagactgctgcacagcgactacatgaacatgaccccccggaggcctggccccacccggaagcactaccagccctacgcccctcccagggatttcgccgcctaccggagc

SEQ ID NO: 9 - DNA, 126 bp, synthetic construct
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 10 - DNA, 336 bp, synthetic construct
  source 1..336: mol_type = other DNA
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 11 - DNA, 10818 bp, synthetic construct
  source 1..10818: mol_type = other DNA
ccttattttaactaagcttaaaaaaatggggccgtgccccaactggggtaacctttgagttctctcagttgggggactagggacaggattggtgacagaaaagccccatcgcaccgactcggtgccactttttcaagttgataacggactagccttattttaacttgctatttctagctctaaaacagtggccccactgtggggtgcggtgatgctgaattacagaactcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttggtaccggatccagtcgacgccatttgtctgcagaattggcgcacgcgctaaaaacggactagccttattttaacttgctatttctagctctaaaacaaaaaagcaccgactcggtgccactttttcaagttgataacggactagccttattttaacttgctatttctagctctaaaactgccccaactggggtaacctcggtgatggaggtgctgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgcgagcaaaatttaagctacaacaaggcaaggcttgaccgacaattgcatgaagaatctgcttagggttaggcgttttgcgctgcttcgcgatgtacgggccagatatacgcgtttttaaaagaaaaggggggattggggggtacagtgcaggggaaagaatagtagacataatagcaacagacatacaaactaaagaattacaaaaacaaattacaaaaattcaaaattttctagtccgtttttagggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaatttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtctactctcagtacaatctgctctgatgccgcatagttaagccagtatctgctccctgcttgtgtgttggaggtcgctgagtagtg

SEQ ID NO: 12 - DNA, 84 bp, synthetic construct
  source 1..84: mol_type = other DNA
tggggccgtgccccaactggggtaacctttgagttctctcagttgggggactagggacaggattggtgacagaaaagccccatc

SEQ ID NO: 13 - DNA, 76 bp, synthetic construct
  source 1..76: mol_type = other DNA
gcaccgactcggtgccactttttcaagttgataacggactagccttattttaacttgctatttctagctctaaaac

SEQ ID NO: 14 - DNA, 25 bp, synthetic construct
  source 1..25: mol_type = other DNA
agtggccccactgtggggtgcggtg

SEQ ID NO: 15 - DNA, 76 bp, synthetic construct; identical to SEQ ID NO: 13
  source 1..76: mol_type = other DNA

SEQ ID NO: 16 - DNA, 25 bp, synthetic construct
  source 1..25: mol_type = other DNA
tgccccaactggggtaacctcggtg

SEQ ID NO: 17 - DNA, 118 bp, synthetic construct
  source 1..118: mol_type = other DNA
ttttaaaagaaaaggggggattggggggtacagtgcaggggaaagaatagtagacataatagcaacagacatacaaactaaagaattacaaaaacaaattacaaaaattcaaaatttt

SEQ ID NO: 18 - DNA, 4128 bp, synthetic construct
  source 1..4128: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctct

SEQ ID NO: 19 - DNA, 5053 bp, synthetic construct
  source 1..5053: mol_type = other DNA
acggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcgctagaaataattttgtttaactttaagaaggagatatacatatggacacgtacgcgggtgcttacgaccgtcagtcgcgcgagcgcgaaaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtagcgagacacccggatcgcgccaaaaaaaaaaaaaaaaaaaaaaaaaaaaaaaccggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccctgtggaattgtctgtataccgtcgacctctagctagagcttggcgtaatcatggtcatagctgtttcctgtgggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 20 - DNA, 1818 bp, synthetic construct
  source 1..1818: mol_type = other DNA
atggacacgtacgcgggtgcttacgaccgtcagtcgcgcgagcgcgaaaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtag

SEQ ID NO: 21 - protein, 671 aa, synthetic construct
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 22 - protein, 1384 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSDLSQLGGD

SEQ ID NO: 23 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 24 - DNA, 34 bp, synthetic construct
  source 1..34: mol_type = other DNA
gtgccagggcgtgcccttgggctccccgggcgcg

SEQ ID NO: 25 - DNA, 3174 bp, synthetic construct; identical to SEQ ID NO: 1
  source 1..3174: mol_type = other DNA

SEQ ID NO: 26 - DNA, 10818 bp, synthetic construct
  source 1..10818: mol_type = other DNA
ccttattttaactaagcttaaaaaaattcctgcccgaccttggtaccggatccagtcgacgccatttgtctgcagaattggcgcacgcgctaaaaacggactagccttattttaacttgctatttctagctctaaaacaaaaaagcaccgactcggtgccactttttcaagttgataacggactagccttattttaacttgctatttctagctctaaaactgccccaactggggtaacctcggtgatggaggtgctgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgcgagcaaaatttaagctacaacaaggcaaggcttgaccgacaattgcatgaagaatctgcttagggttaggcgttttgcgctgcttcgcgatgtacgggccagatatacgcgtttttaaaagaaaaggggggattggggggtacagtgcaggggaaagaatagtagacataatagcaacagacatacaaactaaagaattacaaaaacaaattacaaaaattcaaaattttctagtccgtttttagggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaatttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagttctgtaattcagcatcaccgcaccccacagtggggccactgttttagagctagaaatagcaagttaaaataaggctagtccgttatcaacttgaaaaagtggcaccgagtcggtgcgatggggcttttctgtcaccaatcctgtccctagtcccccaactgagagaactcaaaggttaccccagttggggcacggccccaacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtctactctcagtacaatctgctctgatgccgcatagttaagccagtatctgctccctgcttgtgtgttggaggtcgctgagtagtg

SEQ ID NO: 27 - DNA, 5053 bp, synthetic construct; identical to SEQ ID NO: 19
  source 1..5053: mol_type = other DNA

SEQ ID NO: 28 - RNA, 20 nt, synthetic construct
  source 1..20: mol_type = other RNA
agagtctctcagctggtaca

SEQ ID NO: 29 - RNA, 20 nt, synthetic construct
  source 1..20: mol_type = other RNA
gagtagcgcgagcacagcta

SEQ ID NO: 30 - RNA, 20 nt, synthetic construct
  source 1..20: mol_type = other RNA
gacagcggcacctacctctg

SEQ ID NO: 31 - RNA, 20 nt, synthetic construct
  source 1..20: mol_type = other RNA
cagcctcctggttatggtac

(19) United States
(12) Patent Application Publication
(10) Pub. No.: US 2026/0234558 A1
(43) Pub. Date: Aug. 13, 2026

(54) Methods and Systems for Rapid Engineering of Resting Cells

(71) Applicant: CellFE, Inc., Alameda, CA (US)

(72) Inventors: Chih-Wen Ni, Alameda, CA (US); Ailin Goff, Alameda, CA (US)

(21) Appl. No.: 19/377,671

(22) Filed: Nov. 3, 2025

Related U.S. Application Data
(63) Continuation of application No. PCT/US2024/028038, filed on May 6, 2024.
(60) Provisional application No. 63/500,489, filed on May 5, 2023.

(51) Int. Cl. C12N 5/0783; C07K 14/705; C12N 5/074; C12N 5/079; C12N 9/12; C12N 9/22; C12N 15/10; C12N 15/113; C12N 15/52; C12N 15/85

(52) U.S. Cl. CPC C12N 5/0636; C07K 14/70503; C12N 5/0607; C12N 5/0618; C12N 5/0646; C12N 9/1241; C12N 9/226; C12N 15/102; C12N 15/113; C12N 15/52; C12N 15/85; C12N 2310/20; C12N 2510/00

(57) ABSTRACT

Non-viral methods and systems for rapidly transfecting resting cells with exogenous material are described. The methods and systems may be used to rapidly transfect cells with multiple components and can be used to manufacture modified immune cells such as resting T-cells.

### CROSS REFERENCE TO RELATED APPLICATIONS

The present application is a continuation of PCT/US2024/028038, filed on 2024 May 6, which claims the benefit of U.S. Provisional Application No. 63/500,489, entitled, “METHODS AND SYSTEMS FOR RAPID ENGINEERING OF RESTING CELLS” filed 2023 May 5, the entire contents of which is hereby incorporated by reference.

### INCORPORATION OF SEQUENCE LISTING

The present application contains a Sequence Listing which has been submitted in .XML format via Patent Center and is hereby incorporated by reference in its entirety. Said WIPO Sequence Listing was created on 2026 Mar. 13, is named 120420-797284_Sequence listing, and is 76,597 bytes in size.

### FIELD

The present disclosure relates generally to cell genetic engineering methods and systems.

### BACKGROUND

Engineering of immune cells is the basis of relatively new advances in immunotherapies for treating patients with cancer and other diseases. CAR-T cell therapy is an example of a newer, very promising therapy, which relies on the genetic modification of T-cells using some form of gene delivery, so that the cells are modified to express chimeric antigen receptors (CARs).

Gene delivery can use viral or non-viral means. Viral vectors however carry increased risks of immunogenicity and pathogenicity, and viral transduction of multiple different nucleic acid payloads to achieve complex or multiple edits is time-consuming and not readily scalable. Unlike viral delivery, known non-viral knock-in edits rely on endogenous homology directed repair (HDR) machinery which is operational only in actively dividing (i.e., not in resting) cells. This requirement creates a bottleneck in cell manufacturing, e.g., for T-cell manufacturing, because the cells must be activated and dividing to be successfully transfected, and then they must be expanded to attain useful numbers. As a consequence, the entire process typically takes several days to weeks and the delay presents multiple challenges, including: effector differentiation, exhaustion, and apoptosis which results in the failure of patients' response; vast investment of time, materials and labor required to create the cells; and limited benefits or even detriment for patients with rapidly progressing disease and in resource-poor healthcare environments.

A significant need exists for cell engineering methods that do not rely on HDR machinery and can be performed on resting cells such as resting T-cells, which preserve cell expansion capacity, cell potency, delay cell differentiation, prevent potential exhaustion of cells, and allow for multiple

### BRIEF SUMMARY

Various aspects of the disclosure are listed below. It will be understood that the aspects listed below may be combined not only as listed, but in other suitable combinations in accordance with the spirit and scope of the invention.

Provided herein is a method for modifying a cell, which in one aspect comprises: contacting the cell with a first set of components of a genome editing system, the first set of components comprising a guide RNA having a nucleotide sequence capable of hybridizing to a target location, a pegRNA having an attP attachment site, and a nickase or a nucleic acid having a nucleotide sequence encoding the nickase; and contacting the cell with a second set and a third set of components of a genome editing system, the second set comprising a serine-integrase (PhiC31) or a nucleic acid having a nucleotide sequence encoding a serine-integrase (PhiC31), and the third set comprising a nucleotide sequence encoding a nucleic acid sequence of interest and an attB site docking site.

In certain instances, contacting the cell with the second set of components and third set of components may be performed as a co-transfection event.

In certain instances, the method for modifying the cell effects an insertion of the nucleic acid sequence of interest into the genome of the cell.

In certain instances, the nucleic acid sequence of interest is inserted without occurrence of homologous recombination.

In certain instances, contacting the cell with the first set, second set and third set of genomic editing components delivers the components to the cell in the absence of, i.e., without the use of a viral vector.

In certain instances, contacting the cell with the first, second and third sets of genome editing components comprises combining the cell in fluid medium with the first, second and third sets of genome editing components and passing the fluid medium through at least one microfluidic transfection device.

In certain instances, a first vector may comprise the first set of genomic editing components, a second vector comprises the second set of genomic editing components, and a third vector comprises the third set of genome editing components.

In certain instances, the first, second and third vectors may be delivered to the cell by passing the cell in fluid medium through one or more microfluidic transfection devices.

In certain instances, the nickase may comprise a Cas9 nickase.

In certain instances, the nickase may comprise nCas9 having a nucleic acid sequence having at least 70%, 80%, 90% or 100% sequence identity with SEQ ID NO: 18.

In certain instances, the nickase may comprise an nCas9-M-MLV fusion protein with M-MLV having an amino acid sequence having at least 70%, 80%, 90% or 100% sequence identity with SEQ ID NO: 21.

In certain instances, the cell may be selected from an immune cell, a neural cell and a stem cell, wherein optionally the cell is

In certain instances, the cell may be a resting immune cell.

In certain instances, the cell may be a resting T cell.

In certain instances, the cell may be a resting NK cell.

In certain instances, the cell may be a naïve stem cell, or a pluripotent stem cell.

In certain instances, the insertion may be a nucleic acid sequence encoding a chimeric antigen receptor (CAR).

In certain instances, the cell may be a T cell or an NK cell and the method may further comprise maintaining the cell for a time and under conditions sufficient for the cell to form a CAR-T cell or a CAR-NK cell.

In certain instances, forming a CAR-T cell or a CAR-NK cell may not comprise use of a viral vector.

In certain instances, the first vector may comprise a nickase comprising a nucleotide sequence having at least 70%, 80%, 90%, or 100% sequence identity with SEQ ID NO: 18. (nCas9)

In certain instances, the nucleotide sequence encoding serine-integrase encodes PhiC31 and may have a nucleotide sequence having at least 70%, 80%, 90%, or 100% sequence identity with SEQ ID NO: 20.

In certain instances, the first vector may comprise the sequence of SEQ ID NO: 1.

In certain instances, the second vector may comprise the sequence of SEQ ID NO:11.

In certain instances, the third vector may comprise the sequence of SEQ ID NO:19.

In certain instances, the first, second and third sets of components of the genomic editing system genomic may be introduced to the cell within a period of no more than 144 hours, 120 hours, 96 hours, 72 hours, 60 hours, 48 hours, 36 hours, 24 hours, 16 hours, 8 hours, 6 hours, or 1 hour.

Further provided herein is a non-viral system for modifying a cell, the system may comprise: (i) a first genetic construct comprising a guide RNA nucleotide sequence capable of hybridizing to a target site, a pegRNA having an attP site, and a nickase or a nucleotide sequence encoding the nickase; (ii) a serine-integrase (PhiC31) or a second genetic construct comprising a nucleotide sequence encoding the serine-integrase (PhiC31); and (iii) a third genetic construct comprising a nucleotide sequence encoding the nucleic acid sequence of interest and an attB sequence.

In certain instances, the first, second and third genetic constructs may be non-viral.

In certain instances, the first, second and third genetic constructs each respectively may comprise a first plasmid vector, a second plasmid vector and a third plasmid vector.

In certain instances, the cell may be selected from an immune cell, a neural cell and a stem cell.

In certain instances, the cell may be selected from a resting immune cell, optionally a resting T cell or a resting NK cell.

In certain instances, the nucleic acid of interest may comprise a sequence encoding a chimeric antigen receptor (CAR) molecule.

In certain instances, the nickase may comprise the nucleotide sequence of SEQ ID NO: 18. (nCas9)

In certain instances, the nucleotide sequence encoding a nickase may encode an nCas9-M-MLV fusion protein with an M-MLV having the sequence of SEQ ID NO: 21.

In certain instances, the nucleotide sequence encoding serine-integrase may encode PhiC31 having the sequence of SEQ ID NO: 20.

In certain instances, the first vector may comprise the sequence of SEQ ID NO: 1.

In certain instances, the second vector may comprise the sequence of SEQ ID NO: 11.

In certain instances, the third vector may comprise the sequence of SEQ ID NO: 19.

Further provided herein is a cell comprising the system of the preceding instances.

In certain instances, the cell may be disposed in a microfluidic device.

Further provided here is a cell or a population of cells in a fluid medium in combination with any one or more of (i) a first genetic construct comprising a guide RNA nucleotide sequence capable of hybridizing to a target site, a pegRNA having an attP attachment site, and a nickase or a nucleotide sequence encoding the nickase; (ii) a serine-integrase (PhiC31) or a second genetic construct comprising a nucleotide sequence encoding the serine-integrase (PhiC31); and (iii) a third genetic construct comprising a nucleotide sequence encoding the nucleic acid sequence of interest and an attB sequence.

In certain instances, the cell may be disposed in a microfluidic device.

In certain instances, the cell or cells may not be purified or isolated from a donated cell population.

In certain instances, the cell or cells may comprise a resting immune cell or cells.

In certain instances, the cell or cells may comprise a CAR-T or CAR-NK cell or cells.

In certain instances, provided herein is a population of cells modified by the methods of the instances above.

In certain instances, the cell or cells may not bet purified or isolated from a donated cell population.

In certain instances, the method may further comprise before combining the cell with the first, second or third set of genomic editing components, maintaining the cell for a period of time in a culture medium comprising at least one of interleukin (IL)-2, IL-7 and IL-15, or any combination thereof.

In certain instances, the period of time may be at least about 2 days for the cell to enter a state where the cell is ready for successful transfection.

In certain instances, the method may be performed on a population of cells wherein the cells are modified within about 5-6 days.

In certain examples, the cell may remain cultured in the culture medium for the entirety of the method.

In certain instances, the method may further comprise determining that the cells are modified by detecting the presence of an exogenous nucleic acid or exogenous polypeptide sequence in the cell.

In certain instances, the method may further comprise determining that cells are modified by detecting the insertion of an exogenous nucleic acid sequence by performing genomic nucleic acid sequencing or PCR analysis on the cell contents.

In certain instances, the method may further comprise subjecting the cells to flow cytometry or imaging acquisition to detect a reporter gene expression indicative of exogenous nucleic acid expression.

In certain instances, the method may further comprise subjecting the cells to flow cytometry analysis or imaging acquisition and an antibody or fluorophore tag to detect the presence of an exogenous polypeptide sequence.

The disclosure further encompasses a method of editing the genome of a cell or of a cell population, the method comprising: wherein the cell or cell population in a fluid medium of is disposed in a microfluidic device, contacting the cell or cells in series with a first set of components of a genome editing system comprising guide RNA nucleotide sequence capable of hybridizing to a first target site, and a nickase or a nucleotide sequence encoding the nickase; and with at least a second set of components of a genome editing system comprising a second guide RNA nucleotide sequence capable of hybridizing to a second target site, and a nickase or a nucleotide sequence encoding the nickase.

In certain instances, the method further comprises an holding period of 1 hour to 48 hours, wherein the cell or cell population is held in culture, between contacting the cells or cells with the first and the second set of genome editing components.

In certain instances, the cell is selected from an immune cell, a neural cell and a stem cell. In one aspect, the cell is selected from a resting immune cell, optionally a resting T cell or a resting NK cell.

Further encompassed herein, is the cell or cell population, comprising a resting immune cell or cells disclosed herein. In certain instances, the cells have greater than 5% transfection efficiency, about 70% cell viability, preserved expansion capacity, preserved stemness with about 50% Tscm population, or any combinations thereof.

In certain instances, the method disclosed herein further comprises priming the resting T cells with a at least one cytokine. In certain aspects, the priming the cells comprises contacting the cell or cell population with the at least one cytokine for about 24 hours to about 72 hours. In one aspect, the cells demonstrate about 80% cell viability, preserved expansion capacity, preserved stemness with about 50% Tscm population, or any combination thereof.

Other aspects and iterations of the invention are described more thoroughly below.

### BRIEF DESCRIPTION OF THE DRAWINGS

FIG. 1 is a flow chart of a non-viral transfection method showing expected timelines of the two-step transfection process.

FIG. 2 is a flow chart of a non-viral transfection method illustrating a two-step delivery of three sets of genomic editing components for a knock-in edit of a resting cell at a target location.

FIG. 3 is a schematic map of a plasmid vector design for a first transfection event to deliver CRISPR prime editing components and an attP attachment sequence for the knock-in edit of a resting cell at a target location as shown in FIG. 2.

FIG. 4 is a schematic map of a plasmid vector design for a second transfection event, to achieve a serine-integrase (PhiC31)-mediated recombination between the attB and attP site for a knock-in edit of a resting cell.

FIGS. 5-18 describe various aspects of a sequential microfluidic processing system that can be used in various aspects of the disclosure, as follows:

FIG. 5 is a microfluidic sequential processing system in one example.

FIG. 6 is a flow chart of a microfluidic sequential process in one example.

FIG. 7 is a side view of a microfluidic transfection device.

FIG. 8 is a microfluidic sequential processing system in one example.

FIG. 9 is a flow chart of a microfluidic sequential process in one example.

FIG. 10 is a microfluidic sequential processing system in one example.

FIG. 11 is a flow chart of a microfluidic sequential process in one example.

FIG. 12 is a microfluidic sequential processing system in one example.

FIG. 13 is a flow chart of a microfluidic sequential process in one example.

FIG. 14A is a microfluidic sequential processing system in one example.

FIGS. 14B-C are examples of microfluidic transfection devices.

FIG. 15 is a flow chart of a microfluidic sequential process in one example.

FIG. 16A is a top view of a microfluidic sequential processing system in one example.

FIG. 16B is a side view of a microfluidic sequential processing system in one example.

FIG. 16C is a microfluidic sequential processing system in one example.

FIG. 16D is a side view of a microfluidic sequential processing system in one example.

FIG. 16E is a top view of a microfluidic sequential processing system in one example.

FIG. 17 is a flow chart of a microfluidic sequential process in one example.

FIG. 18 is a microfluidic sequential processing system in one example.

FIG. 19 is a flow chart of a microfluidic sequential process in one example.

FIG. 20 is a top view of a microfluidic transfection device in one example.

FIG. 21 is a schematic map of a first plasmid vector for a first transfection event to deliver CRISPR prime editing components and an attP attachment sequence for the knock-in edit of a resting cell at a target location as shown in FIG. 2.

FIGS. 22 and 23 are schematic maps of a second and a third plasmid vector for a second transfection event, to achieve a serine-integrase (PhiC31)-mediated recombination between the attB and attP site for a knock-in edit of a resting cell.

FIG. 24A is a bar graph of percentage of raw viability at D0 and D5 post-transfection with 48 hours of T cell activation.

FIG. 24B is a bar graph of percentage of raw viability before and after sequential editing.

FIG. 24C is a bar graph of percentage of raw viability at D0 and D5 post-transfection in T-cells sequentially edited with 24 hours of T cell activation.

FIG. 24D is a bar graph of percentage of raw viability at D0 and D5 post-transfection in T-cells, 6 hours after T cell activation in cells with single delivery, co-delivery, sequential delivery, and No Device (ND) controls.

FIG. 25A is a bar graph of percentage of knock-outs in T-cells with or without sequential editing with 48 hours of T cell activation.

FIG. 25B is a bar graph of percentage of knock-outs in T-cells with or without sequential editing with 24 hours of T cell activation.

FIG. 25C is a bar graph of percentage of knock-outs in T-cells 6 hours after T cell activation in cells with single delivery, co-delivery, sequential delivery, and No Device (ND) controls.

FIG. 26 is a bar graph of relative frequency of translocation in T cells with sequential delivery or co-delivery.

FIG. 27 is a bar graph showing population of T cell subsets after priming with cytokine at day 0 and day 3 compared to control culture without cytokine.

FIG. 28A is a bar graph of percentage of GFP expression in three different primed resting T cell lines.

FIG. 28B is a bar graph of percentage viability with or without transfection in three different primed resting T cell lines.

FIG. 28C is a bar graph of total live cells with or without transfection in three different primed resting T cell lines.

FIG. 29 is a series of bar graphs of percentage of expression and viability in cytokine primed resting T cells with TCR, B2M or CD52 knock out.

FIG. 30A is a bar graph of percentage viability of primed resting T cells after each event.

FIG. 30B is a bar graph of live cell number of primed resting T cells after each event.

FIG. 30C is a series of bar graphs showing transfection efficiency (right panel) and viability (left panel) of primed resting T cells with multiplex or sequential knock outs.

FIG. 31A is a bar graph of total live cells pre and post thaw engineered resting T cells.

FIG. 31B is a bar graph of percentage viability of post thaw engineered resting T cells from Day 0 to Day 7.

FIG. 31C is a line graph of total live cells post thaw over day 0 to day 7.

FIG. 31D is a density plot showing T cell activation in cells with B2M knock out and control cells, at 48 hours.

FIG. 32A is a schematic map of a plasmid to deliver CRISPR prime editing components and an attP attachment sequence.

FIG. 32B is a schematic map of plasmid to deliver Cas9.

FIG. 32C is a schematic map of plasmid to deliver PhiC31 integrase.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, “substantially cylindrical” means that the object resembles a cylinder but can have one or more deviations from a true cylinder.

The terms “attached” and “connected” are used interchangeably in this disclosure. The terms “attached” and “connected” mean to be connected.

The terms “comprising,” “including” and “having” are used interchangeably in this disclosure. The terms “comprising,” “including” and “having” mean to include, but not necessarily be limited to the things so described.

The terms “payload reservoir” and “payload injector” are used interchangeably in this disclosure.

Existing cell engineering methods such as those routinely used for T-cell manufacturing for CAR-T cell production are limited in application to activated cells because the cell's proliferated status and engagement of the HDR mechanism is important for successful knock-in. This limitation results in a long turnover time before cells are effectively modified, during which the activated/expanded T-cell may lose their potency and elicit poorer therapeutic response in patients. Accordingly, disclosed herein are new methods and systems for cell genome modification which can be used to modify resting (i.e., inactivated) cells including resting immune cells such as resting T-cells or natural killer (NK) cells, to more rapidly achieve desired cell genome modifications. The disclosed methods and systems can be delivered to cells using non-viral methods, such as but not limited to microfluidic transfection devices and methods that are capable of delivering sequential payloads, as described further below.

In one aspect, the methods and systems disclosed herein do not include or rely upon viral components for transfection. It should however be understood that the genetic constructs and systems described herein can be adapted to cell transfection methods other than the microfluidic methods and systems described in detail herein, as also described in part in METHODS AND SYSTEMS FOR INTRACELLULAR DELIVERY, WIPO Patent Application WO/2020/117856 (PCT/US2019/064310, filed Dec. 3, 2019, the entire contents of which are incorporated by reference).

Non-viral transfection methods avoid the known limitations of viral transfection. By way of non-limiting example, a microfluidic transfection system as described herein can be used for transfection. In other non-limiting examples, chemical transfection or electroporation transfection can be used.

### (i) Methods and Systems

In one aspect, a method for modifying a cell comprises: (a) contacting the cell with a first set of components of a genome editing system, which first set of components comprises a guide RNA (gRNA) having a nucleotide sequence capable of hybridizing to a target location, a pegRNA having an attP attachment site, and a nickase or a nucleic acid having a nucleotide sequence encoding the nickase; and (b) contacting the cell with a second set and a third set of components of a genome editing system, the second set of components comprising a serine-integrase (PhiC31) or a nucleic acid having a nucleotide sequence encoding a serine-integrase (PhiC31), and the third set of components comprising a nucleotide sequence encoding a nucleic acid sequence of interest and an attB site docking site. In one aspect, the cell is contacted with the second set of components and third set of components simultaneously so that the second set and third set of components are delivered to the cell in a single, co-transfection event.

While the methods and systems described herein can be adapted to modify cells with knock-ins of exogenous sequences, knock-outs of genomic sequences, or precise base edits of genomic sequences, in an exemplary aspect, the modification is a knock-in, i.e., an insertion of the nucleic acid sequence of interest into the genome of the cell. The methods and systems described herein provide for insertion of the nucleic acid sequence of interest in the absence of homologous recombination or reliance on the cell's homology-directed repair (HDR) mechanism.

The first set of genomic editing components comprises the gRNA which has a nucleotide sequence capable of hybridizing to a target location, the pegRNA having an attP attachment site, and a nickase or a nucleic acid having a nucleotide sequence encoding the nickase. The first set of genomic editing components thus includes primary components of a CRISPR-based genome editing system. In a CRISPR-based genome editing system, the gRNA is or comprises an RNA sequence that recognizes the target DNA location and guides the nickase to the target location to achieve the editing. The gRNA comprises crispr RNA (crRNA), which is nucleotide sequence of approximately 17-22, or 18-20 nucleotides in length which is complementary to the target DNA, and a tracr RNA, which serves as a binding scaffold for the nickase. A CRISPR-based prime editing system comprises a fusion protein, consisting of a modified, catalytically impaired endonuclease or nickase, such as a Cas9 nickase fused to an engineered reverse transcriptase enzyme, and a prime editing guide RNA (pegRNA), capable of identifying the target site and providing the new genetic information to replace the target DNA nucleotides. The prime editing system can produce targeted insertions, deletions, and base changes without the need to induce double strand breaks (DSBs), or the need for a donor template. In an example, the CRISPR-based prime editing system may be a CRISPR prime editing 3.0 system, where the pegRNA and sgRNA use the same nCas9, and the sgRNA targets the core sequence created by pegRNA to improve the final success rate.

The nickase is a non-specific endonuclease. As used herein, a “nickase” refers to a programmable nuclease that has been modified so that it cleaves only a single strand of a DNA substrate, rather than a nuclease which cleaves both strands. Nickases create site-specific “nicks” in the single strand of a DNA substrate and reduce off-target effects. Nickases can be any suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc. For example, Cas9 nickases are typically composed a mutant form of the Cas9 nuclease in which a single catalytic amino acid residue is changed. In a CRISPR prime editing system, the nickase may take the form of a fusion protein, such as for example the illustrative nCas9-M-MLV fusion protein. The nickase, along with expression modules of the pegRNA provide the components to perform CRISPR prime editing to insert the attP site at the desired locus. Attachment sites attP and attB are substrates for the serine integrase used in step (b), which mediates site-specific recombination to generate attachment sites attL and attR. In some aspects, Cas9 nickase comprise SpCas9, SaCas9, SpCas9-HF, eSPcas9, xCas9 or a cpf1.

In one aspect of the methods and systems, the nickase comprises or consists of an amino acid having the sequence of SEQ ID NO: 22, or an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 22. The nickase may be encoded by a nucleotide sequence comprising or consisting of the sequence of SEQ ID NO.18, or a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO:18. The nickase may comprise or consist of a fusion protein acid having an M-MLV with the amino acid sequence of SEQ ID NO: 21, or an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 21.

In another aspect of the methods and systems, the serine-integrase comprises or consists of an amino acid having the sequence of SEQ ID NO: 23, or an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 23. The serine-integrase may be encoded by a nucleotide sequence comprising or consisting of the sequence of SEQ ID NO: 20, or a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 20.

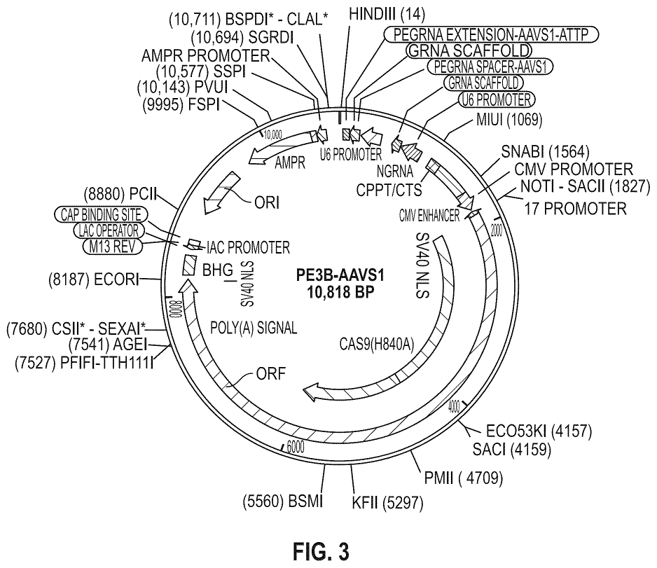

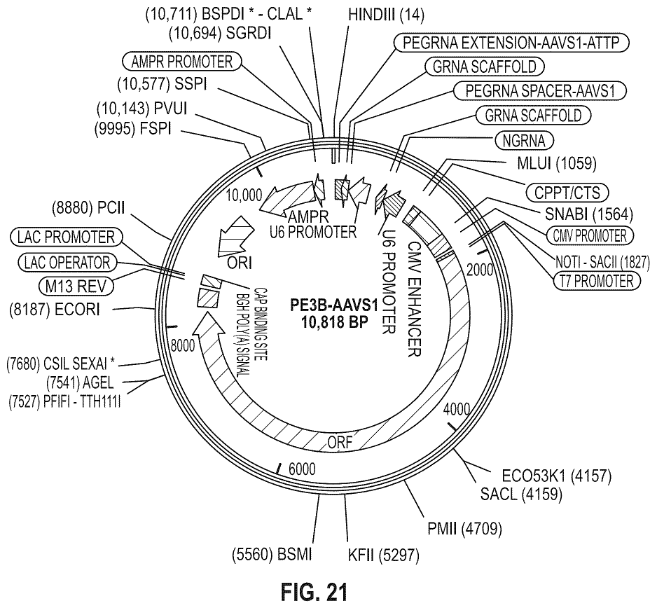

In one aspect, a first vector comprises the first set of first set of genomic editing components, a second vector comprises the second set of genomic editing components, and a third vector comprises the third set of genome editing components. A vector may be for example a plasmid vector. As described elsewhere herein, (e.g., FIG. 3 and FIG. 21), the sets of components are prepared as genetic constructs shown herein as plasmids, but it should be understood that the use of plasmids is not necessarily required. In one aspect, the first vector comprises or consists of the sequence of SEQ ID NO: 1, or SEQ ID NO: 25; the second vector comprises or consists of the sequence of SEQ ID NO: 11, or SEQ ID NO: 26; and the third vector comprises or consists of the sequence of SEQ ID NO: 19, or SEQ ID NO: 27.

In any of the methods and systems disclosed herein, the cell or cells may be selected from any cell for which modification is desired, and in various aspects are an immune cell, a neural cell or any stem cell, including a naïve stem cell, which may be a pluripotent or multipotent cell. In some aspects, the immune cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. In one aspect, the immune cells comprise T cells. As disclosed herein, T cells can be T cell of any subset, non-limiting examples can include T helper cells (TH cells), Cytotoxic T cells (TC cells, or CTLs), Memory T cells, Regulatory T cells (Treg cells), or Natural killer T (NKT) cells. In various aspects, the T cells can be enriched for one or more subsets based on cell surface expression. For example, in some cases, the T comprise are cytotoxic CD8+ T lymphocytes. In some embodiments, the T cells comprise γδ T cells, which possess a distinct T-cell receptor (TCR) having one γ chain and one δ chain instead of α and β chains.

The cell or cell populations may comprise or consist of resting immune cell(s), such resting T cell(s), resting NK cell(s), a cytotoxic T lymphocyte (CTL), and a regulatory T cell

In one aspect, the nucleic acid sequence of interest is an insertion. The insertion can be any nucleic acid sequence of interest which may vary considerably in length. By way of non-limiting example, the insertion is a nucleic acid sequence encoding a chimeric antigen receptor (CAR). A CAR can be inserted into the genome of resting cell(s) such as T-cells or NK cells by following the delivery steps of the method, and the cells then maintained cell for a time and under conditions sufficient for the cell to form a CAR-T cell or a CAR-NK cell.

A chimeric antigen receptor of the present disclosure may comprise one or more a hinge domain, an extracellular domain, signaling domain, intracellular domain, a spacer, costimulatory domain, a trans-membrane domain.

In one aspect, the hinge domain includes a hinge domain of a protein selected from CD28, 4-1BB (CD137), OX-40 (CD134), CD3ζ, T cell receptor α or β chain, CD45, CD4, CD5, CD8, CD8α, CD9, CD16, CD22, CD33, CD37, CD64, CD80, CD86, ICOS, CD154, functional derivatives and/or combinations thereof.

Typically, the extracellular domain, i.e., an antigen recognition domain or target element is linked to the intracellular domain, i.e., the co-stimulatory and signaling domain(s) of the chimeric antigen receptor by a transmembrane domain. A transmembrane domain traverses the cell membrane, anchors the CAR to the immune effector cell surface, and connects the extracellular domain to the intracellular domain, thus impacting expression of the CAR on the immune effector cell surface. The transmembrane domain includes a hydrophobic polypeptide that spans the cellular membrane. In particular, the transmembrane domain spans from one side of a cell membrane (extracellular) through to the other side of the cell membrane (intracellular or cytoplasmic). The transmembrane domain may be in the form of an alpha helix or a beta barrel, or combinations thereof. The transmembrane domain may include a polytopic protein, which has many transmembrane segments, each alpha-helical, beta sheets, or combinations thereof. In one aspect, the transmembrane domain that is naturally associated with one of the domains in the CAR is used. In another aspect, the transmembrane domain is selected or modified by amino acid substitution to avoid binding of such domains to the transmembrane domains of the same or different surface membrane proteins to minimize interactions with other members of the receptor complex. For example, the transmembrane domain is selected from a transmembrane domain of a T-cell receptor α or β chain, a CD3ζ chain, CD28, CD3s, CD45, CD4, CD5, CD7, CD8, CD9, CD16, CD22, CD33, CD37, CD64, CD80, CD86, CD68, OX-40 (CD134), 4-1BB (CD137), ICOS, CD41, CD154, functional derivatives and/or combinations thereof.

A chimeric antigen receptor of the present disclosure may further comprise one or more costimulatory domain and/or one or more spacers. A costimulatory domain is derived from costimulatory proteins that enhance cytokine production, proliferation, cytotoxicity, and/or persistence in vivo. In one aspect, the co-stimulatory domain is selected from OX-40 (CD134), CD27, CD28, CD30, CD40, PD-1, CD2, CD7, CD258, Natural killer Group 2 member C (NKG2C), Natural killer Group 2 member D (NKG2D), B7-H3, a ligand that binds to at least one of CD83, ICAM-1, LFA-1 (CD1 la/CD18), ICOS, and 4-1BB (CD137), CDS, ICAM-1, LFA-1 (CDla/CD18), CD40, CD27, active fragments thereof, functional derivatives thereof, and combinations thereof.

A chimeric antigen receptor of the present disclosure also comprises a signaling domain that provides an intracellular signal to the immune effector cell upon antigen binding to the antigen recognition domain. The signaling domain of a chimeric antigen receptor of the present disclosure is responsible for activation of at least one of the effector functions of the immune effector cell in which the chimeric receptor is expressed. The term “effector function” refers to a specialized function of a differentiated cell. An effector function of an immune cell, for example, may be cytolytic activity or helper activity including the secretion of cytokines. An effector function in a naive, memory, or memory-type immune cell may also include antigen-dependent proliferation. Thus, the term “intracellular domain” refers to the intracellular portion of a CAR that transduces the effector function signal upon binding of an antigen to the extracellular domain and directs the immune cell to perform a specialized function. Non-limiting examples of suitable signaling domains include the zeta chain of the immune-cell receptor or any of its homologs (e.g., eta, delta, gamma, or epsilon), MB-1 chain, 829, FcRIII, FcRI, and combinations of signaling molecules, such as CD37 and CD28, CD27, 4-1BB (CD137), DNAX-activating protein 10 (DAP10), OX-40 (CD134), and combinations thereof, as well as other similar molecules and fragments. Signaling domains of other activating proteins may be used, such as FcγRIII and FcϵRI. While usually the entire signaling domain will be employed, in many cases it will not be necessary to use the entire intracellular polypeptide. To that extent, a truncated portion of the signaling domain may be used as long as it still transduces the effector function signal. The term intracellular domain is also meant to include any truncated portion of the intracellular domain sufficient to transduce the effector function signal. Alternatively, or in addition, CAR-immune effector cells encompassed by the present disclosure may further comprise one or more suicide genes. As used herein, “suicide gene” refers to a nucleic acid sequence introduced to a CAR-immune effector cell by standard methods known in the art that, when activated, results in the death of the CAR-immune effector cell. Suicide genes may facilitate effective tracking and elimination of the CAR-immune effector cells in vivo if required. Facilitated killing by activating the suicide gene may occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In an exemplary embodiment, a suicide gene is a CD34/thymidine kinase chimeric suicide gene.

In another aspect, the present disclosure provides a non-viral system for modifying a cell, which comprises: (i) a first genetic construct comprising a gRNA nucleotide sequence capable of hybridizing to a target site, a pegRNA having an attP attachment site, a nickase or a nucleotide sequence encoding the nickase; (ii) a serine-integrase (PhiC31) or a second genetic construct comprising a nucleotide sequence encoding the serine-integrase (PhiC31); and (iii) a third genetic construct comprising a nucleotide sequence encoding the nucleic acid sequence of interest and an attB sequence. The first, second and third genetic constructs are non-viral, meaning that no viral genetic components are used. The genetic constructs may each respectively comprise a plasmid.

The CRISPR/CAS system, as disclosed herein can be optimized to allow insertion or deletion of genes at specific locations in the genome of the cell. Nucleotide sequence of a gRNA can be optimized to hybridize any desired target, for example an immunomodulating gene, a cancer antigen, or a gene known to modulate a disease or condition, such as TRAC, TRBC, HIF-1, PD-1 promoter regulatory region, or B2M. For In various aspects, a gRNA nucleotide sequence comprise a sequence capable of hybridizing a desired target site. By way of non-limiting example, gRNA nucleotide sequence comprises a sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with a sequence selected from the group consisting of SEQ ID NO: 28, SEQ ID NO: 29, SEQ ID NO: 30, and SEQ ID NO: 31. In one aspect, the gRNA nucleotide sequence comprise or consists essentially sequence of SEQ ID NO: 28, SEQ ID NO: 29, SEQ ID NO: 30, or SEQ ID NO: 31.

In various aspects, the delivery to the cell of the first, second and third sets of genome editing components uses microfluidic transfection devices and methods which are configured to enable multiple, e.g., two sequential transfection events. Cell(s) are combined in fluid medium with the first, second and third sets of genome editing components and passed through at least one microfluidic transfection device configured as described herein to enable at least two sequential transfection events.

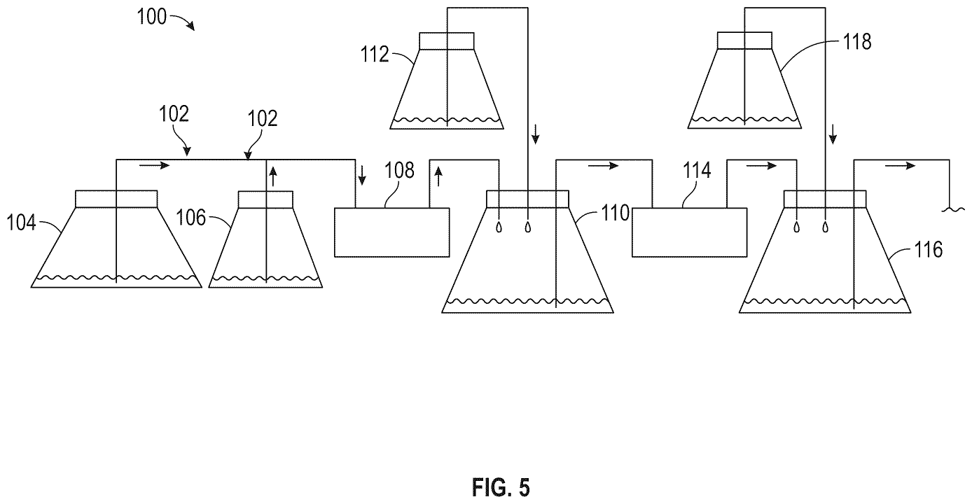

Thus, in one aspect and as shown in FIG. 5, delivery of the first set, second set and third set of components of the genome editing system is achieved using a sequential transfection device and methods. A system 100 may for example be a microfluidic system, which is configured for and operable to transfect a plurality of cells with at least one exogenous molecule. System 100 may include an initial fluid medium 102, a first reservoir 104, a first payload reservoir 106, a first microfluidic transfection device 108, a first holding chamber 110, a second payload reservoir 112, a second microfluidic transfection device 114, a second holding chamber 116, and a third payload reservoir 118. In one aspect, the first transfection device and the second transfection device are arranged in series so to deliver multiple exogenous molecules, i.e., multiple sets of genomic editing components to a cell or a plurality of cells.

As illustrated in FIG. 5, the first reservoir 104 may be in fluid communication with the initial fluid medium 102. The first reservoir 104 may contain a cell or a plurality of cells. The first reservoir 104 may be operable to provide the plurality of cells to the initial fluid medium 102. Cells may be moved from the first reservoir 104 to the initial fluid medium 102 by applying a flow rate using a flow rate supply to provide a cell velocity to the cells.

As illustrated in FIG. 5, the first payload reservoir 106 may be in fluid communication with the initial fluid medium 102. The first payload reservoir 106 may contain a first exogenous molecule to be transfected into a cell or a plurality of cells. The first payload reservoir 106 may inject the first exogenous molecule into the initial fluid medium 102 by using a flow rate supply. When the first exogenous molecule is delivered to the initial fluid medium 102, the first exogenous molecule and the cell or cells may form a first composition.

As illustrated in FIG. 5, the first microfluidic transfection device 108 may receive the first composition from the initial fluid medium 102. A flow rate may be supplied to pass the first composition through the first microfluidic transfection device 108, thereby producing a first transfected cell or first population of transfected cells. The first transfected cell or first population of transfected cells may then re-enter the initial fluid medium 102 and flow into the first holding chamber 110. The first microfluidic transfection device 108 may have optimized transfection parameters to transfect the cell or plurality of cells with the first exogenous molecule.

As illustrated in FIG. 5, the first holding chamber 110 may have an inlet in fluid communication with the initial fluid medium 102 and configured to receive the first population of transfected cells or first transfected cell, a payload inlet in fluid communication with a second payload reservoir 112 and configured to receive a second exogenous molecule into the first holding chamber 110, and an outlet in fluid communication with the initial fluid medium 102. In an example, the first population of transfected cells or first transfected cell may be optionally held in the holding chamber 110 for a sufficient period of time (e.g., a holding period) to allow the first population of transfected cells or first transfected cell to recover from the first transfection event.

As illustrated in FIG. 5, the second payload reservoir 112 may be in fluid communication with the payload inlet of the first holding chamber 110. The second payload reservoir 112 may provide a second exogenous molecule to the first population of transfected cells or first transfected cell in the first holding chamber 110. In an example, the second exogenous molecule and the first population of transfected cells or first transfected cell may be combined in the first holding chamber 110 to form a second composition.

As illustrated in FIG. 5, the second composition may be passed through a second microfluidic transfection device 114. The second microfluidic transfection device 114 may include an inlet configured to receive the second composition, a transfection component configured to transfect the second composition with the second exogenous molecule, thereby producing a second population of transfected cells or a second transfected cell, and an outlet configured to return the second population of transfected cells or a second transfected cell to the initial fluid medium. In an example, the second population of transfected cells or second transfected cell may comprise the first exogenous molecule and the second exogenous molecule.

The transfection component of the second microfluidic transfection device 114 may have transfection parameters optimized or tuned for the second transfection event (e.g., transfecting the cell or cells in the second composition with the second exogenous molecule). In an example, the transfection component of the second microfluidic transfection device 114 may have the same optimized transfection parameters as the transfection component of the first microfluidic transfection device 108. In another example, the transfection component of the second transfection device may have different transfection parameters than the transfection component of the first microfluidic transfection device 108.

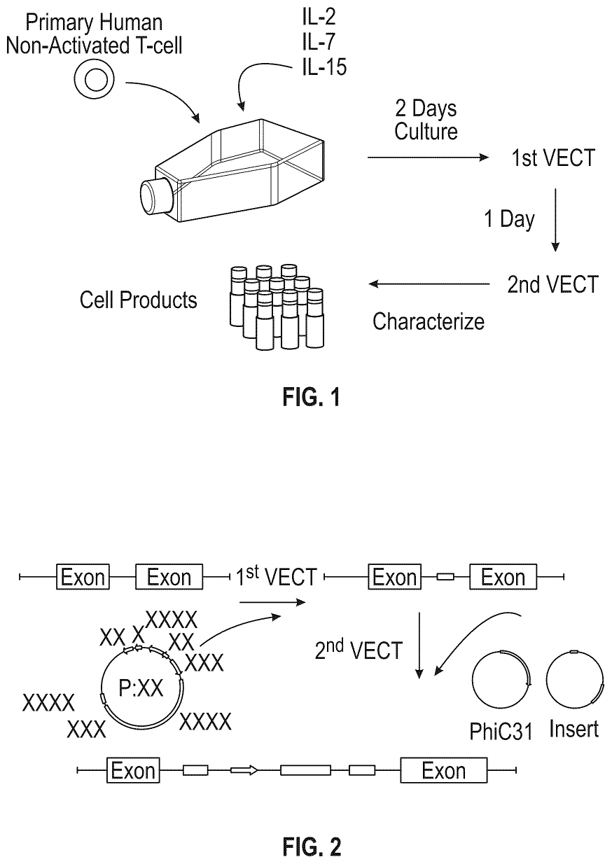

As illustrated in FIG. 5, after the second transfected cell or second population of transfected cells have been transfected by the second microfluidic transfection device 114, the cells are returned to the initial fluid medium 102 via the outlet in the second microfluidic transfection device 114. As illustrated in FIG. 1, the cells may flow through the initial fluid medium 102 to a second holding chamber 116 by providing a flow rate using a flow rate supply.

As illustrated in FIG. 5, the second holding chamber 116 may have an inlet in fluid communication with the initial fluid medium 102 configured to receive the second population of transfected cells or a second transfected cell, a payload inlet in fluid communication with a third payload reservoir 118 configured to receive a third exogenous molecule into the second holding chamber 116, and an outlet in fluid communication with the initial fluid medium 102. In an example, the second population of transfected cells or second transfected cell may be optionally held in the second holding chamber 116 for a holding period.

The second holding chamber 116 may have an outlet configured to remove the second transfected cell or second population of transfected cells from the system if only two transfection events are desired. In another example, the second population of transfected cells or second transfected cell may undergo a third transfection event.

As illustrated in FIG. 5, the third payload reservoir 118 may be in fluid communication with the payload inlet of the second holding chamber 116. The third payload reservoir 118 may provide a third exogenous molecule to the second population of transfected cells in the second holding chamber. In an example, the third exogenous molecule and the second population of transfected cells or second transfected cell may be combined in the second holding chamber to form a third composition.

A flow rate supply may move the third composition from the second holding chamber 116 into the initial fluid medium 102 by providing a cell velocity to the third composition. The third composition may move through the initial fluid medium 102 to a third microfluidic transfection device (not shown). The third microfluidic transfection device may be operable to transfect the cells in the third composition with the third exogenous molecule (e.g., third transfection event). The third microfluidic transfection device may have transfection parameters tuned or optimized for the third transfection event.

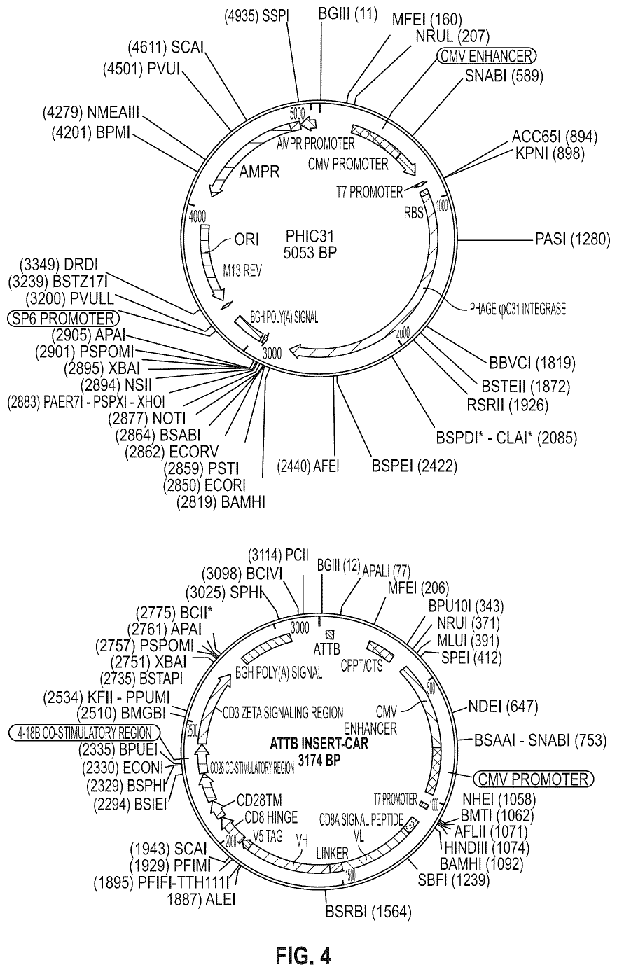

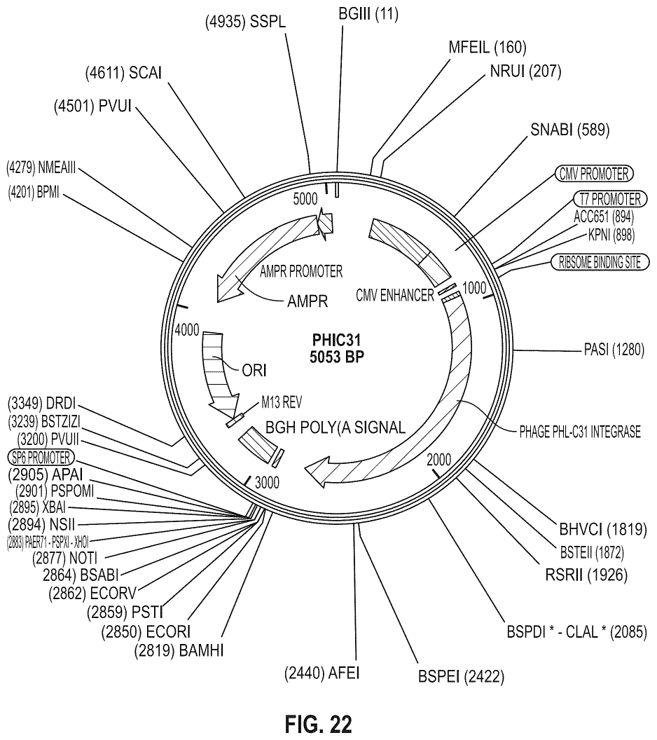

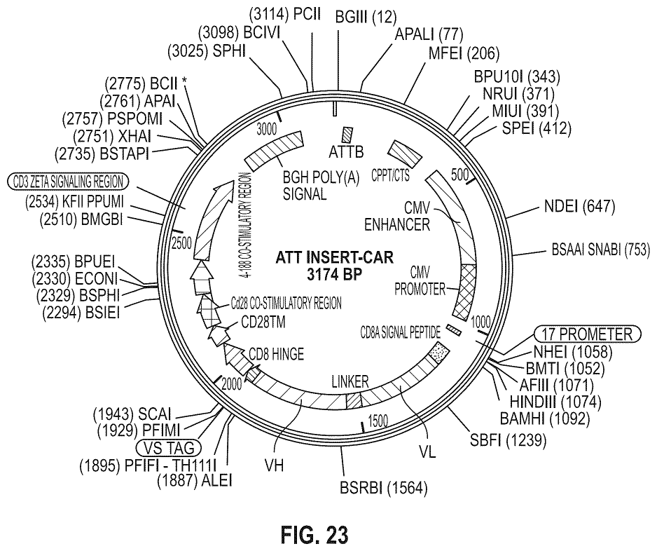

Subsequently, the second VECT event is performed using the plasmids shown in FIG. 4 for a co-transfection that includes an overexpression plasmid for PhiC31 (serine integrase) (FIG. 22) and a plasmid serving as an insertion cassette containing CAR construct and an attB docking sequencing (FIG. 23). The overall workflow is illustrated in FIG. 1 to show the expected timeline for primary T-cell culture and the two-steps process which indicates the modified locus as a result of this procedure (FIG. 2). As shown in FIG. 2, “VECT events” are transfection events, which in an exemplary aspect are achieved using a sequential microfluidic transfection method and system as disclosed herein. As shown in FIGS. 3 and 4, the genetic constructs are presented as plasmid vectors, but it should be understood that the use of plasmids is not necessarily required. Notably, use of the disclosed sequential microfluidic transfection methods enables delivery of the first, second and third sets of components of the genomic editing system to the cell in a period of no more than 144 hours, 120 hours, 96 hours, 72 hours, 60 hours, 48 hours, 36 hours, 24 hours, 16 hours, 8 hours, 6 hours and 1 hour.

### (ii) Cells and Cell Populations

In various other aspects, the present disclosure also provides cells which are made by any of the disclosed methods, using any of the disclosed methods or systems, or which comprise any of the disclosed systems. Cells as described herein may be contained with any microfluidic device as described herein.

The present disclosure encompasses a cell or a population of cells in a fluid medium in combination with any one or more of the genetic constructs disclosed herein, such as any one or all of: (i) a first genetic construct comprising the guide RNA nucleotide sequence pegRNA having an attP site, and a nickase or a nucleotide sequence encoding the nickase; (ii) the serine-integrase (PhiC31) or a second genetic construct comprising a nucleotide sequence encoding the serine-integrase (PhiC31); and (iii) a third genetic construct comprising a nucleotide sequence encoding the nucleic acid sequence of interest and an attB sequence.

In one aspect it should be noted that any of the cells or a cell population need not be purified or isolated from other components that that be contained in a donated cell population from which the cells are obtained. In one aspect, cells are prepared for the multiple transfection events as disclosed herein by maintaining the cells for a period under cell culture conditions including a culture medium comprising at least one of interleukin (IL)-2, IL-7 and IL-15, or any combination thereof. The period may be 1-5 days, and in one aspect is a period of about 2 days, or at least about 2 days.

As noted elsewhere herein, any of the cells or may be resting or inactivated cells, such as resting T-cells or NK cells. Cells that may be made by any of the disclosed methods, using any of the disclosed methods or systems, or which comprise any of the disclosed systems, or cells contained within any microfluidic device as described herein may comprise CAR-T or CAR-NK cell or cells.

The present disclosure contemplates that using a sequential microfluidics approach as described herein, cells are completely transfected within a period of about 5-6 days.

In another aspect, the method may be conducted in a chemical transfection system. The chemical transfection system may use a transfection reagent to deliver a genetic compound (e.g., exogenous molecule) to a cell or plurality of cells. Liposomal-based transfection reagents may be used to enable the formation of positively charged lipid aggregates that may merge smoothly with the phospholipid bilayer of the host cell to allow the entry of a genetic compound (e.g., exogenous molecule) into a cell with minimal resistance. In other examples, non-liposomal transfection reagents may additionally or alternatively be used. In some examples, the non-liposomal transfection reagents may comprise calcium phosphate, dendrimers, polymers, nanoparticles, and/or non-liposomal lipids.

In a further aspect, an electroporation transfection system may be used to conduct the method. In an electroporation transfection system, electrical pulses are used to create temporary pores in cell membranes. Genetic compounds (e.g., exogenous molecules) may be inserted through the pores in the cell membrane to transfect the cells.

Regardless of the transfection method used, successful transfection and resulting modification using the systems described herein can be determined using any of a number of known techniques for identifying the presence of an exogenous nucleic acid or exogenous polypeptide sequence in the cell. For example, insertion of an exogenous nucleic acid sequence can be detected by performing genomic nucleic acid sequencing or PCR analysis on the cell contents. Alternatively, a reporter gene can be integrated, and cells can be subjected to flow cytometry or imaging acquisition to detect the reporter gene expression which is indicative of exogenous nucleic acid expression. Alternatively, flow cytometry analysis or imaging acquisition can be used to detect an antibody or fluorophore tag to detect the presence of an exogenous polypeptide sequence.

### (iii) Microfluidic Transfection Systems and Methods

Also disclosed herein are systems and methods in various configurations for sequential microfluidic processing of cells. The system is configured for sequential processing of a cell or cells through a microfluidic transfection device to transfect the cell or cells with at least one exogenous molecule, and in the context of the present disclosure can be usefully configured to efficiently introduce multiple different exogenous molecules into a cell, such as the multiple plasmid modification system described herein for modifying resting cells such as resting T-cells. It will be appreciated that other transfection systems may be used to perform the cell modifying methods and systems disclosed herein.

The systems and methods provided herein allow all materials to be delivered intracellularly using back-to-back transfection events with minimal impact to cell viability over several events and retention of high delivery efficiency to the cells. Typically, performing this process back-to-back with existing technologies results in low cell viability and/or low delivery efficiency. Using the systems and methods described herein cells can be transfected back-to-back and retain high enough cell numbers and high enough cell health to allow for rapidly following one transfection event with a second transfection event. Using the systems and methods herein the resting time between transfection events can be less than one hour, and in some cases, there may be no resting time at all. The sequential nature of the systems and methods described herein produce a high cell viability and a high rate of transfection. The systems and methods herein are designed for sequential microfluidic processing and therefore may not require an expansion. The systems may be self-contained and the methods using the systems may be conducted in the self-contained environment of the systems.

It will be appreciated that other transfection systems may be used. The following transfection systems are provided as exemplary transfection systems for use in the disclosed methods of modifying a cell.

FIG. 5 illustrates a system for high throughput introduction of a plurality of exogenous molecules into a cell. The system may be configured to allow for multiple exogenous molecules to be transfecting into a cell sequentially. The sequential nature of the system may produce a transfected cell comprising multiple exogenous molecules in a short period of time. Two or more transfection events may be successfully completed in under an hour.

As illustrated in FIG. 5, the system 100 may include an initial fluid medium 102, a first reservoir 104, a first payload reservoir 106, a first microfluidic transfection device 108, a first holding chamber 110, a second payload reservoir 112, a second microfluidic transfection device 114, a second holding chamber 116, and a third payload reservoir 118. The system 100 may be operable to transfect a plurality of cells with at least one exogenous molecule. The first transfection device and the second transfection device may be arranged in series. The system 100, as illustrated in FIG. 1, is configured to deliver a first exogenous molecule and a second exogenous molecule to a cell or plurality of cells.

As illustrated in FIG. 5, the first reservoir 104 may be in fluid communication with the initial fluid medium 102. The first reservoir 104 may contain a cell or a plurality of cells. In an example, the cells may be homogenous. The first reservoir 104 may be operable to provide the plurality of cells to the initial fluid medium 102. Cells may be moved from the first reservoir 104 to the initial fluid medium 102 by applying a flow rate using a flow rate supply to provide a cell velocity to the cells.

As illustrated in FIG. 5, the first payload reservoir 106 may be in fluid communication with the initial fluid medium 102. The first payload reservoir 106 may contain a first exogenous molecule to be transfected into a cell. The first payload reservoir 106 may inject the exogenous molecule into the initial fluid medium 102 by using a flow rate supply. When the first exogenous molecule is delivered to the initial fluid medium 102, the first exogenous molecule and the cells may form a first composition.

The first exogenous molecule may comprise gene editing materials, nanoparticles, protein, antigens, amino-acids, viruses or viral components, DNA and related material, RNA and related material, lipids and related materials, small molecules, and salts. In an example, the first exogenous molecule may comprise a nucleotide sequence encoding an amino acid sequence of interest. It will be appreciated that the types of exogenous molecules may be used in all of the systems and methods described herein. In some examples, the first exogenous molecule may be sgRNA and the second exogenous molecule may be CRISPR associated protein 9 (Cas9). In some examples, the first or second exogenous molecule may be a lyophilized gRNA. In other examples, the first exogenous molecule may be Cas9. In some examples, the first exogenous molecule may include two or more exogenous molecules comprising a vector. The types of exogenous molecules described in this paragraph may be used as a first, second, third, fourth, fifth, or Nth exogenous molecule to be transfected into a cell or a plurality of cells using any of the systems or methods described herein.

In some aspects, the first exogenous molecule may be used for a knock-out gene editing therapy. In an example, the second exogenous molecule may be used for a knock-in gene therapy. In other examples, either exogenous molecule may be used for a knock-in or a knock-out gene editing therapy. In some examples, the first exogenous molecule and the second exogenous molecule may be used in combination to perform a gene editing therapy. In some examples, the first exogenous molecule may be a plasmid configured to insert an attP sequence in a desired locus of a cell. The second exogenous molecule may be a plasmid configured to perform an attB docking sequence. For example, a plasmid may comprise units to express the nCas9-M-MLV fusion protein along with expression modules of pegRNA and gRNA to perform a CRISPR prime editing for inserting an attP site in the desired locus. A second plasmid may comprise PhiC31 (serine integrase). A third plasmid may be an insertion cassette containing CAR construct and an attB docking sequence.

In another aspect, the first exogenous molecule and the second exogenous molecule may be configured to produce a gene edit on a cell. In some examples, the first exogenous molecule may prepare the cell or cells for insertion of the second exogenous molecule.

As illustrated in FIG. 5, the first composition may be passed through a first microfluidic transfection device 108. The first microfluidic transfection device 108 may include an inlet configured to receive the first composition, a transfection component configured to transfect the cell or cells with the first exogenous molecule, thereby producing a first population of transfected cells or a first transfected cell, and an outlet configured to return the first population of transfected cells to the initial fluid medium. In an example, the first population of transfected cells or first transfected cell may comprise the first exogenous molecule.

The cell or cells may have a successful transfection percentage of about 20%, about 25%, about 30%, about 35%, or about 40% after being transfected using the first transfection device. In an example, the cell or cells may have a successful transfection percentage of about 20% to about 40%. About 50%, about 55%, about 60%, about 65%, about 70%, about 75%, about 80%, about 85%, or about 90% of the cells may remain viable after being transfected using the first transfection device. In an example, about 50% to about 90% of the cells may remain viable after being transfected using the first transfection device.

The transfection components of the microfluidic transfection devices disclosed herein may have various transfection parameters optimized for transfecting the cell or cells (e.g., to produce a high viability rate and a high successful transfection rate). In an example, the transfection parameters for optimized transfection of the cell or cells with an exogenous molecule may be device gap, supply pressure, supply flow rate, cell velocity, ridge number, channel width, channel height, channel length, gap width, gap size (e.g., height), gap length, ridge spacing, ridge angle, temperature, processing buffer constituents, cell type, cell source, increasing or decreasing the driving fluid pressure, and number of parallelized channels. Optimization, types, and functions of microfluidic transfection devices are described in U.S. Publication No. 2021/0292700A1, which is incorporated herein by reference in its entirety. It will be appreciated that the optimization or tuning of the microfluidic transfection device described in this paragraph may be applied to any of the microfluidic transfection devices in this system or in any other system or method described herein. Each microfluidic transfection device described in this system, or any other system or method described herein, may have optimized or tuned transfection parameters for a transfection event depending on the size, stiffness, FACS characteristics, other physical characteristics (e.g., color, shape, etc.), or necessary or desired compression of the cell or cells to be transfected.

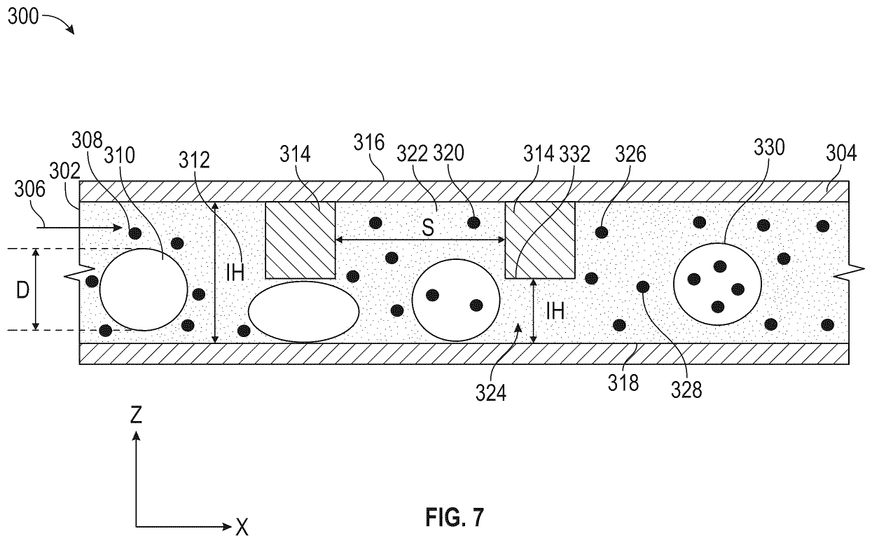

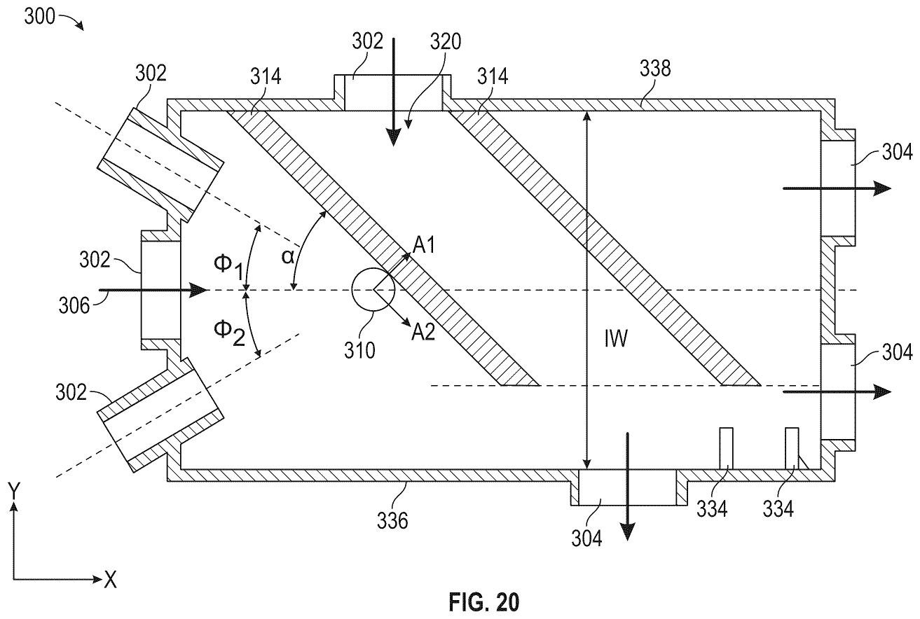

FIGS. 7 and 20 illustrate an example microfluidic transfection device 300 (e.g., first microfluidic transfection device 108 and/or second microfluidic transfection device 114). The microfluidic transfection device may have one or more inlets 302 and one or more outlets 304. The arrow 306 illustrates the direction of the flow of cells 310, exogenous molecules 308, and/or compositions through the microfluidic transfection device 300. The microfluidic transfection device 300 may include a liquid media 312. The microfluidic transfection device 300 may include one or more ridges 314. The microfluidic transfection device 300 may have a first wall 316 (e.g., top wall) having a first interior surface 326 and a second wall 318 (e.g., bottom wall) having a second interior surface 328. The microfluidic transfection device 300 may have a third wall 336 (e.g., first side wall) and a fourth wall 338 (e.g., second side wall). The microfluidic transfection device 300 may have a recovery space 320. The ridges 314 may have a ridge spacing 322. The ridges 314 may have a ridge surface 332. The microfluidic transfection device 300 may have a gap 324. The microfluidic transfection device 300 may have cell counters 334. After passing through the components of the microfluidic transfection device 300, a transfected cell 330 may be produced.

In an aspect, the first wall 316, second wall 318, third wall 336, and fourth wall 338 may enclose the microfluidic transfection device 300 to have an interior height (IH) and an interior width (IW), as illustrated, for example, in FIGS. 7 and 20.

The inlets 302 may be located at different locations and at different angles, as illustrated, for example, in FIG. 20. In some examples, the angle (φ1 and/or φ2) may be between 20 degrees and 80 degrees. In some examples, the angle (φ1 and/or φ2) may be about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, about 45 degrees to about 50 degrees, about 50 degrees to about 55 degrees, about 55 degrees to about 60 degrees, about 60 degrees to about 65 degrees, about 65 degrees to about 70 degrees, about 70 degrees to about 75 degrees, about 75 degrees to about 80 degrees, about 80 degrees to about 85 degrees, or about 85 degrees to about 90 degrees. In some examples, one or more inlets 302 may be located at a point after one of the ridges 314, as illustrated, for example, in FIG. 20.

The gap 324 of the transfection device 300 (e.g., 108, 114) may be the distance between the surface 332 of the ridges 314 and the second interior surface 328. The gap 324 may be smaller than the height of the cell or cells to be transfected. The transfection component of the microfluidic transfection device 300 may have a gap 324 of about 5.4 μm to about 5.6 μm. In a further example, the transfection component may have a gap 324 of about 0.5 μm to about 1 μm, about 1 μm to about 1.5 μm, about 1.5 μm to about 2 μm, about 2 μm to about 2.5 μm, about 2.5 μm to about 3 μm, about 3 μm to about 3.5 μm, about 4 μm to about 4.5 μm, about 4.5 μm to about 5 μm, about 5 μm to about 5.5 μm, about 5.5 μm to about 6 μm, about 6 μm to about 6.5 μm, about 6.5 μm to about 7 μm, about 7 μm to about 7.5 μm, about 7.5 μm to about 8 μm, about 8 μm to about 8.5 μm, about 8.5 μm to about 9 μm, about 9 μm to about 9.5 μm, or about 9.5 μm to about 10 μm. In another example, the gap 324 may be about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, or about 90 μm to about 100 μm. In an example, the gap 324 may be optimized based on the type and size of the cell, the compression needed or desired, and other characteristics of microfluidic transfection. By optimizing the gap 324 of the transfection component of the microfluidic transfection device 300, the viability percentage of cells after transfection and the successful transfection percentage of cells may be optimized. In an example, the gap size may be optimized by decreasing the gap size to a value lower than the size of the cell or cells to be transfected (e.g., smaller cells have smaller gap sizes and larger cells have larger gap sizes). In an example, the optimized gap 324 for a T-cell may be about 5.3 μm.

As illustrated in FIGS. 7 and 20, cells may be transfected by flowing through the gap 324 along flow path A1. Abnormal cells (e.g., cells with low compressibility) may flow along flow path A2 and out of the microfluidic transfection device 300 without being transfected. By flowing abnormal cells out of the microfluidic transfection device 300 without allowing them to pass through the gap, clogging or other negative effects may be prevented.

The transfection component of the microfluidic transfection device 300 may have a gap width and gap length (e.g., length of the ridge surface 332) optimized for transfection of the cell or cells with the first exogenous molecule depending on the size, stiffness, FACS characteristics, other physical cell properties of the cell or cells or the compression necessary or desired for the transfection event. In an example, the optimal gap width and gap length may depend on the type of cell and the type of exogenous molecule. In an example, the gap width may be about 5 μm and the gap length may be about 5 μm. In some examples, the gap width may be about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, about 90 μm to about 100 μm, about 100 μm to about 200 μm, about 200 μm to about 300 μm, about 300 μm to about 400 μm, or about 400 μm to about 500 μm. In some examples, the gap length (e.g., ridge surface 332 length) may be about 1 μm to about 2 μm, about 2 μm to about 3 μm, about 3 μm to about 4 μm, about 4 μm to about 5 μm, about 5 μm to about 6 μm, about 6 μm to about 7 μm, about 7 μm to about 8 μm, about 8 μm to about 9 μm, about 9 μm to about 10 μm, about 10 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, or about 700 μm to about 800 μm. In an example, for a T-cell the optimal gap width may be 12 μm and the optimal gap length may be 12 μm.

The transfection component of the microfluidic transfection device 300 may have a channel cross-sectional dimension and channel length optimized for transfection of the cell or cells with the first exogenous molecule depending on the size, stiffness, FACS characteristics, or other physical properties of the cell or cells. In an example, the optimal channel cross-sectional dimension and channel length may depend on the type of cell. In an example, the channel cross-section dimension may be about 1 μm to about 10 μm, about 10 μm to about 20 μm, about 20 μm to about 30 μm, about 30 μm to about 40 μm, about 40 μm to about 50 μm, about 60 μm to about 70 μm, about 70 μm to about 80 μm, about 80 μm to about 90 μm, about 90 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700, about 700 μm to about 800 μm, about 800 μm to about 900 μm, about 900 μm to about 1000 μm, about 1000 to about 1250 μm, about 1250 μm to about 1500 μm, about 1500 μm to about 1750 μm, or about 1750 μm to about 2000 μm. The channel length may be about 1 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 2000 μm, about 2000 μm to about 3000 μm, about 3000 μm to about 4000 μm, about 4000 μm to about 5000 μm, or more.

The transfection component of the microfluidic transfection device 300 may have an optimal number of ridges to transfect the cell or cells with an exogenous molecule depending on the size, stiffness, FACS characteristics, or other physical properties of the cell or cells. In an example, the first transfection component may have about 1, 2, 3, 4, 5, 6, 7, 8, 9, 10, 11, 12, 13, 14, 15, 16, or more ridges. For example, T-cells may exhibit the same or better results for 1 ridge when compared to 5. There are other processing metrics aside from cell metrics (i.e., viability and transfection) to using 1 ridge, including reduced running pressure and pressure requirements overall, higher throughput, etc. However, for other cell types different ridge numbers may perform better. Very early testing of peripheral blood mononuclear cells shows generally better results with 5 ridges than 1 ridge.

In an aspect, the ridges 314 may be rectangular and extend into the microfluidic transfection device 300 at a 90 degree angle from the first interior surface 326. As illustrated in FIG. 20, the ridges 314 may extend into the microfluidic transfection device 300 at an angle α. In some examples, the angle α may be about a 10 degree angle, about a 20 degree angle, about a 30 degree angle, about a 40 degree angle, about a 50 degree angle, about a 60 degree angle, about a 70 degree angle, about an 80 degree angle, or about a 90 degree angle from the first interior surface 326. In other examples, the ridges 314 may be rounded, pointed, or have any other shape.

In an aspect, the ridges 314 may have a surface roughness at the ridge surface 332. In some examples, the ridges 314 may have a surface roughness of about 1 nm to about 10 nm, about 10 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 200 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 900 nm to about 1000 nm, about 1000 nm to about 1500 nm, or about 1500 nm to about 2000 nm.

The transfection component of the microfluidic transfection device 300 may have optimal ridge spacing 322 for transfection of the cell or cells with an exogenous molecule depending on the size, stiffness, FACS characteristics, or other physical properties of the cell or cells. In an example, the optimal ridge spacing 322 may depend on the type of cell and the type of first exogenous molecule. In an example, the optimal ridge spacing may be about 100 μm. In some examples, the ridge spacing 322 may be about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 5000 μm, or about 5000 μm to about 50000 μm.

The system may include processing buffer constituents. In an example, the processing buffer constituents may be selected based on cell type (e.g., size, stiffness, FACS characteristics, and/or other physical characteristics of the cell or cells). The processing buffer constituents may be selected from buffer constituents configured for microfluidic transfection devices. For example, a basal cell culture medium may be the processing buffer. In other examples, buffer constituents may be chosen based on whether the cells are primary cells (i.e., from a patient) or from cell lines (i.e., immortal test cell types).

In another aspect, the microfluidic transfection device 300 may have multiple parallelized channels for transfecting the cell or cells with an exogenous molecule. In some examples, the microfluidic transfection device 300 may have about 2, 3, 4, 5, 6, 7, 8, 9, 10, 11, 12, 13, 14, 15, 16, 17, 18, 19, or 20 parallelized channels. In other examples, the microfluidic transfection device 300 may have about 1 to about 10 parallelized channels, about 10 to about 100 parallelized channels, about 100 to about 500 parallelized channels, about 500 to about 1000 parallelized channels, about 1000 to about 5000 parallelized channels, about 5000 to about 15000 parallelized channels, or more parallelized channels.

Referring back to FIG. 5, the first microfluidic transfection device 108 may have optimized transfection parameters for transfecting the cell or cells with the first exogenous molecule.

Referring to FIG. 5, after the cell or cells have been transfected by the first microfluidic transfection device 108 (e.g., first transfection event), the cell or cells are returned to the initial fluid medium 102 via the outlet in the first microfluidic transfection device 108. As illustrated in FIG. 1, the cell or cells may flow through the initial fluid medium 102 to a first holding chamber 110.

As illustrated in FIG. 5, the first holding chamber 110 may have an inlet in fluid communication with the initial fluid medium 102 and configured to receive the first population of transfected cells or first transfected cell, a payload inlet in fluid communication with a second payload reservoir 112 and configured to receive a second exogenous molecule into the first holding chamber 110, and an outlet in fluid communication with the initial fluid medium 102. In an example, the first population of transfected cells or first transfected cell may be optionally held in the holding chamber 110 for a sufficient period of time (e.g., a holding period) to allow the first population of transfected cells or first transfected cell to recover from the first transfection event. In some examples, the holding period may be about 0.1 seconds to about 60 minutes. In a further example, the holding period may be about 0.1 seconds to about 1 second, about 1 second to about 10 seconds, about 10 seconds to about 20 seconds, about 20 seconds to about 30 seconds, about 30 seconds to about 40 seconds, about 40 seconds to about 50 seconds, about 50 seconds to about 60 seconds, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes. In some examples, the holding period can allow the cells sufficient time and conditions to express one or more nucleotide sequences. In another example, the cells may be immediately injected with a second payload when they reach the holding chamber and have no holding period (e.g., no intervening culture step). It will be appreciated that the holding period as described in this paragraph may be applied to any system or method described herein.

As illustrated in FIG. 5, the second payload reservoir 112 may be in fluid communication with the payload inlet of the first holding chamber 110. The second payload reservoir 112 may provide a second exogenous molecule to the first population of transfected cells or first transfected cell in the first holding chamber 110. In an example, the second exogenous molecule and the first population of transfected cells or first transfected cell may be combined in the first holding chamber 110 to form a second composition. In one example, the second exogenous molecule may be a different exogenous molecule from the first exogenous molecule. In another example, the first exogenous molecule and the second exogenous molecule may be the same exogenous molecule.

A flow rate supply may move the second composition from the first holding chamber 110 into the initial fluid medium 102 by providing a cell velocity to the second composition. In an example, the second composition may move through the initial fluid medium 102 to the second microfluidic transfection device 114.

As illustrated in FIG. 5, the second composition may be passed through a second microfluidic transfection device 114. The second microfluidic transfection device 114 may include an inlet configured to receive the second composition, a transfection component configured to transfect the second composition with the second exogenous molecule, thereby producing a second population of transfected cells or a second transfected cell, and an outlet configured to return the second population of transfected cells or a second transfected cell to the initial fluid medium. In an example, the second population of transfected cells or second transfected cell may comprise the first exogenous molecule and the second exogenous molecule.

The cell or cells may have a successful transfection percentage of about 20%, about 25%, about 30%, about 35%, or about 40% after being transfected using the second transfection device. In an example, the cell or cells may have a successful transfection percentage of about 20% to about 40% using the second transfection device. About 50%, about 55%, about 60%, about 65%, about 70%, about 75%, about 80%, about 85%, or about 90% of the cells may remain viable after being transfected using the second transfection device. In an example, about 50% to about 90% of the cells may remain viable after being transfected using the second transfection device.

The transfection component of the second microfluidic transfection device 114 may have transfection parameters optimized or tuned for the second transfection event (e.g., transfecting the cell or cells in the second composition with the second exogenous molecule). In an example, the transfection component of the second microfluidic transfection device 114 may have the same optimized transfection parameters as the transfection component of the first microfluidic transfection device 108. In another example, the transfection component of the second transfection device may have different transfection parameters than the transfection component of the first microfluidic transfection device 108.

As illustrated in FIG. 5, after the cell or cells have been transfected by the second microfluidic transfection device 114, the cells are returned to the initial fluid medium 102 via the outlet in the second microfluidic transfection device 114. As illustrated in FIG. 1, the cells may flow through the initial fluid medium 102 to a second holding chamber 116 by providing a flow rate using a flow rate supply.

As illustrated in FIG. 5, the second holding chamber 116 may have an inlet in fluid communication with the initial fluid medium 102 configured to receive the second population of transfected cells or a second transfected cell, a payload inlet in fluid communication with a third payload reservoir 118 configured to receive a third exogenous molecule into the second holding chamber 116, and an outlet in fluid communication with the initial fluid medium 102. In an example, the second population of transfected cells or second transfected cell may be optionally held in the second holding chamber for a holding period. In another example, the cells may be immediately injected with a third payload when they reach the second holding chamber 116 and have no holding period (e.g., no intervening culture step).

The second holding chamber 116 may have an outlet configured to remove the cell or cells from the system if only two transfection events are desired. In another example, the second population of cells or second transfected cell may undergo a third transfection event.

As illustrated in FIG. 5, the third payload reservoir 118 may be in fluid communication with the payload inlet of the second holding chamber 116. The third payload reservoir 118 may provide a third exogenous molecule to the second population of transfected cells in the second holding chamber. In an example, the third exogenous molecule and the second population of transfected cells or second transfected cell may be combined in the second holding chamber to form a third composition. In one example, the third exogenous molecule may be a different exogenous molecule from the first exogenous molecule and/or second exogenous molecule. In another example, the first exogenous molecule, second exogenous molecule, and third exogenous molecule may be the same exogenous molecule. In a further example, the first exogenous molecule may be the same as the second exogenous molecule but different than the third exogenous molecule. In another example, the second exogenous molecule and the third exogenous molecule may be the same exogenous molecule, and the first exogenous molecule may be different from the second exogenous molecule and the third exogenous molecule.

A flow rate supply may move the third composition from the second holding chamber 116 into the initial fluid medium 102 by providing a cell velocity to the cell or cells. The third composition may move through the initial fluid medium to a third microfluidic transfection device (not shown). The third microfluidic transfection device may be operable to transfect the cells in the third composition with the third exogenous molecule (e.g., third transfection event). The third microfluidic transfection device may have transfection parameters tuned or optimized for the third transfection event.

The system may include a flow rate supply. In an example, the flow rate supply may be any device or instrument that provides a velocity to the cells in the system. The flow rate supply may provide an optimized cell velocity for a transfection event. In some examples, the flow rate supply may be configured to supply a cell velocity of about 1 mm/s to about 2 mm/s, about 2 mm/s to about 3 mm/s, about 3 mm/s to about 4 mm/s, about 4 mm/s to about 5 mm/s, about 5 mm/s to about 6 mm/s, about 6 mm/s to about 7 mm/s, about 7 mm/s to about 8 mm/s, about 8 mm/s to about 9 mm/s, about 9 mm/s to about 10 mm/s, about 10 mm/s to about 20 mm/s, about 20 mm/s to about 30 mm/s, about 30 mm/s to about 40 mm/s, about 40 mm/s to about 50 mm/s, about 50 mm/s to about 60 mm/s, about 60 mm/s to about 70 mm/s, about 70 mm/s to about 80 mm/s, about 80 mm/s to about 90 mm/s, about 90 mm/s to about 100 mm/s, about 100 mm/s to about 200 mm/s, about 200 mm/s to about 300 mm/s, about 300 mm/s to about 400 mm/s, or about 400 mm/s to about 500 mm/s. The velocity may be optimized depending on the characteristics of the cell or cells to be transfected.

In one example, the flow rate supply may be a pressure supply to apply a pressure of about 5 psi to about 100 psi to the system to move the cells between components. In an example, the pressure supply may supply a pressure of about 5 psi, about 10 psi, about 15 psi, about 20 psi, about 25 psi, about 30 psi, about 35 psi, about 40 psi, about 45 psi, about 50 psi, about 55 psi, about 60 psi, about 65 psi, about 70 psi, about 75 psi, about 80 psi, about 85 psi, about 90 psi, about 95 psi, or about 100 psi. In another example, the pressure supply may supply a pressure of about 5 psi to about 10 psi, about 10 psi to about 15 psi, about 15 psi to about 20 psi, about 20 psi to about 25 psi, about 25 psi to about 30 psi, about 30 psi to about 35 psi, about 35 psi to about 40 psi, about 40 psi to about 45 psi, about 45 psi to about 50 psi, about 50 psi to about 55 psi, about 55 psi to about 60 psi, about 60 psi to about 65 psi, about 65 psi to about 70 psi, about 70 psi to about 75 psi, about 75 psi to about 80 psi, about 80 psi to about 85 psi, about 85 psi to about 90 psi, about 90 psi to about 95 psi, about 95 psi to about 100 psi. In some examples, the flow rate supply may supply a different flow rate for different movements of the cell or cells depending on a desired velocity. It will be appreciated that the flow rate supply described in this paragraph may be used in any of the other systems and methods described herein.

The pressure supply may comprise a regulator having multiple control systems. In an example, the regulator may provide pressure at locations where the cells need to move. The regulator may supply pressure at a first location (e.g., the first cell reservoir) to move the cell or cells from the first reservoir 104 to the initial fluid medium 102 and through the first microfluidic transfection device 108 to the first holding chamber 110. The regulator may supply a pressure at a second location (e.g., the first holding chamber 110) to move the second composition of cells from the first holding chamber 110 to the initial fluid medium 102 and through the second microfluidic transfection device 114 to the second holding chamber. The regulator may supply a pressure at a third location (e.g., the second holding chamber) to move the third composition of cells from the second holding chamber 116 to the initial fluid medium 102 and through the third microfluidic transfection device.

In another aspect, the system 100 may include multiple pressure supplies. Each pressure supply may have a regulator. In an example, a first pressure supply may have a regulator that may supply a pressure at a first location (e.g., the first cell reservoir) to move the cell or cells from the first reservoir 104 to the initial fluid medium 102 and through the first microfluidic transfection device 108 to the first holding chamber 110. A second pressure supply may have a regulator that may supply a pressure at a second location (e.g., the first holding chamber) to move the second composition of cells from the first holding chamber 110 to the initial fluid medium 102 and through the second microfluidic transfection device 114 to the second holding chamber 116. A third pressure supply may supply a pressure at a third location (e.g., the second holding chamber 116) to move the third composition of cells from the second holding chamber 116 to the initial fluid medium 102 and through the third microfluidic transfection device.

The system may include N holding chambers, transfection devices, and payload reservoirs configured to transfect N exogenous molecules into the cell or cells. The N holding chambers, transfection devices, and payload reservoirs may have the same structures, characteristics and properties as the first and second holding chambers, transfection devices, and payload reservoirs. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Further provided herein is a method for high throughput introduction of a plurality of exogenous molecules into a cell. The method may produce transfected cells comprising one or more exogenous molecules at a rapid pace. A first transfection event and a second transfection event may be completed in under an hour. The transfected cell or cells may be transfected with two or more exogenous molecules sequentially and produce high transfection rate (e.g., percentage) and high viability percentage compared to methods known in the art. The method may be completed in a fraction of the time of methods known in the art.

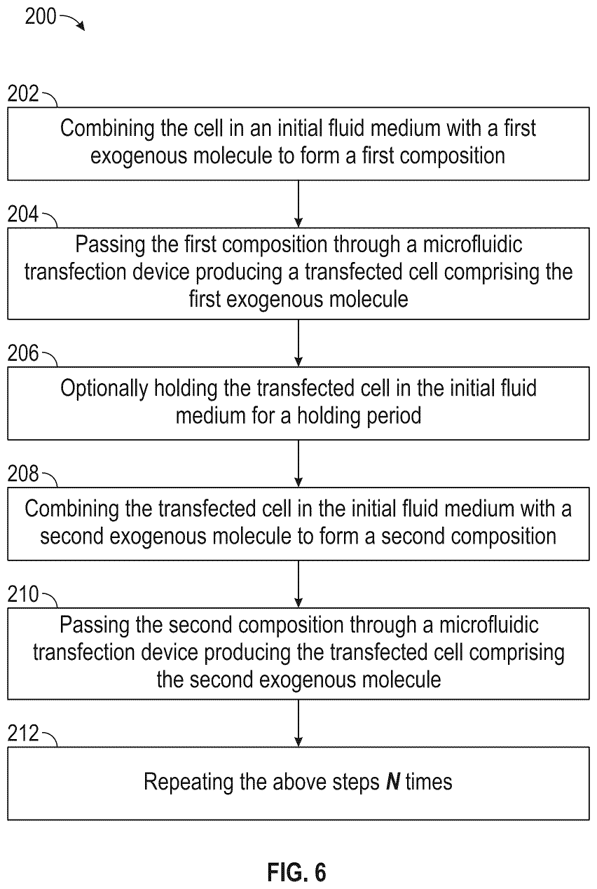

As illustrated in FIG. 6, the method 200 may comprise one or more steps. At block 202, the method includes combining the cell in an initial fluid medium with a first exogenous molecule to form a first composition.

As illustrated in FIG. 6, the second step 204 may comprise passing the first composition through a microfluidic transfection device producing a transfected cell comprising the first exogenous molecule. In an example, the transfection device may have various transfection parameters for an optimal first transfection event.

The transfection parameters may optimize the first transfection event. The first transfection event may result in optimal cell viability percentages and optimal transfection success percentages. In an example, the optimized first transfection event results in a cell viability percentage of about 50%, about 55%, about 60%, about 65%, about 70%, about 75%, about 80%, about 85%, or about 90%. In a further example, the transfection success rate in the optimized first transfection device is about 20% to about 40%.

As illustrated in FIG. 6, at step 206, the method 200 may include optionally holding the transfected cell in a holding chamber for a holding period. In an example, the transfected cell may be optionally held in the holding chamber for a sufficient period of time to allow the first population of transfected cells to recover from the first transfection event. In another example, the cells may be immediately injected with a second payload when they reach the holding chamber and have no holding period (e.g., no intervening culture step).

As illustrated in FIG. 6, at step 208, the method 200 may include combining the transfected cell in the holding chamber with a second exogenous molecule to form a second composition. In some examples, the second exogenous molecule may be a different exogenous molecule than the first exogenous molecule. In another example, the second exogenous molecule may be the same exogenous molecule as the first exogenous molecule.

As illustrated in FIG. 6, at step 210, the method 200 may include passing the second composition through a microfluidic transfection device producing the transfected cell comprising the second exogenous molecule (e.g., second transfection event). In an example, the transfection device may have various transfection parameters for an optimal second transfection event.

The second transfection event may result in optimal cell viability percentages and optimal transfection success percentages. In an example, the optimized first transfection event results in a cell viability percentage of about 50%, about 55%, about 60%, about 65%, about 70%, about 75%, about 80%, about 85%, or about 90%. In a further example, the transfection success rate in the optimized first transfection device is about 20% to about 40%.

As illustrated in FIG. 6, at step 212, the method may include repeating steps 202-210 N times to transfect the cell with N exogenous molecules producing N transfection events. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the exogenous molecule may be the same in some or all transfection events. In another example, the exogenous molecule may be different in all of the transfection events.

Also presented herein is a method for high throughput introduction of a plurality of exogenous molecules into a cell. The method may include passing the cell through N microfluidic processing cycles wherein N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Each cycle may include combining the cell in an initial fluid medium with an exogenous molecule to form a composition and passing the composition through a microfluidic transfection device, thereby introducing the exogenous molecule to the cell to form a transfected cell comprising the exogenous molecule. In an example, the exogenous molecule may comprise the same or a different molecule for each cycle. In another example, some or all of the cycles may include the same exogenous molecule. In a further example, all of the cycles may include a different exogenous molecule.

The transfection parameters of the transfection device may be optimized for each transfection event. In some examples, the transfection parameters may be optimized based on the size of the cell, the cell stiffness, adhesiveness, or the cell's FACS characteristics.

There may be a holding period after each complete cycle. The transfected cell may be optionally held in a holding chamber for a sufficient period of time (e.g., a holding period) to allow the first population of transfected cells to recover from the first transfection event. In some examples, the holding period can allow the cells sufficient time and conditions to express one or more nucleotide sequences. In another example, the cells may be immediately injected with a second payload when they reach the holding chamber and have no holding period (e.g., no intervening culture step).

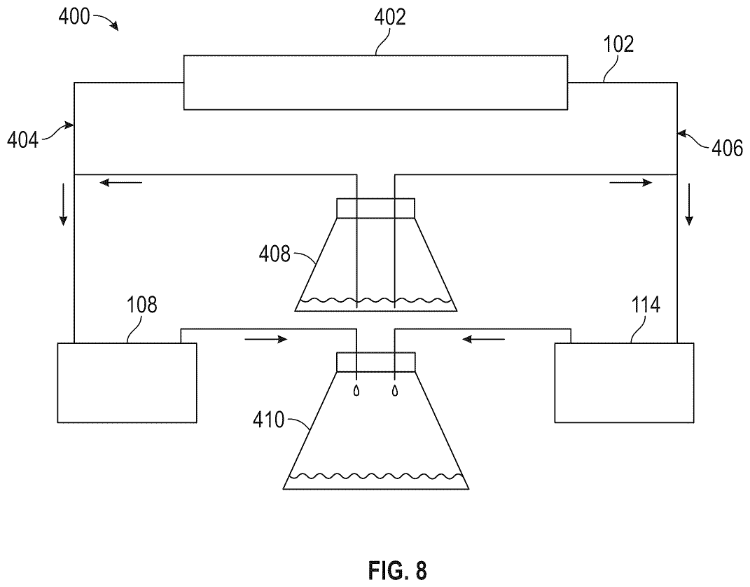

Also described herein is a system for high throughput introduction of an exogenous molecule into cells in a population of cells which are heterogenous. The system of FIG. 8 may be configured to transfect a heterogenous population of cells. The heterogenous cells may be sorted by a cell sorter into two or more groups (e.g., sub-populations). The groups may have different flow paths through different microfluidic transfection devices. The different microfluidic transfection devices may have different optimized transfection parameters depending on the group they are transfecting. The transfection parameters of each transfection device may be optimized based on the characteristics (e.g., size, stiffness, FACS characteristics, and/or other physical characteristics) of the group (i.e., sub-population) they are transfecting. The system may provide rapid transfection of two or more groups of cells at optimal viability and successful transfection rates. The different microfluidic transfection device may be arranged in parallel such that the first transfection event and the second transfection event occur at substantially the same time, transfecting both groups of cells with an exogenous molecule rapidly.

As illustrated in FIG. 8, the system 400 may include an initial fluid medium 102, a cell sorter 402, a first payload reservoir 106, a first microfluidic transfection device 108, and a second microfluidic transfection device 114. The system 400 may be operable to transfect a plurality of cells with at least one exogenous molecule. In an example, the first transfection device and the second transfection device may be arranged in parallel.

As illustrated in FIG. 8, the cell sorter 402 may be in fluid communication with the initial fluid medium 102. The cell sorter 402 may be configured to sort a heterogenous population of cells (i.e., plurality of cells). In an example, the cell sorter may be configured to sort the plurality of cells into a first sub-population of cells and a second sub-population of cells. The cell sorter 402 may sort the plurality of cells based on size, cell membrane stiffness, FACS characteristics, and other physical properties of the cells (e.g., shape, color, hardness, malleability, solubility, density, etc.). In an example, the FACS characteristics may include phenotypes, receptor types, and frequencies. It will be appreciated that the cell sorter 402 of this paragraph may be used in any other system or method described herein.

The first sub-population of cells may have a cell diameter below a first diameter cut-off value. The second sub-population of cells may have a cell diameter above a first diameter cut-off value. In another example, the first sub-population may have a cell stiffness below a stiffness cut-off value and the second sub-population may have a cell stiffness above a stiffness cut-off value. In a further example, the first sub-population may have a FACS characteristic, and the second sub-population of cells may have another FACS characteristic. In a further example, the cell sorter may be configured to sort the cells into a three, four, five, six, seven, eight, nine, or ten sub-populations. It will be appreciated that the sorting characteristics described in this paragraph may be used in any other system or method described herein.

As illustrated in FIG. 8, the initial fluid medium 102 may include a first flow path 404 and a second flow path 406. In an example, the first flow path 404 and the second flow path 406 may be arranged in parallel. The first flow path 404 may be operable to deliver the first sub-population of cells to the first microfluidic transfection device 108 from the cell sorter 402. The second flow path 406 may be operable to deliver the second sub-population of cells to the second microfluidic transfection device 114 from the cell sorter 402.

As illustrated in FIG. 8, the first payload reservoir 106 may be in fluid communication with the initial fluid medium 102 (i.e., the first flow path 404 and the second flow path 406). The first payload reservoir 106 may be operable to inject an exogenous molecule into the initial fluid medium 102 (i.e., the first flow path 404 and the second flow path 406). In an example, the first payload reservoir 106 may inject the first sub-population of cells with the exogenous molecule in the first flow path 404 forming a first composition. The first payload reservoir 106 may inject the second sub-population of cells with the exogenous molecule in the second flow path 406 forming a second composition.

As illustrated in FIG. 8, the first flow path 404 may be in fluid communication with the first microfluidic transfection device 108. The first composition may be passed through the first microfluidic transfection device 108. The first microfluidic transfection device 108 may include an inlet configured to receive the first composition from the first flow path 404, a transfection component configured to transfect the first composition with the exogenous molecule, thereby producing a first population of transfected cells (e.g., first transfection event), and an outlet configured to return the first population of transfected cells to the initial fluid medium 102.

The first sub-population of cells may have a successful transfection percentage of about 20%, about 25%, about 30%, about 35%, or about 40% after being transfected using the first and second microfluidic transfection device. In an example, the first sub-population of cells may have a successful transfection percentage of about 20% to about 40%. About 50%, about 55%, about 60%, about 65%, about 70%, about 75%, about 80%, about 85%, or about 90% of the cells may remain viable after being transfected using the first and second transfection device. In an example, about 50% to about 90% of the cells may remain viable after being transfected using the first transfection device.

As illustrated in FIG. 8, the second flow path 406 may be in fluid communication with the second microfluidic transfection device 114. The second composition may be passed through the second microfluidic transfection device 114. The second microfluidic transfection device 114 may include an inlet configured to receive the second composition from the second flow path 406, a transfection component configured to transfect the second composition with the exogenous molecule, thereby producing a second population of transfected cells (e.g., second transfection event), and an outlet configured to return the second population of transfected cells to the initial fluid medium 102.

The second sub-population of cells may have a successful transfection percentage of about 20%, about 25%, about 30%, about 35%, or about 40% after being transfected using the first transfection device. In an example, the second sub-population of cells may have a successful transfection percentage of about 20% to about 40%. About 50%, about 55%, about 60%, about 65%, about 70%, about 75%, about 80%, about 85%, or about 90% of the cells may remain viable after being transfected using the first transfection device. In an example, about 50% to about 90% of the cells may remain viable after being transfected using the second transfection device.

The transfection components of the first microfluidic transfection device 108 and the second microfluidic transfection device 114 may have various transfection parameters optimized for transfecting the first sub-population of cells and second sub-population of cells, respectively. In some examples, the transfection parameters of the first microfluidic transfection device 108 may be different from the transfection parameters of the second microfluidic transfection device 114, due to the differences in the first sub-population of cells and the second sub-population of cells. In an example, the transfection parameters of the first microfluidic transfection device 108 and second microfluidic transfection device 114 may be the same. In one example, the transfection parameters of the first microfluidic transfection device may be optimized for smaller cells while the transfection parameters for the second microfluidic transfection device may be optimized for larger cells. Similar optimizations of the transfection parameters may occur based on how the cells are initially sorter (e.g., the first microfluidic transfection device being optimized for stiffer cells while the second microfluidic transfection device is optimized for less stiff cells, etc.).

In one aspect, the first transfection event and the second transfection may occur at substantially the same time. In another aspect, the first transfection event and the second transfection event may occur at different times.

As illustrated in FIG. 8, the system 400 may have a holding chamber 110 in fluid communication with the initial fluid medium 102 (i.e., the first flow path 404 and the second flow path 406). The holding chamber 110 may be operable to receive the first population of transfected cells and the second population of transfected cells. In an example, the first and second populations of transfected cells may be optionally held in the holding chamber 110 for a sufficient period of time (e.g., a holding period) to allow the first and second populations of transfected cells to recover from the first transfection event (e.g., the transfection of the first sub-population of cells and the second sub-population of cells with the exogenous molecule). In some examples, the holding period can allow the cells sufficient time and conditions to express one or more nucleotide sequences.

The system may include a flow rate supply. In an example, the flow rate supply may be any device or instrument that provides a velocity to the cells in the system. In one example, the flow rate supply may be a pressure supply to apply a pressure of about 5 psi to about 100 psi to the system to move the cells through the system. The pressure supply may comprise a regulator having multiple control systems. In an example, the regulator may provide pressure at locations where the cells need to move. The regulator may supply pressure at a first location (e.g., the first flow path 404) to move the first sub-population of cells from the cell sorter 402 through the first microfluidic transfection device 108 device to the holding chamber 110. The regulator may supply a pressure at a second location (e.g., the second flow path 406) to move the second sub-population of cells from the cell sorter 402 through the second microfluidic transfection device 114 to the holding chamber 110.

In an aspect, the system may include multiple pressure supplies. Each pressure supply may have a regulator. In an example, a first pressure supply may have a regulator that may supply a pressure at a first location (e.g., the first flow path 404) to move the first sub-population of cells from the cell sorter 402 through the first microfluidic transfection device 108 device to the holding chamber 110. A second pressure supply may have a regulator that may supply a pressure at a second location (e.g., the second flow path 406) to move the second sub-population of cells from the cell sorter 402 through the second microfluidic transfection device 114 to the holding chamber 110.

A third pressure supply may supply a pressure to the holding chamber 110 to move the first population of transfected cells and the second population of transfected cells back to the cell sorter 402. The system 400 may be used to sort the cells and transfect the cells NV times with N different exogenous molecules. In an example N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30. In an example, the cells are sorted between each new set of transfection events and the microfluidic transfection devices are tuned between each new set of transfection events. In some examples, each of the N transfection events may transfect a sub-population of cells with a different exogenous molecule. In other examples, each transfection event may comprise the same exogenous molecule. In further examples, some transfection events may comprise the same exogenous molecule and other transfection events may comprise different exogenous molecules.

In one aspect, the system of FIG. 5 may be connected with the system of FIG. 8 to transfect the cells using the system of FIG. 5 first and then transfect the cells with the system of FIG. 8. In another aspect, the system of FIG. 8 may transfect the cells first and then the system of FIG. 5 may transfect the cells.

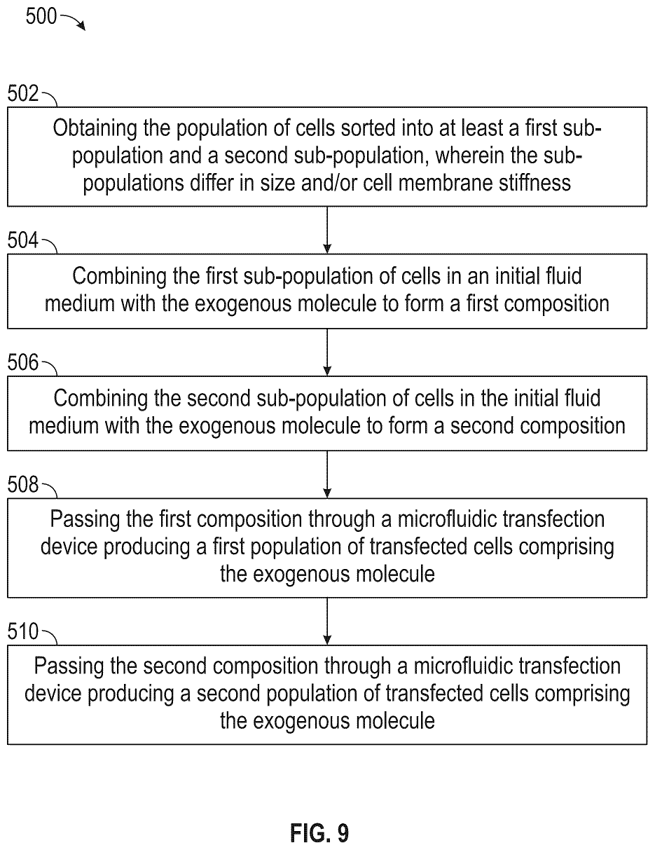

Further provided herein is a method for high throughput introduction of an exogenous molecule into cells in a population of cells which are heterogenous. The method of FIG. 9 may be configured to transfect a heterogenous population of cells. The heterogenous cells may be sorted by a cell sorter into two or more groups (e.g., sub-populations). The groups may have different flow paths through different microfluidic transfection devices. The different microfluidic transfection devices may have different optimized transfection parameters depending on the group they are transfecting. The transfection parameters of each transfection device may be optimized based on the characteristics (e.g., size, stiffness, FACS characteristics, and/or other physical properties of the cells as described herein) of the group (i.e., sub-population) they are transfecting. The method may provide rapid transfection of two or more groups of cells at optimal viability and successful transfection rates. The different microfluidic transfection devices may be arranged in parallel such that the first transfection event and the second transfection event occur at substantially the same time, transfecting both groups of cells with an exogenous molecule rapidly.

As illustrated in FIG. 9, the method 500 may comprise one or more steps. At a first step 502, the method may include obtaining the population of cells sorted into at least a first sub-population and a second sub-population. The first sub-population and the second sub-population may be differentiated by size, cell membrane stiffness, FACS characteristics, and/or other physical properties of the cells as provided herein. A flow rate may be provided to the first sub-population of cells to move the first sub-population cells through a first flow path of an initial fluid medium. A flow rate may be provided to the second sub-population of cells to the move the second sub-population of cells through a second flow path of an initial fluid medium. In an example, the first flow path may be in fluid communication with a first microfluidic transfection device. The second flow path may be in fluid communication with a second microfluidic transfection device.

As illustrated in FIG. 9, at a second step 504, the method 500 may include combining the first sub-population of cells in the initial fluid medium (e.g., first flow path) with the exogenous molecule to form a first composition. At a third step 506, the method 500 may include combining the second sub-population of cells in the initial fluid medium (e.g., second flow path) with the exogenous molecule to form a second composition. The exogenous molecule may be stored in a payload reservoir. The payload reservoir may provide the first sub-population of cells and the second sub-population of cells with the exogenous molecule to form the first composition and the second composition.

As illustrated in FIG. 9, at a fourth step 508, the method may include passing the first composition through a first microfluidic transfection device. In an example, the first microfluidic transfection device may have various transfection parameters optimized for transfecting the first sub-population of cells with the exogenous molecule. The first transfection event may result in optimal cell viability percentages and optimal transfection success percentages in the first sub-population of cells. In an example, the optimized first transfection event results in a cell viability percentage of about 50%, about 55%, about 60%, about 65%, about 70%, about 75%, about 80%, about 85%, or about 90%. In a further example, the transfection success rate in the optimized first transfection device is about 20% to about 40%.

As illustrated in FIG. 9, at a fifth step 510, the method may include passing the second composition through a second microfluidic transfection device, thereby producing a second population of transfected cells comprising the exogenous molecule. In an example, the second transfection device may have various transfection parameters optimized for transfecting the second sub-population of cells with the exogenous molecule. The transfection event may result in optimal cell viability percentages and optimal transfection success percentages in the second sub-population of cells. In an example, the optimized transfection event results in a cell viability percentage of about 50%, about 55%, about 60%, about 65%, about 70%, about 75%, about 80%, about 85%, or about 90%. In a further example, the transfection success rate in the optimized second microfluidic transfection device is about 20% to about 40%.

In one aspect, the transfection of the first sub-population of cells and the transfection of the second sub-population of cells may occur substantially simultaneously in the first microfluidic transfection device and the second microfluidic transfection device.

The method 500 may include optionally holding the first population of transfected cells and the second population of transfected cells in a holding chamber for a holding period. In an example, the first population of transfected cells and the second population of transfected cells may be optionally held in the holding chamber for a sufficient period of time (e.g., a holding period) to allow the first population and second population of transfected cells to recover from the transfection events. In some examples, the holding period can allow the cells sufficient time and conditions to express one or more nucleotide sequences.

The method may include repeating steps 502-510 N times to transfect the cells with N exogenous molecules in N transfection events. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, a different exogenous molecule may be transfected into each sub-population in each transfection event. In other examples, some or all of transfection events may transfect the same exogenous molecule into the first and second sub-populations of cells.

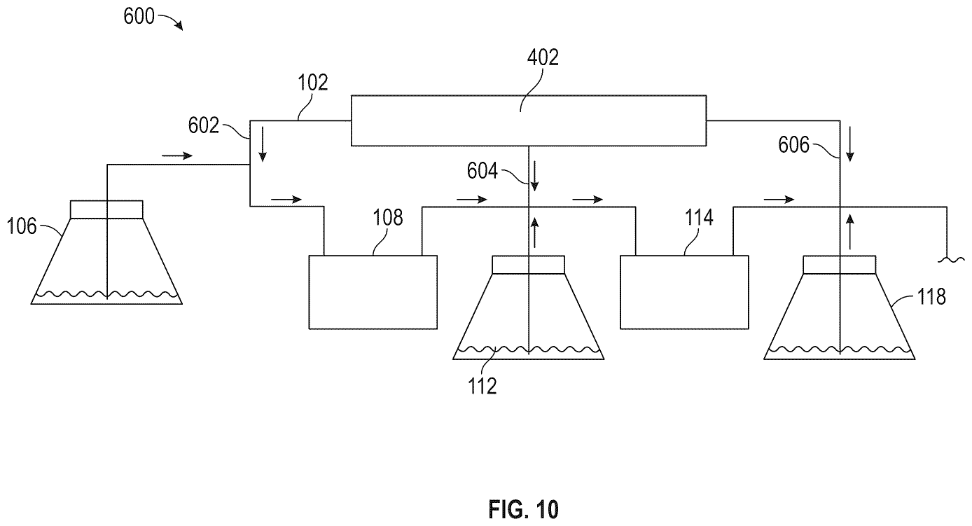

Also provided herein is a system 600 for transfecting a plurality of cells with a plurality of exogenous molecules. The system, as illustrated in FIG. 10, may be configured to sort the cells into at least two groups (i.e., sub-populations). The system may be operable to conduct two or more transfection events sequentially. By transfecting the cells sequentially, groups of cells may be transfected quickly while retaining the successful transfection and viability rates of methods known in the art. The system may be advantageous because it may be capable of successfully transfecting a heterogenous population of cells quickly without having to remove the cells from the system.

As illustrated in FIG. 10, the system 600 may include a cell sorter 402, an initial fluid medium 102, a first payload reservoir 106, a first microfluidic transfection device 108, a second payload reservoir 112, a second microfluidic transfection device 114, and a third payload reservoir 118. The system may be operable to transfect a plurality of cells with at least one exogenous molecule. The first transfection device and the second transfection device may be arranged in series.

As illustrated in FIG. 10, the cell sorter 402 may be in fluid communication with the initial fluid medium 102. The cell sorter may be configured to sort a heterogenous population of cells (i.e., plurality of cells). In an example, the cell sorter may be configured to sort the plurality of cells into a first sub-population of cells, a second sub-population of cells, and a third sub-population of cells. The cell sorter may sort the plurality of cells based on size, cell membrane stiffness, FACS characteristics, and/or other physical characteristics of the cells. In an example, the FACS characteristics may include phenotypes, receptor types, and frequencies.

The first sub-population of cells may be moved through a first flow path 602 (e.g., from the cell sorter 402 to a position in front of the first microfluidic transfection device 108) of the initial fluid medium 102 by supplying a flow rate using a flow rate supply to provide a cell velocity to the cells. The second sub-population of cells may be moved through a second flow path 604 (e.g., from the cell sorter 402 to a position behind the first microfluidic transfection device 108 and in front of the second microfluidic transfection device 114) by supplying a flow rate using a flow rate supply. The third sub-population of cells may be moved through a third flow path 606 (e.g., from the cell sorter 402 to a position behind the second microfluidic transfection device 114) of the initial fluid medium 102 by supplying a flow rate using a flow rate supply.

As illustrated in FIG. 10, the first payload reservoir 106 may be in fluid communication with the first flow path 602 of the initial fluid medium 102. The first payload reservoir 106 may contain a first exogenous molecule to be transfected into the first sub-population of cells. The first payload reservoir 106 may inject the exogenous molecule into the initial fluid medium 102 (e.g., the first flow path 602) by using a flow rate supply. When the first exogenous molecule is delivered to the initial fluid medium 102, the first exogenous molecule and the first sub-population of cells may form a first composition.

As illustrated in FIG. 10, the first composition may be passed through a first microfluidic transfection device 108. The first microfluidic transfection device 108 may include an inlet configured to receive the first composition, a transfection component configured to transfect the cell or cells with the first exogenous molecule, thereby producing a first population of transfected cells, and an outlet configured to return the first population of transfected cells to the initial fluid medium 102. In an example, the first population of transfected cells may comprise the first exogenous molecule. The transfection component of the first transfection device may have various transfection parameters optimized for transfecting the first sub-population of cells. The first sub-population of transfected cells may have a successful transfection percentage of about 20%, about 25%, about 30%, about 35%, or about 40% after being transfected using the first microfluidic transfection device. In an example, the cell or cells may have a successful transfection percentage of about 20% to about 40%. About 50%, about 55%, about 60%, about 65%, about 70%, about 75%, about 80%, about 85%, or about 90% of the cells may remain viable after being transfected using the first transfection device. In an example, about 50% to about 90% of the cells may remain viable after being transfected using the first microfluidic transfection device.

As illustrated in FIG. 10, after the first sub-population of cells have been transfected by the first microfluidic transfection device 108, the first population of transfected cells are returned to the initial fluid medium 102 via the outlet in the first microfluidic transfection device 108.

The first population of transfected cells may be optionally held in the initial fluid medium 102 for a sufficient period of time (e.g., a holding period) to allow the first population of transfected cells to recover from the first transfection event. In some examples, the holding period can allow the cells sufficient time and conditions to express one or more nucleotide sequences. In another example, the first population of transfected cells may have no holding period.

As illustrated in FIG. 10, the second sub-population of cells may be combined with the first population of cells in the initial fluid medium 102, forming a heterogenous cell product. The second sub-population of cells may flow through the second flow path 604 of the initial fluid medium 102 to combine with the first population of transfected cells.

As illustrated in FIG. 10, the second payload reservoir 112 may be in fluid communication with the initial fluid medium 102 at a location before the second microfluidic transfection device. The second payload reservoir may provide a second exogenous molecule to the first population of transfected cells and the second sub-population of cells. In an example, the second exogenous molecule, the first population of transfected cells, and the second sub-population of cells may be combined in the initial fluid medium to form a second composition. In one example, the second exogenous molecule may be a different exogenous molecule from the first exogenous molecule. In another example, the first exogenous molecule and the second exogenous molecule may be the same exogenous molecule. A flow rate supply may move the second composition through the initial fluid medium 102 and through the second microfluidic transfection device 114 by providing a velocity to the cells.

As illustrated in FIG. 10, the second composition may be passed through a second microfluidic transfection device 114. The second microfluidic transfection device 114 may include an inlet configured to receive the second composition, a transfection component configured to transfect the second composition with the second exogenous molecule, thereby producing a second population of transfected cells, and an outlet configured to return the second population of transfected cells to the initial fluid medium 102. In an example, the second population of transfected cells may comprise the first sub-population of cells and the second sub-population of cells transfected with the same exogenous molecule (e.g., when the first exogenous molecule and the second exogenous molecule are the same exogenous molecule). In another example, the second population of transfected cells may comprise the first sub-population of cells transfected with the first exogenous molecule and the second sub-population of cells transfected with the second exogenous molecule (e.g., when the first exogenous molecule and the second exogenous molecule are different exogenous molecules).

In one aspect, the cells may have a successful transfection percentage of about 20%, about 25%, about 30%, about 35%, or about 40% after being transfected using the second microfluidic transfection device 114. In an example, the cells may have a successful transfection percentage of about 20% to about 40% using the second microfluidic transfection device 114. About 50%, about 55%, about 60%, about 65%, about 70%, about 75%, about 80%, about 85%, or about 90% of the cells may remain viable after being transfected using the second microfluidic transfection device 114. In an example, about 50% to about 90% of the cells may remain viable after being transfected using the second microfluidic transfection device 114.

The transfection component of the second microfluidic transfection device 114 may have various transfection parameters optimized for transfecting the second sub-population of cells. The transfection parameters may depend on the characteristics of the second sub-population of cells (e.g., size, stiffness, FACS characteristics, and/or other physical characteristics).

As illustrated in FIG. 10, after the second sub-population of cells and/or the first population of transfected cells have been transfected by the second microfluidic transfection device 114, the second population of transfected cells are returned to the initial fluid medium 102 via the outlet in the second microfluidic transfection device 114.

The second population of transfected cells may be optionally held in the initial fluid medium 102 for a sufficient period of time (e.g., a holding period) to allow the second population of transfected cells to recover from the second transfection event. In some examples, the holding period can allow the cells sufficient time and conditions to express one or more nucleotide sequences. In another example, the cells may be immediately injected with a third payload in the initial fluid medium 102 and have no holding period (e.g., no intervening culture step).

As illustrated in FIG. 10, the third sub-population of cells may be moved into the initial fluid medium (e.g., through the third flow path 606) and combined with the second population of transfected cells.

As illustrated in FIG. 10, the third payload reservoir 118 may be in fluid communication with the initial fluid medium 102 at a location after the second microfluidic transfection device 114. The third payload reservoir may provide a third exogenous molecule to the second population of transfected cells and the third sub-population of cells in the initial fluid medium 102. In an example, the third exogenous molecule, the second population of transfected cells, and the third sub-population of cells may be combined in the initial fluid medium to form a third composition. In one example, the third exogenous molecule may be a different exogenous molecule from the first exogenous molecule and/or second exogenous molecule. In another example, the first exogenous molecule, second exogenous molecule, and third exogenous molecule may be the same exogenous molecule. In a further example, the first exogenous molecule may be the same as the second exogenous molecule but different than the third exogenous molecule. In another example, the second exogenous molecule and the third exogenous molecule may be the same exogenous molecule, and the first exogenous molecule may be different from the second exogenous molecule and the third exogenous molecule.

The third composition may be passed through a third microfluidic transfection device optimized for transfection of the third sub-population of cells, thereby producing a third composition of transfected cells comprising the first sub-population of cells transfected with the first exogenous molecule, the second sub-population of cells transfected with the second exogenous molecule, and the third sub-population of cells transfected with the third exogenous molecule.

The system may include a flow rate supply. In an example, the flow rate supply may be any device or instrument that provides a velocity to the cells in the system. In one example, the flow rate supply may be a pressure supply to apply a pressure of about 5 psi to about 100 psi to the system to move the cells between components. The pressure supply may comprise a regulator having multiple control systems. In an example, the regulator may provide pressure at locations where the cells need to move. The regulator may provide a pressure at a first location (e.g., at the cell sorter 402 before the first flow path 602) to move the first sub-population of cells through the first flow path 602, through the first exogenous molecule injection point, and through the first microfluidic transfection device 108 to a position connecting with the second flow path 604. The regulator may provide a pressure at a second location (e.g., at the cell sorter 402 before the second flow path 604) to move the second sub-population of cells through the second flow path 604, through the second exogenous molecule injection point, and then move both the first population of transfected cells and the second sub-population of cells through the second microfluidic transfection device 114. The regulator may provide a pressure at a third location (e.g., at the cell sorter 402 before the third flow path 606) to move the third population of cells through the third flow path 606, though the third exogenous molecule injection point, and then move both the second population of transfected cells and the third sub-population of cells through the third microfluidic transfection device.

In another aspect, the system may include multiple pressure supplies. Each pressure supply may have a regulator. In an example, a first pressure supply may have a regulator that may supply a pressure at a first location (e.g., at the cell sorter 402 before the first flow path 602) to move the first sub-population of cells through the first flow path 602, through the first exogenous molecule injection point, and through the first microfluidic transfection device 108 to a position connecting with the second flow path 604. A second pressure supply may have a regulator that may supply a pressure at a second location (e.g., at the cell sorter 402 before the second flow path 604) to move the second sub-population of cells through the second flow path 604, through the second exogenous molecule injection point, and then move both the first population of transfected cells and the second sub-population of cells through the second microfluidic transfection device 114. A third pressure supply may have a regulator that may supply a pressure at a third location (e.g., at the cell sorter 402 before the third flow path 606) to move the third population of cells through the third flow path 606, though the third exogenous molecule injection point, and then move both the second population of transfected cells and the third sub-population of cells through the third microfluidic transfection device.

The system may include N transfection devices and payload reservoirs configured to transfect N exogenous molecules into the cell or cells. The N holding chambers, transfection devices, and payload reservoirs may have the same structures, characteristics and properties as the first and second holding chambers, transfection devices, and payload reservoirs. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In an example, some or all of the transfection events may transfect the cells with the same exogenous molecule. In another example, the transfection events may transfect the cells with a different exogenous molecule.

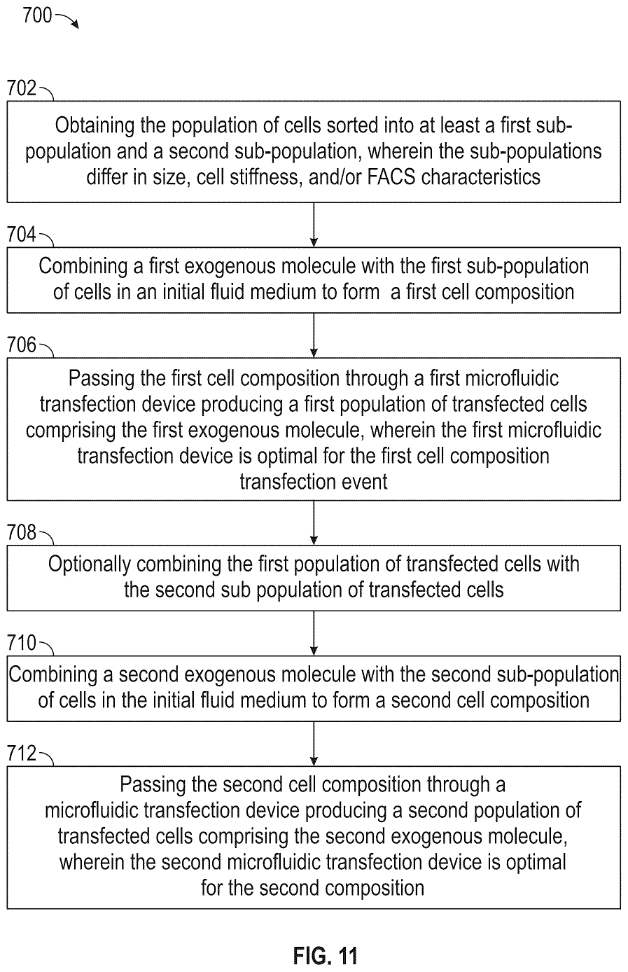

Further provided herein is a method for high throughput introduction of a plurality of exogenous molecules into a cell. The method 700, as illustrated in FIG. 11, may be configured to sort the cells into at least two groups (i.e., sub-populations). The system may be operable to conduct two or more transfection events sequentially. By transfecting the cells sequentially, groups of cells may be transfected quickly while retaining the successful transfection and viability rates of methods known in the art. The system may be advantageous because it may be capable of successfully transfecting a heterogenous population of cells quickly without having to remove the cells from the system.

As illustrated in FIG. 11, the method 700 may comprise one or more steps. At a first step 702, the method may include obtaining the population of cells sorted into at least a first sub-population and a second sub-population, wherein the sub-populations differ in size, cell stiffness, FACS characteristics, and/or physical characteristics. In an example, the population of cells to be sorted may be a heterogenous population of cells. The FACS characteristics may be one or more of phenotype, receptor type, and/or frequency. In an example, the first sub-population of cells may have a cell diameter less than a diameter cut-off value. The second-sub population of cells may have a diameter greater than a diameter cut-off value.

In an aspect, the method may include sorting the heterogenous population of cells into a first sub-population, a second sub-population, and a third sub-population. In an example, the heterogenous population of cells may be whole blood. Sorting the cells may include sorting the cells into at least a first sub-population having an average cell diameter of about 10 μm to about 12 μm (neutrophils), a second sub-population having an average cell diameter of about 12 μm to about 15 μm (lymphocytes), and a third sub-population having an average cell diameter of about 15 μm to about 30 μm (monocytes).

As illustrated in FIG. 11, at a second step 704, the method 700 may include combining a first exogenous molecule with the first-sub population of cells in an initial fluid medium to form a first cell composition. The first exogenous molecule may be injected into the initial fluid medium along a first flow path by a first payload reservoir. The first sub-population of cells may be moved through the first flow path to combine with the first exogenous molecule using a flow rate supply (e.g., pressure supply).

As illustrated in FIG. 11, at a third step 706, the method 700 may include passing the first cell composition through a microfluidic transfection device. Passing the first composition through the microfluidic transfection device may produce a first population of transfected cells comprising the first exogenous molecule. The transfection device may be optimally tuned for the first composition transfection event.

The method 700 may include optionally holding the first population of transfected cells in the initial fluid medium for a holding period. In an example, the first population of transfected cells may be optionally held in the initial fluid medium for a sufficient period of time (e.g., a holding period) to allow the first population of transfected cells to recover from the first transfection event. In some examples, the holding period can allow the cells sufficient time and conditions to express one or more nucleotide sequences. In another example, the first population of transfected cells may require no holding period before a second transfection event.

As illustrated in FIG. 11, at a fourth step 708, the method 700 may include optionally combining the first population of transfected cells with the second sub-population of cells. The second sub-population of cells may be moved from the cell sorter through the initial fluid medium along a second flow path to combine with the first population of transfected cells at a location in the initial fluid medium. The second sub-population of cells and the first population of transfected cells may be moved by supplying a flow rate using a flow rate supply (e.g., pressure supply).

As illustrated in FIG. 11, at a fifth step 710, the method 700 may include combining a second exogenous molecule with the second sub-population of cells, and optionally the first population of transfected cells, in the initial fluid medium to form a second cell composition. The second exogenous molecule may be injected into the initial fluid medium by a second payload reservoir. In some examples, the first exogenous molecule and the second exogenous molecule may be the same exogenous molecule. In another example, the first exogenous molecule and the second exogenous molecule may be different exogenous molecules.

As illustrated in FIG. 11, at a sixth step 712, the method 700 may include passing the second composition through a microfluidic transfection device. Passing the second composition through the microfluidic transfection device may produce a second population of transfected cells comprising the second exogenous molecule. The microfluidic transfection device may be optimally tuned for the second composition transfection event. In an example, the microfluidic transfection device may have various transfection parameters for an optimal second transfection event.

The method 700 may include optionally holding the second population of transfected cells in the initial fluid medium for a holding period. In an example, the second population of transfected cells may be optionally held in the initial fluid medium for a sufficient period of time (e.g., a holding period) to allow the second population of transfected cells to recover from the second transfection event. In some examples, the holding period can allow the cells sufficient time and conditions to express one or more nucleotide sequences. In another example, the second population of transfected cells may require no holding period.

In one aspect, the steps of the method 700 may be repeated with a third sub-population of cells to produce a third population of transfected cells. In another example, the method 700 may include repeating the steps of the method N times to transfect the sub-populations of cells with N exogenous molecules. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

Also presented herein is a method for high throughput introduction of a plurality of exogenous molecules into a heterogenous population of cells. The method may include passing the cell through N microfluidic processing cycles wherein N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Each cycle may include combining obtaining the population of cells sorted into at least a first sub-population and a second sub-population, combining a first exogenous molecule with the first sub-population of cells in an initial fluid medium to form a first cell composition, and passing the first cell composition through a microfluidic transfection device, thereby introducing the first exogenous molecule into the first sub-population of cells to form a first population of transfected cells comprising the first exogenous molecule. The cycle may further include combining a second exogenous molecule and optionally the first exogenous molecule with the second sub-population of cells and optionally the first population of transfected cells in the initial fluid medium to form a second cell composition, and passing the second cell composition through a microfluidic transfection device thereby introducing the second exogenous molecule and the optionally the first exogenous molecule into the second sub-population of cells and optionally the first population of transfected cells to form a second population of transfected cells comprising the second exogenous molecule and optionally the first exogenous molecule. The cycle may further include observing a holding period after each complete cycle or after each transfection event.

The transfection parameters of the transfection device may be tuned or optimized for each transfection event. In some examples, tuning the transfection device may include increasing or decreasing the gap size of the microfluidic transfection device based on the size, stiffness, FACS characteristics, or physical properties of the population of cells.

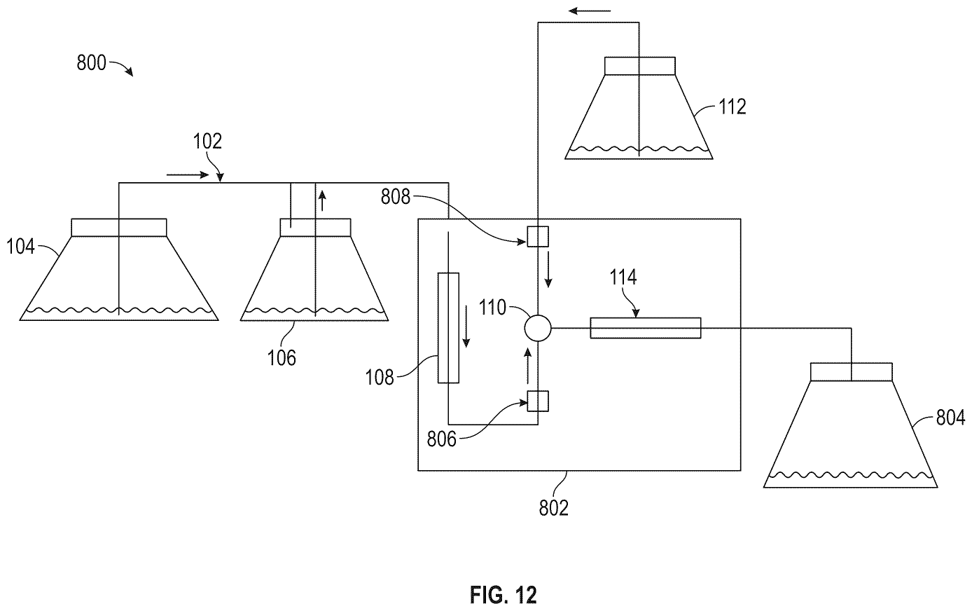

Further provided herein is a system for transfecting a plurality of cells with at least one exogenous molecule. The system 800 illustrated in FIG. 12 may be configured to sequentially transfect a plurality of cells. The sequential transfection can greatly reduce the time between successive transfection events compared to systems known in the art, while maintaining or improving the successful transfection rate and viability percentage of the cells.

As illustrated in FIG. 12, the system 800 may include a first reservoir 104, an initial fluid medium 102, a first payload reservoir 106, a microfluidic cassette 802, a second payload reservoir 112, and a collection reservoir 804.

As illustrated in FIG. 12, the first reservoir may be in fluid communication with the initial fluid medium 102. The first reservoir 104 may contain a plurality of cells. In an example, the plurality of cells may be homogenous. In another example, the plurality of cells may be heterogenous. The first reservoir 104 may be configured to provide the plurality of cells to the initial fluid medium 102. In an example, the plurality of cells may be moved from the first reservoir 104 to the initial fluid medium 102 by supplying a flow rate using a flow rate supply (e.g., pressure supply) to provide a cell velocity to the cells.

As illustrated in FIG. 12, the system 800 may include the first payload reservoir 106 in fluid communication with the initial fluid medium 102. The first payload reservoir 106 may be configured to inject a first exogenous molecule into the plurality of cells forming a first composition.

As illustrated in FIG. 12, the initial fluid medium 102 may be in fluid communication with a microfluidic cassette 802. The microfluidic cassette 802 may include a first microfluidic transfection device 108, a second microfluidic transfection device 114, and a holding chamber 110. The microfluidic cassette 802 may include a first inlet in fluid communication with the initial fluid medium 102, a second inlet in fluid communication with a second payload reservoir 112, and an outlet in fluid communication with the collection reservoir 804.

The microfluidic cassette 802 may include two or more valves. In an example, a first valve 806 may be located between the first microfluidic transfection device 108 and the holding chamber 110. A second valve 808 may be located between a second payload reservoir 112 in fluid communication with the second inlet of the microfluidic cassette 802. The first valve 806 may have an open state and a closed state. The second valve 808 may have an open state and a closed state. In the open state, the first and second valves may allow cells or molecules to travel through the valves. In the closed state, the first and second valves may block cells or molecules from traveling through the valves.

The first microfluidic transfection device 108 may be in fluid communication with the first inlet of the microfluidic cassette 802 and in fluid communication with the holding chamber 110 of the microfluidic cassette 802. The first microfluidic transfection device 108 may include an inlet configured to receive the first composition, a transfection component configured to transfect the plurality of cells with the first exogenous molecule, thereby producing a first population of transfected cells, and an outlet configured to return the first population of transfected cells to the holding chamber 110. In an example, the first population of transfected cells may comprise the first exogenous molecule.

The cell or cells may have a successful transfection percentage of about 20%, about 25%, about 30%, about 35%, or about 40% after being transfected using the first transfection device. In an example, the cell or cells may have a successful transfection percentage of about 20% to about 40%. About 50%, about 55%, about 60%, about 65%, about 70%, about 75%, about 80%, about 85%, or about 90% of the cells may remain viable after being transfected using the first transfection device. In an example, about 50% to about 90% of the cells may remain viable after being transfected using the first transfection device.

The expansion capacity of cell or cell population is further preserved after transfection using transfection device. In some aspects, the cell or cell population retains 20%, about 25%, about 30%, about 35%, about 40%, about 55%, about 60%, about 65%, about 70%, about 75%, or about 80% expansion capacity.

Further, transfection using transfection device disclosed herein preserves stemness of the cell or cell population. In one aspect, transfection using the transfection device preserve population of cell that are stem-like (Tscm). Stem-like cells can be quantified by identifying cell subsets, for example, T cell subsets, using methods known in the art. In certain aspects, after priming preserves stem-like cells by about 20%, about 25%, about 30%, about 35%, about 40%, about 55%, about 60%, about 65%, about 70%, about 75%, or about 80%. In further aspects, the cell or cell population retain a stemness of about 20%, about 25%, about 30%, about 35%, about 40%, about 55%, about 60%, about 65%, about 70%, about 75%, or about 80%. In one aspect, cells have a preserved stemness with about 50% cells in the population being Tscm population.

The cell or cells may further be cryopreserved. Cryopreservation can comprise any known method in the art. In one aspects, cell or cells are cooled at about 1° C./min during cryopreservation. Cryopreservation temperature is about −80° C. to about −180° C., for example about −125° C. to about −140° C. Cryopreserved cells can be transferred to liquid nitrogen prior to thawing for use. Cryopreservation can also be done using a controlled-rate freezer. In certain aspects, cryopreserved cells are thawed at a temperature of about 25° C. to about 40° C., preferably to a temperature of about 37° C. In further aspects, the cell or cells retain capacity to expand post-thaw. Capacity to expand can be determined by measuring the total number of live cells or percentage of live cells using method known in the art. In one example, automatic cell counter can be used to measure live or dead cell number. Post-thaw, about 50%, about 55%, about 60%, about 65%, about 70%, about 75%, about 80%, about 85%, or about 90% of the cells may remain viable. In an example, about 50% to about 90% of the cells may remain viable after thaw.

The transfection component of the first microfluidic transfection device 108 may have various transfection parameters optimized for transfecting the plurality of cells depending on the characteristics of the plurality of cells (e.g., size, stiffness, FACS characteristics, and/or physical characteristics).

As illustrated in FIG. 12, the first valve 806 may be placed in an open state to move the first population of transfected cells to the holding chamber 110. The first population of transfected cells may be moved from the first microfluidic transfection device 108 to the holding chamber 110 by supplying a flow rate using the flow rate supply (e.g., pressure supply) to supply a cell velocity to the cells.

As illustrated in FIG. 12, the holding chamber 110 may be in fluid communication with the first microfluidic transfection device 108, the second microfluidic transfection device 114, and the second inlet of the microfluidic cassette 802. The holding chamber 110 may be operable to receive the first population of transfected cells from the first microfluidic transfection device 108. In an example, the first population of transfected cells may be optionally held in the holding chamber 110 for a sufficient period of time (e.g., a holding period) to allow the first population of transfected cells to recover from the first transfection event. In some examples, the holding period can allow the cells sufficient time and conditions to express one or more nucleotide sequences. In another example, the first population of transfected cells may be injected with a second exogenous molecule and passed through the second microfluidic transfection device 114 immediately (e.g., no intervening culture step).

As illustrated in FIG. 12, the second payload reservoir 112 may supply a second exogenous molecule to the holding chamber 110 through the second inlet of the microfluidic cassette 802 when the second valve 808 is in an open state by supplying a flow rate through a flow rate supply (e.g., pressure supply). The second exogenous molecule and the first population of transfected cells may form a second composition.

In another aspect, the second payload reservoir 112 may also include a second population of cells to be moved to the holding chamber 110. The second payload reservoir 112 may inject the second population of cells and a second exogenous molecule into the holding chamber 110 when the second valve 808 is in an open state, forming a second composition with the first population of transfected cells. The second population of cells may have different characteristics from the plurality of cells transfected with the first exogenous molecule. The second population of cells may be of a different size, stiffness, have different FACS characteristics, and/or have different physical properties. The first plurality of cells and second population of cells may comprise a heterogenous population of cells that are sorted (e.g., by a cell sorter) prior to the first plurality of cells being placed in the first reservoir 104.

As illustrated in FIG. 12, the second composition may move from the holding chamber 110 to the second microfluidic transfection device 114. The second microfluidic transfection device 114 may include an inlet configured to receive the second composition from the holding chamber 110, a transfection component configured to transfect the second composition with the second exogenous molecule, thereby producing a second population of transfected cells, and an outlet in fluid communication with the outlet of the microfluidic cassette 802 configured to move the second population of transfected cells to the collection reservoir 804.

The transfection component of the second microfluidic transfection device 114 may have various transfection parameters optimized for transfecting the cell or cells, depending on the characteristics of the cells in the second composition (e.g., cell size, stiffness, FACS characteristics, and/or other physical properties).

As illustrated in FIG. 12, the collection reservoir 804 may be operable to receive the second population of transfected cells from the microfluidic cassette 802. The collection reservoir 804 may be in fluid communication with the outlet of the microfluidic cassette 802.

The system may include a flow rate supply. In an example, the flow rate supply may be any device or instrument that provides a velocity to the cells in the system 800. In one example, the flow rate supply may be a pressure supply to apply a pressure of about 5 psi to about 100 psi to the system to move the cells between components.

The pressure supply may comprise a regulator having multiple control systems. In an example, the regulator may provide pressure at locations where the cells need to move. The regulator may supply pressure at a first location (e.g., the first reservoir 104) to move the cell or cells from the first reservoir 104 through the initial fluid medium 102, into the microfluidic cassette 802, through the first microfluidic transfection device 108, and into the holding chamber 110. The regulator may supply a pressure at a second location (e.g., the second payload reservoir 112) to move the second exogenous molecule from the second payload reservoir 112 through the second inlet of the microfluidic cassette 802 and into the holding chamber 110. The regulator may supply a pressure at a third location (e.g., the holding chamber 110) to move the second composition through the second microfluidic transfection device 114, through the outlet of the microfluidic cassette 802, and into the collection reservoir 804.

In another aspect, the system may include multiple pressure supplies. Each pressure supply may have a regulator. In an example, a first pressure supply may have a regulator that may supply a pressure at a first location (e.g., the first reservoir 104) to move the cell or cells from the first reservoir 104 through the initial fluid medium 102, into the microfluidic cassette 802, through the first microfluidic transfection device 108, and into the holding chamber 110. A second pressure supply may have a regulator that may supply a pressure at a second location (e.g., the second payload reservoir 112) to move the second exogenous molecule from the second payload reservoir 112 through the second inlet of the microfluidic cassette 802 and into the holding chamber 110. A third pressure supply may have a regulator that may supply a pressure at a third location (e.g., the holding chamber 110) to move the second composition through the second microfluidic transfection device 114, through the outlet of the microfluidic cassette 802, and into the collection reservoir 804.

Also described herein is a method for high throughput introduction of a plurality of exogenous molecules to a plurality of cells. The method 900 illustrated in FIG. 9 may be configured to sequentially transfect a plurality of cells. The sequential transfection can greatly reduce the time between successive transfection events compared to methods known in the art, while maintaining or improving the successful transfection rate and viability percentage of the cells.

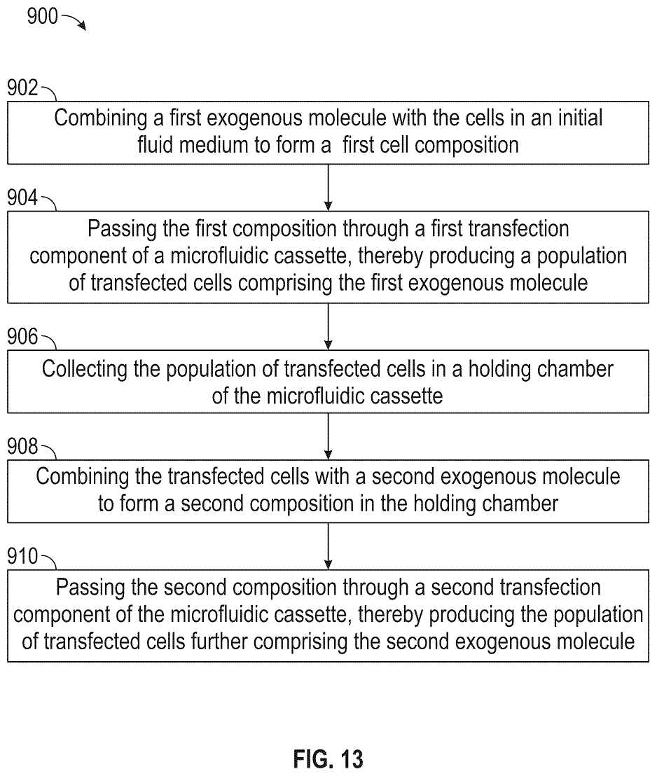

As illustrated in FIG. 13, the method 900 may begin at a first step 902. At the first step 902, the method 900 may include combining a first exogenous molecule with the plurality of cells in an initial fluid medium to form a first cell composition. The first exogenous molecule may be provided by a first payload reservoir.

As illustrated in FIG. 13, at a second step 904, the method may include passing the first composition through a transfection component of a first microfluidic transfection device in a microfluidic cassette to produce a population of transfected cells comprising the first exogenous molecule. In an example, the transfection component of the first microfluidic transfection device may be tuned or optimized to transfect the plurality of cells with the first exogenous molecule. Tuning the transfection component may include increasing or decreasing the gap size or adjusting other transfection parameters.

As illustrated in FIG. 13, at a third step 906, the method 900 may include collecting the population of transfected cells in a holding chamber of the microfluidic cassette. The population of transfected cells may be held in the holding chamber for a sufficient period of time to allow recovery of the population of transfected cells.

As illustrated in FIG. 13, at a fourth step 908, the method 900 may include combining the transfected cells with a second exogenous molecule to form a second composition. The second exogenous molecule may be provided by a second payload reservoir in fluid communication with the holding chamber. In another example, the second payload reservoir may provide a second population of cells and a second exogenous molecule to the holding chamber forming a second composition with the transfected cells.

As illustrated in FIG. 13, at a fifth step 910, the method 900 may include passing the second composition through a transfection component of a second microfluidic transfection device. Passing the second composition through the transfection component of the second microfluidic transfection device may produce a population of transfected cells comprising the second exogenous molecule. The transfection component of the second microfluidic transfection device may be tuned or optimized for transfection of the second composition with the second exogenous molecule and the first exogenous molecule. In an example, the tuning or optimizing the transfection component may include increasing or decreasing the gap size of the transfection component or adjusting other transfection parameters.

The population of transfected cells comprising the first and second exogenous molecules may be collected in a collection reservoir.

In another example, the method 900 may include repeating the steps of the method N times to transfect the sub-populations of cells with N exogenous molecules. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

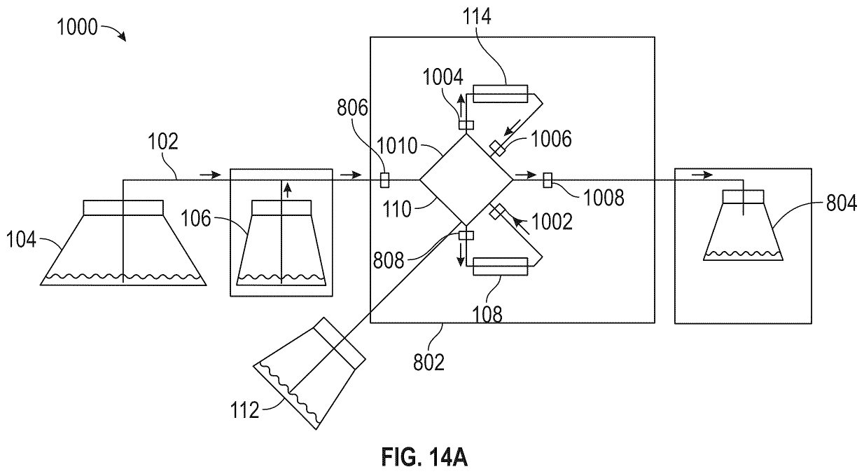

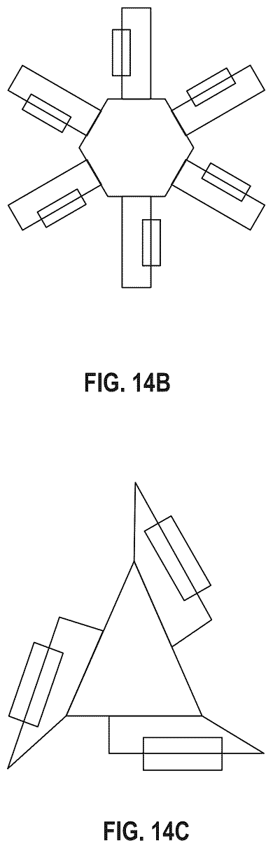

Further provided herein is a system for transfecting a plurality of cells with at least one exogenous molecule. The system 1000 illustrated in FIG. 14A may be configured to transfect a plurality of cells with at least one exogenous molecule. The system 1000 of FIG. 14A can be configured to sequentially transfect a plurality of cells with multiple exogenous molecules quickly, while maintaining the same or better rates of successful transfection and viability compared to other systems known in the art. The system of FIG. 14A is configured to produce multiple transfection events within a short amount of time (e.g., sequential transfection events may occur within the same hour).

The system of FIG. 12 may be alternatively configured as illustrated in FIG. 14A. The system 1000 may include the first reservoir 104, the first payload reservoir 106, the holding chamber 110, the microfluidic cassette 802 comprising the first microfluidic transfection device 108, the second microfluidic transfection device 114, the first valve 806, the second valve 808, and the collection reservoir 804.

As illustrated in FIGS. 14B and 14C, the microfluidic cassette may include more than two microfluidic transfection devices. The microfluidic cassette may include three, four, five, six, seven, eight, nine, or ten microfluidic transfection devices. Each microfluidic transfection device may be tuned or optimized for different cell types or exogenous molecule types as discussed above.

As illustrated in FIG. 14A, the microfluidic transfection device may have a plurality of valves. A first valve 806 may be located on the initial fluid medium 102 and in fluid communication with the first reservoir 104, the first inlet of the microfluidic cassette 802, and the holding chamber 110. The first valve 806 may have an open state and a closed state. A second valve 808 may be located between the holding chamber 110 and the first microfluidic transfection device 108. The second valve 808 may have an open state and a closed state. A third valve 1002 may be located between the first microfluidic transfection device 108 and the holding chamber 110. A fourth valve 1004 may be located between the holding chamber 110 and the second microfluidic transfection device 114. A fifth valve 1006 may be located between the second microfluidic transfection device 114 and the holding chamber 110. A sixth valve 1008 may be located on a microfluidic cassette outlet path between the holding chamber 110 and the microfluidic cassette outlet. The first, second, third, fourth, fifth, and sixth valves may be control valves having an open state and a closed state. One or more of the valves may be opened to direct the cells to a desired location. In an example, only one valve is in an open state at a time. In another example, two valves may be in an open state at one time. The system 1000 may have more than six valves when the system 1000 has more than two microfluidic transfection devices.

The first valve 806 may be in an open state to allow the cells to enter the holding chamber 110 from the first inlet of the microfluidic cassette 802. Once the cells have entered the holding chamber 110 the first valve 806 may be placed in a closed state. In an example, the second valve 808 may be placed in an open state. A flow rate (e.g., via a flow rate supply such as a pressure supply 1010) may be supplied to the holding chamber 110 to move the cells to the inlet of the first microfluidic transfection device. When the second valve 808 is in an open state the cells may move through the first microfluidic transfection device 108 to produce a first population of transfected cells. The second valve 808 may be placed in a closed state after the cells have traveled through the first microfluidic transfection device 108. The third valve 1002 may then be placed in an open state to allow the first population of transfected cells to move from the first microfluidic transfection device 108 through the first transfection device outlet path back to the holding chamber 110. The third valve 1002 may then be placed in a closed state.

In one aspect, the system may not include the first payload reservoir 106. The cells may flow directly into the holding chamber 110 from the first reservoir 104 through the first valve 806 in an open state. The cells may be combined with a first exogenous molecule in the holding chamber 110 to form a first composition. The first exogenous molecule may be provided by the second payload reservoir 112.

Once the first population of transfected cells have returned to the holding chamber 110, the second payload reservoir 112 may supply the first population of transfected cells with a second exogenous molecule forming a second composition.

A flow rate (e.g., via a flow rate supply such as a pressure supply 1010) may be supplied to the holding chamber 110 to move the second composition to the inlet of the second microfluidic transfection device 114. When the fourth valve 1004 is in an open state the cells may move through the second microfluidic transfection device 114 to produce a second population of transfected cells. The fourth valve 1004 may be placed in a closed state after the second composition has traveled through the second microfluidic transfection device 114. The fifth valve 1006 may then be placed in an open state to allow the second population of transfected cells to move from the second microfluidic transfection device 114 back to the holding chamber 110. The fifth valve 1006 may then be placed in a closed state.

In one aspect, to remove the cells from the microfluidic cassette 802, a flow rate may be applied to the cells in the holding chamber and the sixth valve 1008 may be placed in an open state. The cells may then flow through the sixth valve 1008 and out of the microfluidic cassette 802 into the collection reservoir 804. In another aspect, the cells may be removed through the microfluidic cassette by supplying a flow rate to the holding chamber, placing the first valve 806 in an open state, and allowing the cells to flow back into the first reservoir 104.

As illustrated in FIG. 14A, after the cells are transfected with an exogenous molecule in one of the microfluidic transfection devices they may return to the holding chamber 110. The cells may be held in the holding chamber for a holding period as described above. In another example, the cells may be immediately injected with another exogenous molecule and then passed through another transfection device (e.g., no intervening culture step). The payload (e.g., exogenous molecule) may be replaced between each transfection event in the second payload reservoir 112. This process may be repeated N times with N microfluidic transfection devices and N exogenous molecules until the cells have been transfected a desired number of times. In some examples, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In an example, the exogenous molecules may be the same throughout some or all of the transfection events to produce a higher transfection percentage. In another example, the exogenous molecules may be different for each transfection event.

A single flow rate supply (e.g., pressure supply 1010) may be provide a flow rate (e.g., via a pressure) to the holding chamber 110 to move the cells through the first microfluidic transfection device 108 and the second microfluidic transfection device 114. The valves may be operable to direct the cells to the first microfluidic transfection device 108 or second microfluidic transfection device 114.

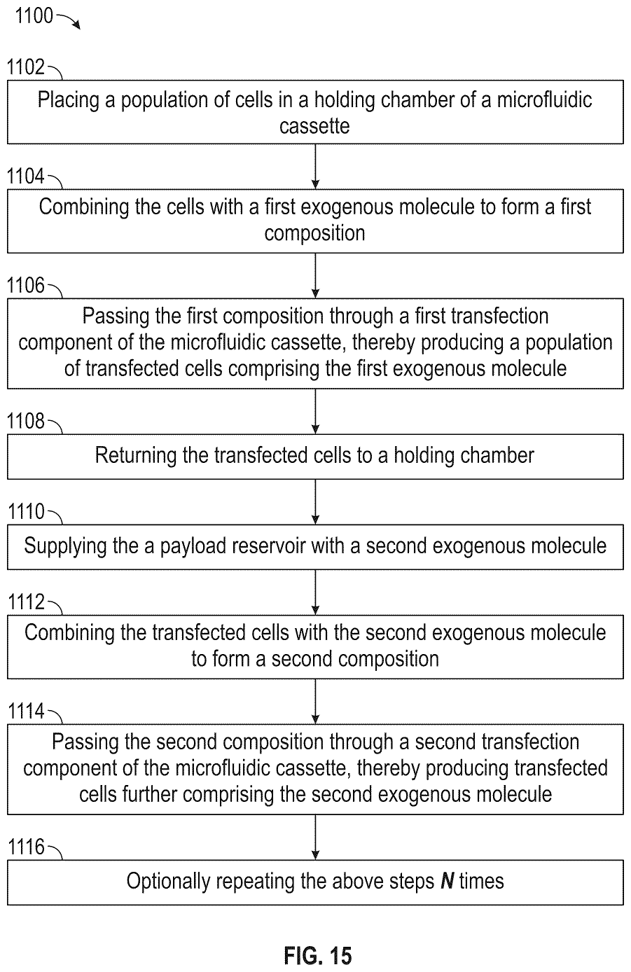

Also provided herein is a method for transfecting a plurality of cells with a plurality of exogenous molecules. The method, as illustrated in FIG. 15, may provide a sequential transfection of a plurality of cells with a plurality of exogenous molecules rapidly. The method may provide transfection of a plurality of cells with a plurality of exogenous molecules much quicker than methods known in the art, while maintaining the same or better successful transfection rates and viability percentages.

As illustrated in FIG. 15, at a first step 1102, the method 1100 may begin by placing the cells in a holding chamber of a microfluidic cassette. A first valve of the microfluidic cassette may be placed in an open state to allow the cells to flow into a first inlet of the holding chamber. The cells may be provided with a flow rate via a flow rate supply (e.g., a pressure supplied by a pressure supply). After the cells have all moved into the holding chamber, the first valve may be placed in a closed state.

As illustrated in FIG. 15, at a second step 1104, the method 1100 may include combining the cells with a first exogenous molecule to form a first composition. The first exogenous molecule may be selected from the exogenous molecules described above. The first exogenous molecule may be combined with the cells in the holding chamber by providing a flow rate (e.g., via a pressure from a pressure supply) to a payload reservoir to move the first exogenous molecule to the holding chamber. The first exogenous molecule can flow along a flow path to a second inlet of the holding chamber.

As illustrated in FIG. 15, at a third step 1106, the method 1100 may include passing the first composition through a first microfluidic transfection device of the microfluidic cassette. By passing the first composition through the first microfluidic transfection device, a first population of transfected cells comprising the first exogenous molecule may be produced. To pass the cells through the first microfluidic transfection device, a flow rate may be supplied to the holding chamber (e.g., a pressure via a pressure supply). A second valve may be placed in an open state on a flow path from the holding chamber to the first microfluidic transfection device to allow the first composition to enter the inlet of the first microfluidic transfection device. The flow rate may push the first composition through the microfluidic component of the first microfluidic transfection device and out the outlet of the first microfluidic transfection device. After the first composition has passed through the inlet of the first microfluidic transfection device, the second valve may be placed in a closed state. A third valve on a first transfection device outlet path may then be placed in an open state to allow the first population of transfected cells to move to the holding chamber through a third inlet of the holding chamber. In an example, the microfluidic component of the first microfluidic transfection device may be tuned or optimized for the transfection of the first composition. In some examples, tuning or optimizing the microfluidic transfection component may include increasing or decreasing the gap size.

As illustrated in FIG. 15, at a fourth step 1108, the method 1100 may include returning the first population of transfected cells to the holding chamber. In an example, the first population of transfected cells may be optionally held in the holding chamber for a sufficient period of time (e.g., a holding period) to allow the first population of transfected cells to recover from the first transfection event. In another example, the cells may be immediately injected with a second payload when they reach the holding chamber and have no holding period (e.g., no intervening culture step).

As illustrated in FIG. 15, at a fifth step 1110, the method 1100 may include suppling the payload reservoir with a second exogenous molecule. The payload reservoir may be easily reloaded with the second exogenous molecule. In another example, the payload reservoir may be removed and replaced with a new payload reservoir containing the second exogenous molecule.

As illustrated in FIG. 15, at a sixth step 1112, the method 1100 may include combining the population of transfected cells with the second exogenous molecule to form a second composition. A flow rate (e.g., via a pressure from a pressure supply) can be supplied the payload reservoir to move the second exogenous molecule from the payload reservoir to the holding chamber.

As illustrated in FIG. 15, at a seventh step 1114, the method 1100 may include passing the second composition through a second microfluidic transfection device of the microfluidic cassette. By passing the second composition through the second microfluidic transfection device, a second population of transfected cells is produced comprising the second exogenous molecule and the first exogenous molecule. A flow rate may be provided to the holding chamber to move the second composition through an inlet flow path of the second microfluidic transfection device. A fourth valve located on the inlet flow path of the second microfluidic device may be placed in an open state to allow the second composition to flow into the inlet of the second microfluidic transfection device. Once the second composition is fully into the microfluidic component of the second microfluidic transfection device, the fourth valve may be placed in a closed position. The second composition may then be passed through the microfluidic transfection component of the second microfluidic transfection device. In some examples, the microfluidic transfection component may be tuned or optimized for transfection of the second composition. In some examples, tuning or optimizing the transfection component may comprise increasing or decreasing the gap size or adjusting other transfection parameters. The second population of transfected cells may then be returned to the holding chamber. A fifth valve may be placed in an open state to allow the second population of transfected cells to flow to from the outlet of the second microfluidic transfection device along a second microfluidic transfection device outlet path and into the holding chamber.

The second population of transfected cells may be optionally held in the holding chamber for a sufficient period of time (e.g., a holding period) to allow the second population of transfected cells to recover from the second transfection event. In some examples, the holding period can allow the cells sufficient time and conditions to express one or more nucleotide sequences.

At an eighth step 1116, the method 1100 may include repeating the steps N times. N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. The microfluidic cassette may have N microfluidic transfection devices and two times N valves. The method may be repeated to produce a desired number of transfection events. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

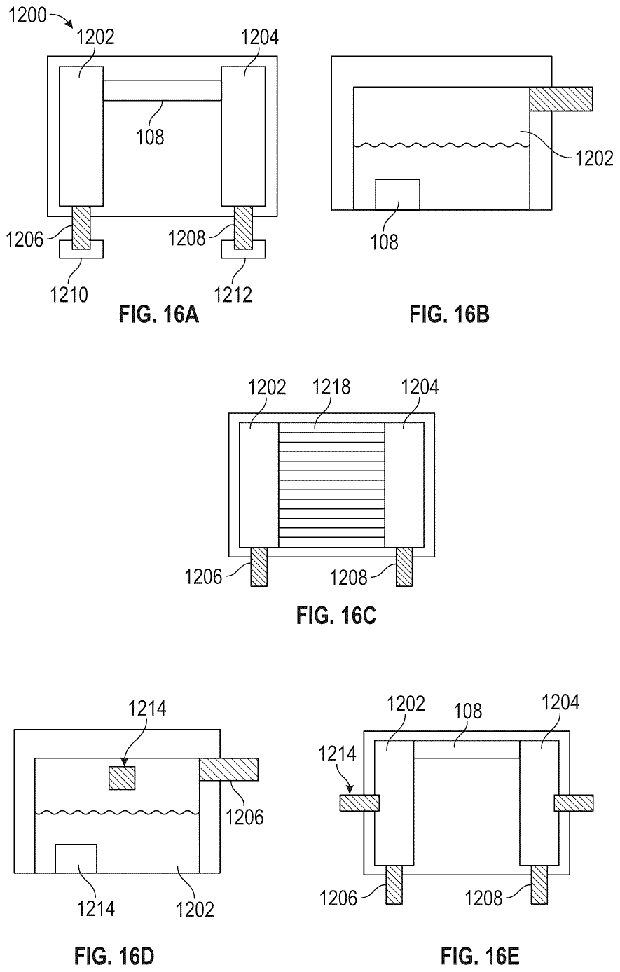

Further provided herein is a system for transfecting a plurality of cells with one or more exogenous molecules. The system as illustrated in FIG. 16A-E may be designed to transfect a plurality of cells with one or more exogenous molecules. The system may be operable to rapidly transfect the cells with the exogenous molecules much quicker than systems known in the art. Further, the system may be designed to transfect the cells with only one exogenous molecule multiple times, greatly increasing the successful transfection rate.

As illustrated in FIG. 16A, the system 1200 may be a microfluidic consumable which may include a first reservoir 1202, a second reservoir 1204, a first flow rate supply 1206, a second flow rate supply 1208, and a microfluidic transfection device 108.

As illustrated in FIG. 16A-16B, the microfluidic consumable may contain a first reservoir 1202. The first reservoir 1202 may be in fluid communication with a first flow rate supply 1206, a first valve 1210, and a microfluidic transfection device 108. The first reservoir 1202 may be operable to receive a first composition comprising a plurality of cells and a first exogenous molecule.

As illustrated in FIG. 16A-16B, first reservoir 1202 may include a first flow rate supply 1206. In an example, the first flow rate supply 1206 may be any device or instrument that provides a velocity to the cells in the system 1200. In one example, the first flow rate supply may be a first pressure supply to apply a pressure of about 5 psi to about 100 psi to the system to move the cells from the first reservoir through the microfluidic transfection device to the second reservoir.

As illustrated in FIG. 16A-16B, the system 1200 may include a first valve 1210. The first valve 1210 may have an open and a closed state. The first valve 1210 may be placed in an open state to allow the first flow rate supply 1206 to provide a flow rate to the first composition to move the first composition from the first reservoir through the microfluidic transfection device 108 to the second reservoir 1204. The first valve 1210 may be placed in a closed state when the first composition has passed through the microfluidic transfection device 108 to the second reservoir 1204.

As illustrated in FIG. 16A-16B, the system may include a microfluidic transfection device 108. The microfluidic transfection device 108 may comprise a first inlet, a transfection component, and a second inlet. The first inlet may be operable to receive the composition from the first reservoir 1202 in one direction and to receive transfected cells from the transfection component in the opposite direction. The second inlet may be operable to receive transfected cells from the transfection component in one direction and operable to receive a composition from the second reservoir 1204 in an opposite direction.

The transfection component may be operable to transfect the cells of the first composition with the first exogenous molecule to produce a first population of transfected cells.

The transfection component may be tuned or optimized for the transfection of cells based on the type of cells (e.g., size, stiffness, FACS characteristics, and/or physical characteristics) to be transfected.

As illustrated in FIG. 16A-16B, the system 1200 may include a second reservoir 1204. The second reservoir 1204 may be in fluid communication with a second flow rate supply 1208, a second valve 1212, and a microfluidic transfection device 108. The second reservoir 1204 may be operable to receive the first population of transfected cells.

As illustrated in FIG. 16A-16B, second reservoir 1204 may include a second flow rate supply 1208. In an example, the second flow rate supply 1208 may be any device or instrument that provides a velocity to the cells in the system. In one example, the second flow rate supply 1208 may be a second pressure supply to apply a pressure of about 5 psi to about 100 psi to the system 1200 to move the cells from the second reservoir 1204 through the microfluidic transfection device 108 to the first reservoir 1202.

As illustrated in FIG. 16A-16B, the system 1200 may include a second valve 1212. The second valve 1212 may have an open and a closed state. The second valve 1212 may be placed in an open state to allow the second flow rate supply 1208 to provide a flow rate to the first population of transfected cells to move the first population of transfected cells from the second reservoir 1204 through the microfluidic transfection device 108 to the first reservoir 1202. The second valve 1212 may be placed in a closed state when the first population of transfected cells has passed through the microfluidic transfection device 108 to the first reservoir 1202.

Passing the first population of transfected cells through the transfection component a second time increases the successful transfection percentage. In other examples, the first reservoir 1202 may have a first injector inlet in fluid communication with a first payload reservoir 1214, as illustrated, for example, in FIGS. 16D and 16E. The second reservoir 1204 may have a second injector inlet in fluid communication with a second payload reservoir 1216. The first payload reservoir 1214 may be operable to inject an exogenous molecule into the cells in the first reservoir 1202 and the cells may be transfected with the exogenous molecule by being passed through the microfluidic transfection device 108. The second payload reservoir 1216 may be operable to inject an exogenous molecule into the cells in the second reservoir 1204 and the cells may be transfected with the exogenous molecule by being passed through the microfluidic transfection device 108.

As illustrated in FIG. 16C, the system 1200 may be a high throughput microfluidic consumable. The high throughput microfluidic consumable may have multiple microfluidic transfection components 1218.

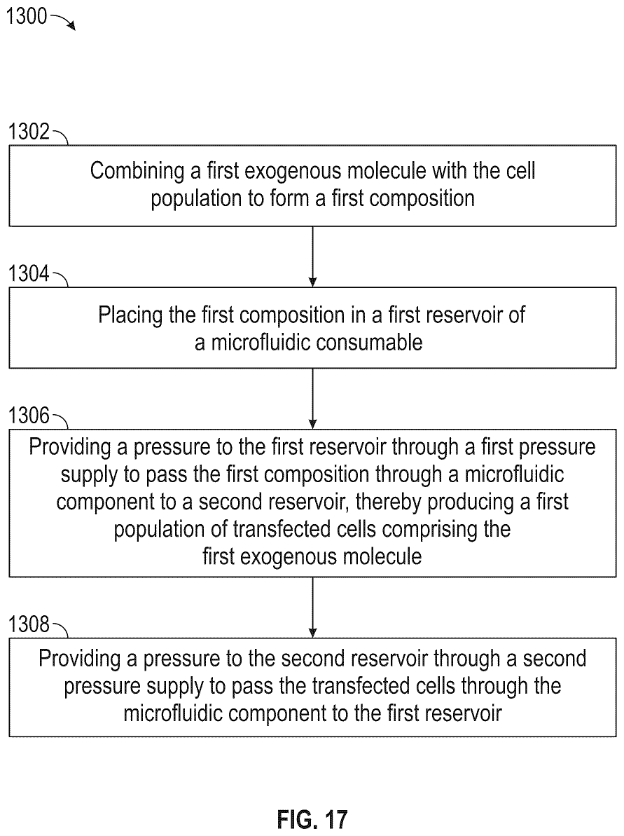

Also described herein is a method for transfecting a plurality of cells with at least one exogenous molecule. The method 1300 as illustrated in FIG. 17 may be designed to transfect a plurality of cells with one or more exogenous molecules. The method 1300 may rapidly transfect the cells with the exogenous molecules much quicker than systems known in the art. Further, the method may be designed to transfect the cells with only one exogenous molecule multiple times, greatly increasing the successful transfection rate.

As illustrated in FIG. 17, the method 1300 may begin at a first step 1302. At the first step 1302, the method 1300 may include combining a first exogenous molecule with a cell population to form a first composition.

As illustrated in FIG. 17, at a second step 1304, the method 1300 may include placing the first composition in the first reservoir of a microfluidic consumable.

A microfluidic transfection component in fluid communication with the first reservoir and a second reservoir may be tuned based on the size, stiffness, FACS characteristics, or other physical properties of the cells to optimize the transfection of the cells with the first exogenous molecule. The transfection component may be tuned by increasing or decreasing the gap size, or as otherwise discussed above.

As illustrated in FIG. 17, at a third step 1306, the method 1300 may include providing a pressure to the first reservoir through a first pressure supply to pass the first composition through a microfluidic component to a second reservoir producing a first population of transfected cells comprising the first exogenous molecule.

As illustrated in FIG. 17, at a fourth step 1308, the method 1300 may include providing a pressure to the second reservoir through a second pressure supply to pass the transfected cells through the microfluidic component to the first reservoir.

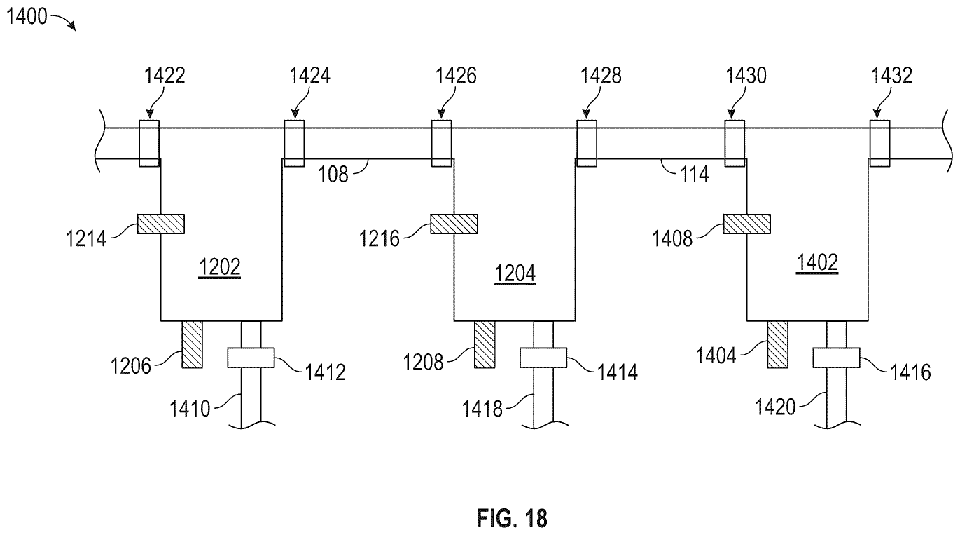

Also presented herein is a system for transfecting a plurality of cells with at least one exogenous molecule. The system 1400 of FIG. 18 may be configured to transfect a plurality of cells with a plurality of exogenous molecules sequentially. The system 1400 may be self-contained and capable of providing a sample after each transfection event. Further, the system 1400 may be operable to produce multiple transfection events much quicker than systems known in the art, while maintaining the same or better successful transfection rates and viability percentages.

As illustrated in FIG. 18, the system may have many of the components of the system of FIGS. 16A-E. As illustrated in FIG. 18, the system 1400 may include a first reservoir 1202, a second reservoir 1204, a third reservoir 1402, a first microfluidic transfection device 108, and a second microfluidic transfection device 114.

As illustrated in FIG. 18, the first reservoir may include a first cell inlet, a first cell inlet valve 1422, a first flow rate supply 1206, a first sample port 1410, a first payload reservoir 1214, a first sample port valve 1412, and an outlet (e.g., in fluid communication with the inlet valve 1424 of the first microfluidic transfection device 108). The first cell inlet may provide a plurality of cells to the first reservoir 1202. The first payload reservoir 1214 may provide a first exogenous molecule to the plurality of cells in the first reservoir 1202, thereby forming a first composition in the first reservoir 1202.

The first flow rate supply 1206 may be any device or instrument that provides a velocity to the cells in the system. In one example, the first flow rate supply 1206 may be a first pressure supply to apply a pressure of about 5 psi to about 100 psi to the system to move the cells from the first reservoir 1202 through the first microfluidic transfection device 108 to the second reservoir 1204.

As illustrated in FIG. 18, the first reservoir 1202 may have a first sample port 1410. In an example, the first sample port 1410 may be operable to remove a sample of the plurality of cells to test the cells. The sample port 1410 may be connected to a first cell testing device. In an example, the first cell testing device (e.g., analytical instrument) may be a flow cytometer, a cell counter, a next generation sequencing instrument, or other analytical instrument capable of analyzing the transfected cells configured to conduct an analysis of the sample. The first sample port 1410 may have a first sample port valve 1412 having an open state and a closed state. When a sample is to be removed the first sample port valve 1412 may be in an open state. A flow rate may be supplied by the first flow rate supply 1206 to the first reservoir 1202 to move a sample of the plurality of cells through the first sample port valve 1412 and into the first cell testing device. Once the sample has been removed, the sample port valve 1412 may be placed in the closed state.

As illustrated in FIG. 18, the system 1400 may have a first microfluidic transfection device 108. The first microfluidic transfection device 108 may comprise an inlet, an inlet valve 1424, a transfection component, an outlet, and an outlet valve 1426. The inlet valve may be configured to have an open state and a closed state. The inlet valve may be placed in an open state and a flow rate may be supplied to the first reservoir to move the first composition from the first reservoir to the first transfection component. Once the first composition has passed into the transfection component, the first inlet valve may be placed in a closed state.

The transfection component of the first microfluidic transfection device 108 may be operable to transfect the cells of the first composition with the first exogenous molecule to produce a first population of transfected cells. The transfection component of the first microfluidic transfection device 108 may be tuned or optimized for the transfection of cells based on the type of cells (e.g., size, stiffness, FACS characteristics, or physical characteristics) to be transfected.

As illustrated in FIG. 18, the outlet valve of the first microfluidic transfection device 108 may be placed in an open state to allow the first population of transfected cells to move through the first microfluidic transfection device 108 into the second reservoir 1204.

As illustrated in FIG. 18, the second reservoir 1204 may include a second cell inlet, a second flow rate supply 1208, a second sample port 1418, a second payload reservoir 1216, a second sample port valve 1414, and an outlet. The second cell inlet may allow the first population of transfected cells to enter into the second reservoir 1204. The second payload reservoir 1216 may provide a second exogenous molecule to the first population of transfected cells in the second reservoir 1204, thereby forming a second composition in the second reservoir 1204.

The second flow rate supply 1208 may be any device or instrument that provides a velocity to the cells in the system. In one example, the second flow rate supply 1208 may be a second pressure supply to apply a pressure of about 5 psi to about 100 psi to the system 1400 to move the cells from the second reservoir 1204 through the second microfluidic transfection device 114 to the third reservoir 1402.

The first population of transfected cells may be optionally held in the second reservoir 1204 for a sufficient period of time (e.g., a holding period) to allow the first population of transfected cells to recover from the first transfection event. In some examples, the holding period can allow the cells sufficient time and conditions to express one or more nucleotide sequences. In another example, the cells may be immediately injected with a second payload when they reach the second reservoir and have no holding period (e.g., no intervening culture step).

As illustrated in FIG. 18, the second reservoir 1204 may have a second sample port 1418. In an example, the second sample port 1418 may be operable to remove a sample of the first population of transfected cells to test the cells during the holding period. The sample port 1418 may be connected to a second cell testing device. In an example, the second cell testing device (e.g., analytical instrument) may be a flow cytometer, a cell counter, a next generation sequencing instrument, or other analytical instrument capable of analyzing the transfected cells. The cell testing device may be operable to analyze whether the cells were successfully transfected and the viability of the cells that were successfully transfected. The second sample port 1418 may have a second sample port valve 1414 having an open state and a closed state. When a sample is to be removed the second sample port valve 1414 may be in an open state. A flow rate may be supplied by the second flow rate supply 1208 to the second reservoir 1204 to move a sample of the first population of transfected cells through the second sample port valve 1414 and into the second cell testing device. Once the sample is removed, the second sample port valve 1414 may be placed in a closed state.

As illustrated in FIG. 18, the system 1400 may have a second microfluidic transfection device 114. The second microfluidic transfection device 114 may comprise a second inlet, a second inlet valve 1428, a transfection component, a second outlet, and a second outlet valve 1430. The second inlet valve 1428 may be configured to have an open state and a closed state. The second inlet valve 1428 may be placed in an open state and a flow rate may be supplied to the second reservoir 1204 to move the second composition from the second reservoir 1204 to the second microfluidic transfection device 114. Once the second composition has passed into the second microfluidic transfection component the second inlet valve 1428 may be placed in a closed state.

The transfection component of the second microfluidic transfection device may be operable to transfect the cells of the second composition with the second exogenous molecule to produce a second population of transfected cells. The transfection component of the second microfluidic transfection device may be tuned or optimized for the transfection of cells based on the type of cells (e.g., size, stiffness, FACS characteristics, or other physical characteristics) to be transfected.

As illustrated in FIG. 18, the second outlet valve 1430 of the second microfluidic transfection device 114 may be placed in an open state to allow the second population of transfected cells to move through the second microfluidic transfection device 114 into the third reservoir 1402.

As illustrated in FIG. 18, the third reservoir 1402 may include a third cell inlet, a third flow rate supply 1404, a third sample port 1420, a third payload reservoir 1408, a third sample port valve 1416, and an outlet. The third cell inlet may allow the second population of transfected cells to enter into the third reservoir 1402. The third payload reservoir 1408 may provide a third exogenous molecule to the second population of transfected cells in the third reservoir 1402, thereby forming a third composition in the third reservoir 1402.

The third flow rate supply 1404 may be any device or instrument that provides a velocity to the cells in the system. In one example, the third flow rate supply 1404 may be a third pressure supply to apply a pressure of about 5 psi to about 100 psi to the system 1400 to move the third composition from the third reservoir 1402 through the microfluidic transfection device to a fourth reservoir.

The second population of transfected cells may be optionally held in the third reservoir 1402 for a sufficient period of time (e.g., a holding period) to allow the second population of transfected cells to recover from the second transfection event. In another example, the cells may be immediately injected with a third payload when they reach the third reservoir and have no holding period (e.g., no intervening culture step). In some examples, the holding period can allow the cells sufficient time and conditions to express one or more nucleotide sequences.

As illustrated in FIG. 18, the third reservoir 1402 may have a third sample port 1420. In an example, the third sample port 1420 may be operable to remove a sample of the second population of transfected cells to test the cells during the holding period. The sample port 1420 may be connected to a third cell testing device. In an example, the third cell testing device (e.g., analytical instrument) may be a flow cytometer, a cell counter, a next generation sequencing instrument, or other analytical instrument capable of analyzing the transfected cells. The cell testing device may be operable to analyze whether the cells were successfully transfected and the viability of the cells that were successfully transfected. The third sample port 1420 may have a third sample port valve 1416 having an open state and a closed state. When a sample is to be removed the third sample port valve 1416 may be in an open state. A flow rate may be supplied by the third flow rate supply 1404 to the third reservoir 1402 to move a sample of the second population of transfected cells through the third sample port valve 1416 and into the third cell testing device. Once the sample is removed, the third sample port valve 1416 may be placed in a closed state.

The system may include N reservoirs and N microfluidic transfection devices to transfect the plurality of cells with N exogenous molecules in N transfection events. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be used in all or some of the N transfection events.

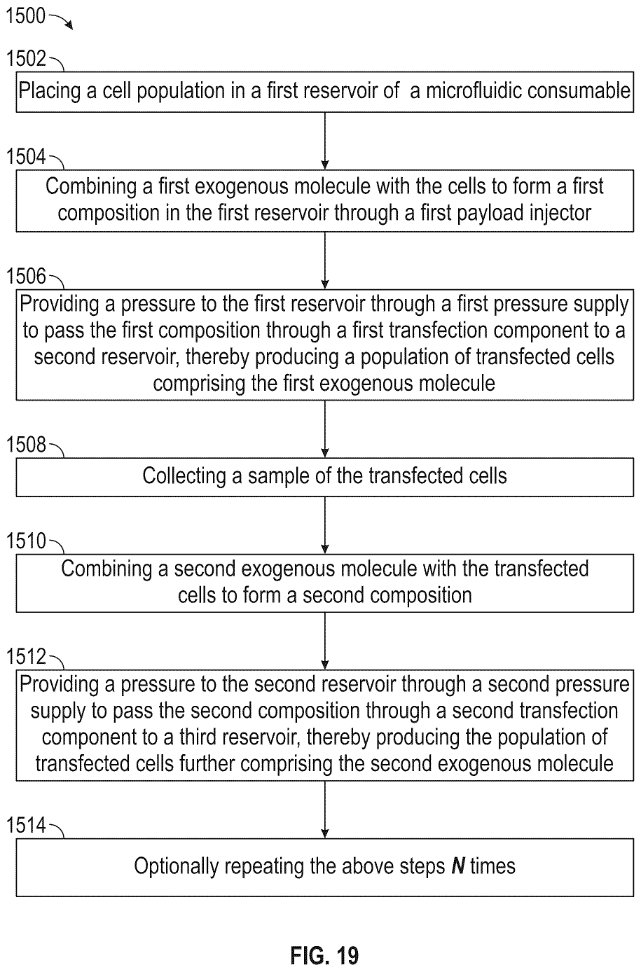

Further provided herein is a method for transfecting a plurality of cells with at least one exogenous molecule. The method 1500 of FIG. 19 may transfect a plurality of cells with a plurality of exogenous molecules sequentially. The method 1500 may be self-contained and capable of providing a sample after each transfection event. Further, the method 1500 may be operable to produce multiple transfection events much quicker than methods known in the art, while maintaining the same or better successful transfection rates and viability percentages.

As illustrated in FIG. 19, at a first step 1502, the method 1500 may include placing a cell population (e.g., plurality of cells) in a first reservoir of a microfluidic consumable.

As illustrated in FIG. 15, at a second step 1504, the method 1500 may include combining a first exogenous molecule with the cells to form a first cell composition in the first reservoir. The first exogenous molecule may be moved from the payload reservoir into the first reservoir by providing a flow rate (e.g., via a pressure).

As illustrated in FIG. 19, at a third step 1506, the method 1500 may include providing a pressure (e.g., through a first pressure supply) to the first reservoir to pass the first composition through a transfection component of a first microfluidic transfection device to a second reservoir. By passing the first composition through the transfection component, the plurality of cells may be transfected with the first exogenous molecule, producing a first population of transfected cells. In an example, the first microfluidic transfection device may be tuned or optimized for the first transfection event by increasing or decreasing the gap size or the other transfection parameters.

As illustrated in FIG. 19, at fourth step 1508, the method 1500 may include collecting a sample of transfected cells. In some examples, the sample may be collected by placing a second sample port valve in an open state and supplying a flow rate (e.g., via pressure from a second pressure supply) to the second reservoir to move a sample of cells through the sample port. In some examples, the sample may be removed while the cells are being held in the second reservoir for a holding period to allow sufficient recovery for the first population of transfected cells. In some examples, the hold period may be about 0.1 seconds to about 60 minutes.

As illustrated in FIG. 19, at a fifth step 1510, the method 1500 may include combining a second exogenous molecule with the first population of transfected cells to form a second composition. In some examples, the second payload reservoir may provide the second exogenous molecule to the second reservoir. In some examples, a flow rate (e.g., via a pressure) may be applied to the second payload reservoir to move the second exogenous molecule into the second reservoir.

As illustrated in FIG. 19, at a sixth step 1512, the method 1500 may include providing a pressure to the second reservoir through a second pressure supply to pass the second composition through a transfection component of a second microfluidic transfection device to a third reservoir. By passing the second composition through the transfection component, the first population of transfected cells may be transfected with the second exogenous molecule, producing a second population of transfected cells.

A sample may be removed from the third reservoir using a third pressure supply, placing a third sample port valve in an open state, and collecting the sample. In some examples, the sample may be removed while the second population of transfected cells is being held in a holding period to allow for sufficient recovery of the second population of transfected cells. In an example, the holding period may be about 0.1 seconds to about 60 minutes.

As illustrated in FIG. 19, at a seventh step 1514, the method 1500 may include repeating steps 1502-1508 N times. N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

The systems and methods provided herein may be combined to produce different results using components of the different systems. For example, the system of FIG. 5 and the system of FIG. 8 may be combined in a sequential processing scheme by using the system 100 for first and second transfection events and system 400 for further transfection events. In other examples, the different systems may be arranged sequentially to provide optimal cell transfection results.

The individual components of the systems and methods provided herein may be rearranged. For example, every system may have a cell sorter. In another example, some systems may not need a second payload reservoir, as the first payload reservoir can have a removable payload and continue to provide new payloads (e.g., exogenous molecules). The systems and methods provided herein may include some or other of the components discussed herein.

The systems and methods provided herein may not require an expansion step, meaning transfected cells may not require a long period of time between transfection events. Further, the methods provided herein may be fully automated. The systems and methods provided herein may be conducted in a self-contained environment. The cells may be transfected sequentially without removal from the device, cassette, consumable, or system.

In an aspect, the methods may further comprise counting the cells and spinning them down to the necessary cell density to transfect the cells using the sequential processing methods disclosed herein. The methods may further include resuspending the cells in native media or any similar buffer and/or media. The methods may further include mixing a payload (e.g., exogenous molecule) with the resuspended cells in a container (e.g., microfuge tube) before placing the cells in a reservoir or in another part of the system. In some examples, the methods may include spinning the cells down to a necessary cell density after the first transfection event. In some examples, the methods may include resuspending the cells in fresh native media or any similar buffer or media after spinning them down to a necessary density a second time. The methods may further include mixing a payload (e.g., exogenous molecule) with the resuspended cells (e.g., first population of transfected cells). The method may further include putting the cells back into an initial culture after transfecting them with the systems and methods disclosed herein. The expression of gene editing material may be observable within hours after transfecting a cell or cells using the systems and methods disclosed herein.

### (iv) Method of Sequential Editing

The disclosure further encompasses a method of sequential editing. The method comprises delivery of components for editing of one or more target sequences to a cell or a cell population. In certain aspects, the method comprises contacting a cell or a cell population, with one or more components of a genome editing system comprising guide RNA nucleotide sequence capable of hybridizing to one or more target sites, and the required nickase or a nucleotide sequence encoding the nickase. The cells are contacted sequentially, with the components to achieve desired editing of one or more target sites in the cell or cell population.

In one aspect, the method comprises contacting a cell or a cell population, with a first set of components of a genome editing system comprising guide RNA nucleotide sequence capable of hybridizing to a first target site; and at least with a second set of components of a genome editing system comprising a second guide RNA nucleotide sequence capable of hybridizing to a second target site. The cell or the cell population, in one aspect can be disposed in a microfluidic device disclosed herein.

In certain aspects, the method of sequential editing comprises contacting a cell or a cell population contained in microfluidic device disclosed herein, with a first set of components of a genome editing system comprising guide RNA nucleotide sequence capable of hybridizing to a first target site; and at least with a second set of components of a genome editing system comprising a second guide RNA nucleotide sequence capable of hybridizing to a second target site. The one or more components of the genome editing system can be delivered to cell or cell population using for example, a system described in FIG. 10.

The method can comprise an interval or a holding period between contacting with the first and the second set of genome editing components. The interval or holding period comprises holding the cell or population of cells in culture for a period of time. In some aspects, the holding period comprises about 1 hour to about 72 hours. In some aspects, the interval comprises about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, or more.

In some aspects, of the method, the cell for sequential editing is selected from an immune cell, a neural cell and a stem cell. In certain aspects, the cell is selected from a resting immune cell, optionally a resting T cell or a resting NK cell.

The cell or cell population disclosed herein, after the first event, for e.g., first genome editing, retain high transfection efficiency for further events or genome editing. Transfection efficiency can be determined as the percentage of cells that has the target gene edited or knocked out. In some aspects, the cell or cell population retain a transfection efficiency of about 20%, about 25%, about 30%, about 35%, about 40%, about 55%, about 60%, about 65%, about 70%, about 75%, or about 80%. In further aspects, the cell or cell population retain a transfection efficiency of about 20%, about 25%, about 30%, about 35%, about 40%, about 55%, about 60%, about 65%, about 70%, about 75%, or about 80% after being transfected with the first event. In an example, the cell or cells may retain a successful transfection percentage of about 20% to about 40%.

About 50%, about 55%, about 60%, about 65%, about 70%, about 75%, about 80%, about 85%, or about 90% of the cells may remain viable after being transfected using the first transfection device. In an example, about 50% to about 90% of the cells may remain viable after being transfected.

Further, the cell or cell population disclosed herein, retain high viability. In some aspects, about 50%, about 55%, about 60%, about 65%, about 70%, about 75%, about 80%, about 85%, or about 90% of the cells may remain viable after the first event for e.g., first genome editing. In certain aspects, about 50%, about 55%, about 60%, about 65%, about 70%, about 75%, about 80%, about 85%, or about 90% of the cells remain viable after the cells being transfected with first event. In an example, about 50% to about 90% of the cells may remain viable after being transfected with first event.

The expansion capacity of cell or cell population is further preserved after sequential editing. In some aspects, the cell or cell population retains 20%, about 25%, about 30%, about 35%, about 40%, about 55%, about 60%, about 65%, about 70%, about 75%, or about 80% expansion capacity.

Sequential editing as disclosed herein preserves stemness of the cell or cell population. In one aspect, sequential editing preserve population of cell that are stem-like (Tscm). Stem-like cells can be quantified by identifying cell subsets, for example, T cell subsets, using methods known in the art. In certain aspects, after priming preserves stem-like cells by about 20%, about 25%, about 30%, about 35%, about 40%, about 55%, about 60%, about 65%, about 70%, about 75%, or about 80%. In further aspects, the cell or cell population retain a stemness of about 20%, about 25%, about 30%, about 35%, about 40%, about 55%, about 60%, about 65%, about 70%, about 75%, or about 80% after priming, as compared to cells not contacted with the cytokine. In one aspect, cells have a preserved stemness with about 50% cells in the population being Tscm population.

The cell or cells may further be cryopreserved before or between one or more editing.

Cryopreservation can comprise any known method in the art. In one aspects, cell or cells are cooled at about 1° C./min during cryopreservation. Cryopreservation temperature is about −80° C. to about −180° C., for example about −125° C. to about −140° C. Cryopreserved cells can be transferred to liquid nitrogen prior to thawing for use. Cryopreservation can also be done using a controlled-rate freezer. In certain aspects, cryopreserved cells are thawed at a temperature of about 25° C. to about 40° C., preferably to a temperature of about 37° C. In further aspects, the cell or cells retain capacity to expand post-thaw. Capacity to expand can be determined by measuring the total number of live cells or percentage of live cells using method known in the art. In one example, automatic cell counter can be used to measure live or dead cell number. Post-thaw, about 50%, about 55%, about 60%, about 65%, about 70%, about 75%, about 80%, about 85%, or about 90% of the cells may remain viable. In an example, about 50% to about 90% of the cells may remain viable after thaw.

### (v) Priming Cells

In further aspects, the method comprises priming a cell or cell population. Priming the cell or cell population comprises contacting the cell or cell population with a cytokine. In one aspect, the cytokine can be added to the cell culture. Cytokine used for priming can be one or more of IL-2, IL-7, and IL-15. In one aspect, cytokine comprises a cocktail or mixture of cytokines IL-2, IL-7, and IL-15, and priming comprises adding the cytokine cocktail or mixture to culture medium.

In one aspect, the cell or cell population is contacted with a cytokine for about 1 hour to about 120 hours. The cell or cell population may be contacted with a cytokine for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, about 90 hours, about 91 hours, about 92 hours, about 93 hours, about 94 hours, about 95 hours, about 96 hours, about 97 hours, about 98 hours, about 99 hours, about 100 hours, about 101 hours, about 102 hours, about 103 hours, about 104 hours, about 105 hours, about 106 hours, about 107 hours, about 108 hours, about 109 hours, about 110 hours, about 111 hours, about 112 hours, about 113 hours, about 114 hours, about 115 hours, about 116 hours, about 117 hours, about 118 hours, about 119 hours, or about 120 hours. In exemplary aspect, cell or cell population is primed with a cytokine for up to 72 hours.

In certain aspects, the primed cell or cell population comprise an immune cell, a neural cell and a stem cell. In certain aspects, the cell is selected from a resting immune cell, optionally a resting T cell or a resting NK cell. In certain aspect, priming comprises contacting a resting T cell or resting T cell population with a cytokine.

Priming, as disclosed herein preserves stemness of the cell or cell population. In one aspect, priming with a cytokine can preserve population of cell that are stem-like (Tscm). Stem-like cells can be quantified by identifying cell subsets, for example, T cell subsets, using methods known in the art. In certain aspects, after priming preserves stem-like cells by about 20%, about 25%, about 30%, about 35%, about 40%, about 55%, about 60%, about 65%, about 70%, about 75%, or about 80%. In further aspects, the cell or cell population retain a stemness of about 20%, about 25%, about 30%, about 35%, about 40%, about 55%, about 60%, about 65%, about 70%, about 75%, or about 80% after priming, as compared to cells not contacted with the cytokine. In one aspect, cells have a preserved stemness with about 50% cells in the population being Tscm population.

Priming further retains transfection efficiency. Transfection efficiency can be determined as the percentage of cells that has the target gene edited or knocked out. In some aspects, the cell or cell population has a transfection efficiency of about 20%, about 25%, about 30%, about 35%, about 40%, about 55%, about 60%, about 65%, about 70%, about 75%, or about 80% after priming, as compared to cells not contacted with cytokine. In further aspects, the cell or cell population has a transfection efficiency of about 20%, about 25%, about 30%, about 35%, about 40%, about 55%, about 60%, about 65%, about 70%, about 75%, or about 80% using the transfection device disclosed herein. In an example, the cell or cells may have a successful transfection percentage of about 20% to about 40%. In certain aspects, cells have about 80% transfection efficiency.

In further aspects, about 50%, about 55%, about 60%, about 65%, about 70%, about 75%, about 80%, about 85%, or about 90% of the cells remain viable after priming, compared to cells not contacted with cytokine. In an example, about 50% to about 90% of the cells remain viable after priming for transfection using the transfection device disclosed herein. In one aspect, cells have about 70% viability.

The expansion capacity of cell or cell population is further preserved after priming. In some aspects, the cell or cell population retains 20%, about 25%, about 30%, about 35%, about 40%, about 55%, about 60%, about 65%, about 70%, about 75%, or about 80% expansion capacity.

### Examples

Example 1: The disclosed cell engineering method was tested using three sets of components to perform a non-viral knock-in on resting T-cells to produce CAR-T cells. FIGS. 1 and 2 illustrate the method conducted for this example. First, resting T-cells were prepared in a culture comprising IL-2, IL-7, and IL-15 to have a desired metabolic activity without reaching an activated state for two days, i.e., primed for two days, as illustrated in FIG. 1. The resting T-cells were then transfected with a first set of components. FIG. 3 illustrates a first set of components used in the example. The set of components (e.g., first plasmid) comprised gRNA, a U6 promoter, pegRNA, another U6 promoter, a CMV enhancer, Cas9, M-MLV, and BGH-polyA, as illustrated in FIG. 3. FIG. 3 illustrates the more detailed structural design of the first set of components. As illustrated in FIG. 3, the first set of components is a PE3b-AAVS1 vector.

The first transfection event was conducted using a microfluidic transfection device. The transfection event produced modified resting T-cells comprising an attP attachment site. After the first transfection event, the modified resting T-cells were held in culture (e.g., holding period) for one day to allow for sufficient recovery and expression of the attP attachment site.

After the one day holding period, the modified resting T-cells were transfected a second time with a second set of components and a third set of components producing a co-transfection event. As illustrated in FIG. 4, the second set of components comprised a serine integrase (PhiC31). As illustrated in FIG. 4, the third set of components comprised an insert plasmid. FIG. 4 illustrates the detailed structure of the serine integrase (PhiC31). FIG. 4 illustrates the detailed structure of the insert plasmid, attB insert-CAR.

The second transfection event was conducted using a second microfluidic transfection device. The serine integrase plasmid (PhiC31) allows attachment of the CAR insert between the attB attachment site and the attP docking site. The second transfection event produced CAR-T cells at a high viability rate and a high rate of successful transfection. The higher viability rate compared to other CAR-T cell manufacturing methods relates to the ability of the method to non-virally knock-in the CAR insert without activating the T-cells. The resting T-cells and resulting CAR-T cells maintain potency because they are transfected in their resting or naïve state. Many benefits were observed in the resulting CAR-T cells. For example, the resulting CAR-T cells had a high potency, and the transfection process was completed in under 3 days. The shortened time period for producing CAR-T cells compared to the time periods of methods known in the art may result in more effective CAR-T cell treatments for patients in need of CAR-T cells.

Example 2: Cell viability and transfection efficiency after sequential editing were examined in activated T cells.

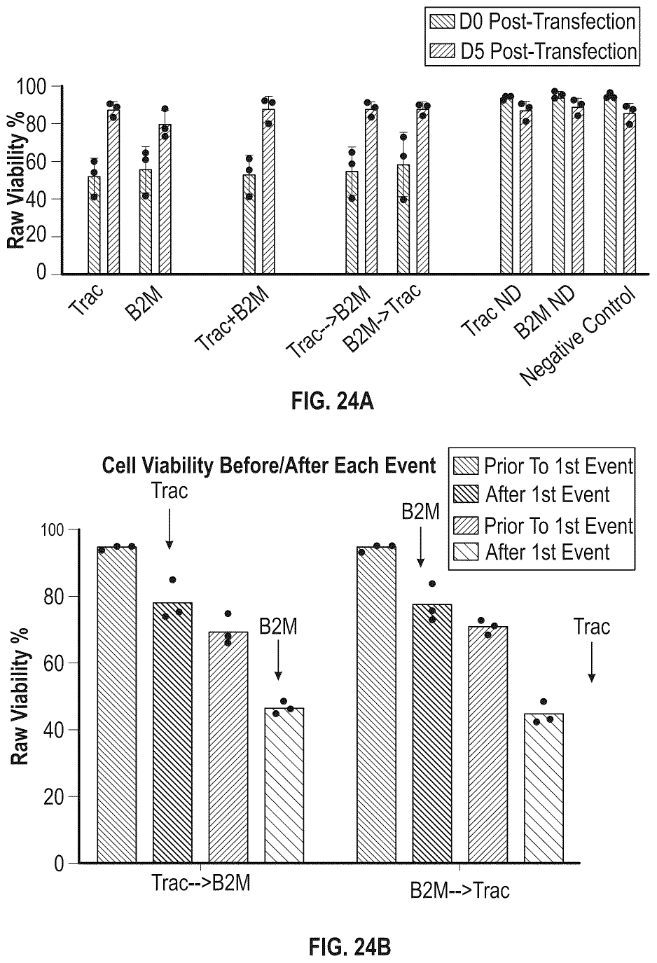

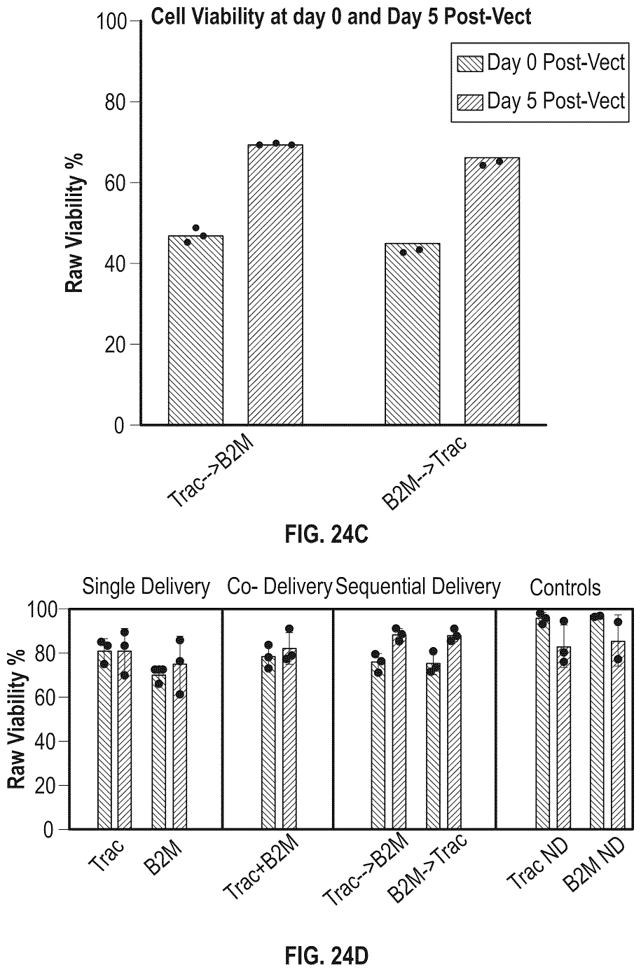

T-cells were transfected using the method described above. Editing was performed to knock out endogenous TRAC and B2M by sequential delivery of Cas9 RNP with a 1 hour interval (holding period), where cells are held in culture. Post-transfected T cells were subjected to automatic cell counter using NucleoCounter NC-3000 (chemometec Inc.) to measure live or dead cell number (AO/DAPI staining) and calculate the percentage of viability. T cell activation was performed for 48 hours. Cell viability was compared to single knock outs of TRAC or B2M, or simultaneous knockouts. Further a no device (“ND”) was used as a negative control with cell sample prepared in the same batch as experimental group but were not transfected. As shown in FIG. 24A, cell viability remained consistent between the edited T cells. Further, cell viability after each editing event was examined. Sequential editing was performed at 24 hour interval (holding period), and T cells were activated with CD3/CD28 stimulation for 24 hours. Cell viability of the edited T cells were comparable (FIG. 24B). Viability of cells were found to be enhanced at five days after transfection, as compared to day 0 (FIG. 24C). The viability of cells examined after single delivery of either TRAC or B2M editing Cas9 RNP, co-delivery of TRAC and B2M editing Cas9 RNP, sequential delivery of TRAC and B2M Cas9 RNP with a 24 hour interval (holding period), and with 6 hours of activation with CD3/CD28 stimulation were found to be similar between the different groups at 0 days and 5 days after transfection, as compared to cells from cell sample prepared in the same batch as experimental group but were not transfected (“ND”) (FIG. 24D).

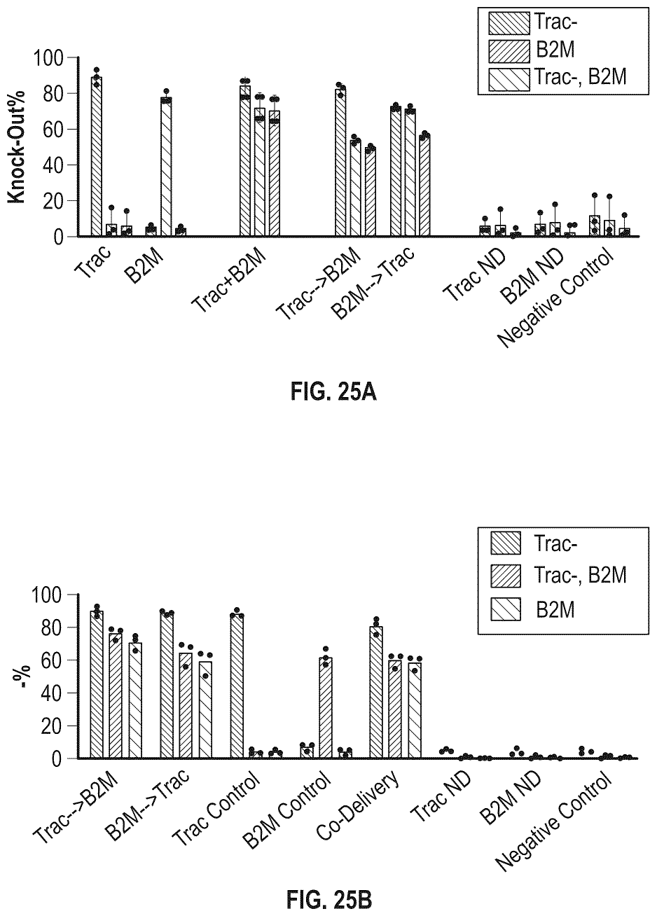

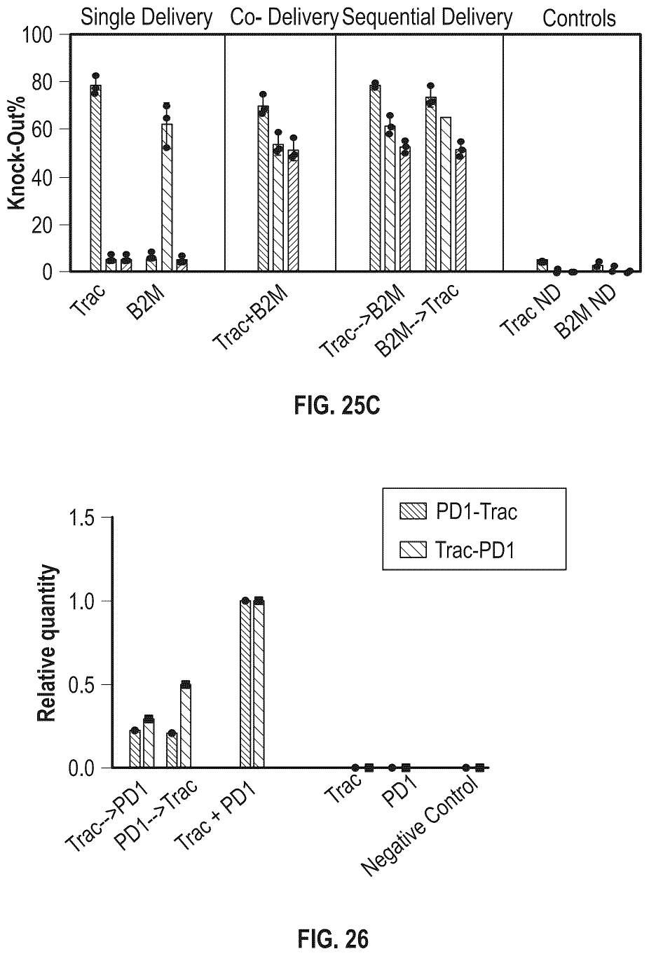

Transfection efficiency was additionally examined. Editing was performed to knock out TRAC and B2M by sequential delivery of Cas9 RNP at 1 hour interval (holding period). Post-transfected T-cells were cultured to the indicated time and subjected to flow cytometry (CytoFlex S, Beckman Coulter) analysis by staining the targeting marker on cell surface or detecting the reporter signal. T cell activation with CD3/CD28 stimulation was performed for 48 hours. Results were reported as a percentage to the control groups and data showed as average of biological replicates. Transfection efficiency was measured as the percentage of cells which didn't express the target protein. Percentage of cells with knock-out was found to be high with sequential editing indicating that the transfection efficiency remined high after sequential editing (FIG. 25A). Sequential editing of TRAC and B2M in T cells with a 24 hour interval, wherein the cells are held in culture (holding period) and a 24 hour T cell activation (1^{st} event) with CD3/CD28 stimulation exhibited high transfection efficiency similar to single editing of TRAC or B2M or co-delivery of TRAC and B2M (FIG. 25B). FIG. 25C further shows that cells with sequential delivery of TRAC and B2M editing Cas9 RNP with 24 hour interval (holding period) and 6 hour T cell activation with CD3/CD28 stimulation exhibited high percentage of knock-out as compared single delivery of TRAC or B2M Cas9-RNP and no device (ND) controls, and similar knock out percentages as co-delivery of TRAC and B2M editing Cas9 RNP.

Translocation events after sequential delivery of Cas9 RNP was examined. Frequency of translocation in T cells with sequential delivery of Cas9 RNP to knock out TRAC and PD1, or co-delivery of Cas9 RNP to knock out TRAC and PD1, was measured using chromosomal translocation assay by qPCR. T cells were activated with CD3/CD28 stimulation for 6 hours after first event. Sequential editing had a 24 hour interval (holding period) where cells are held in culture between delivery. Compared to co-delivery, sequential delivery reduced translocation event by about 75% (FIG. 26).

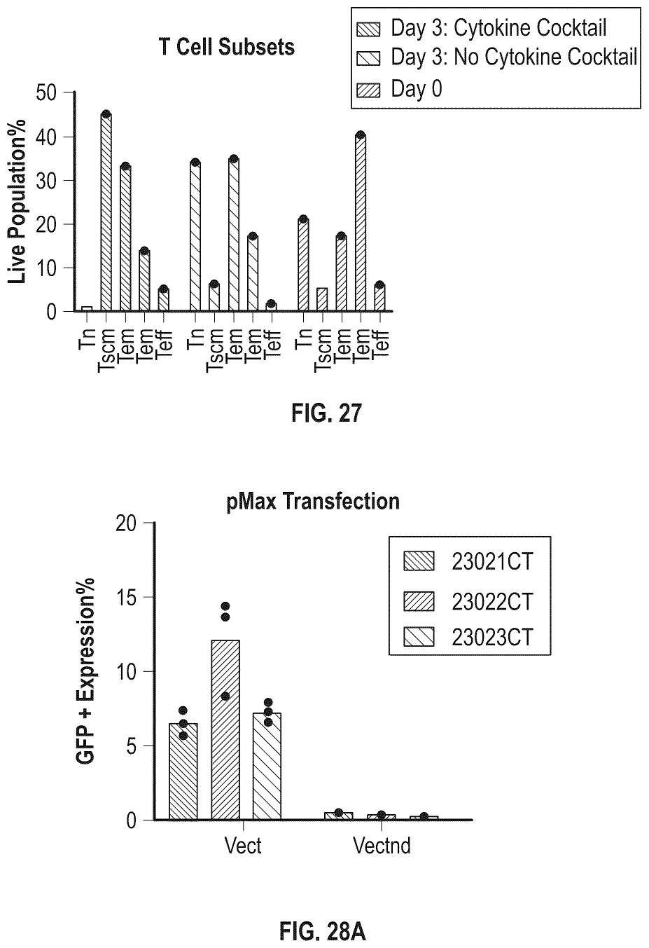

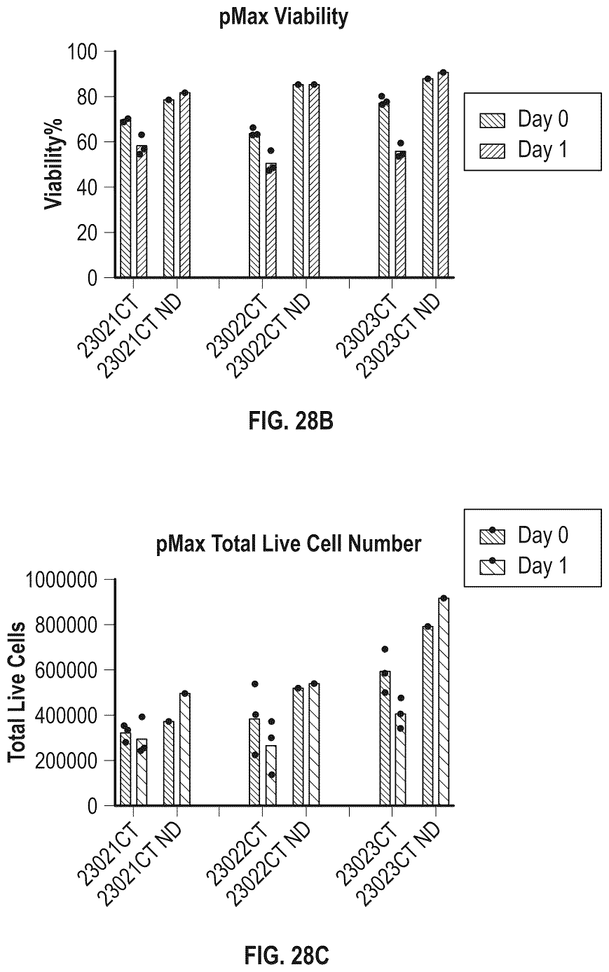

Example 3: A gene editing work flow was established to enable processing of resting T cells with high cell viability and transfection efficiency, while maintaining stemness with robust capacity to expand. Resting T cells were primed with a cytokine cocktail added to the cell culture for about 3 days. The cytokine cocktail included IL-2, IL-7, and IL-15. T cell subsets were quantified at Day 0 and at D3 after addition of cytokine cocktail. As shown in FIG. 27, priming with cytokine resulted in an improved population in stem cell like memory T cells (Tscm), compared to control culture without addition of cytokine cocktail. Plasmid delivery in cytokine primed resting T cells from three donors showed 5-15% transfection efficiency (FIG. 28A). Further, the cells with cytokine priming showed high viability (FIG. 28B) and high total live cell number (FIG. 28C).

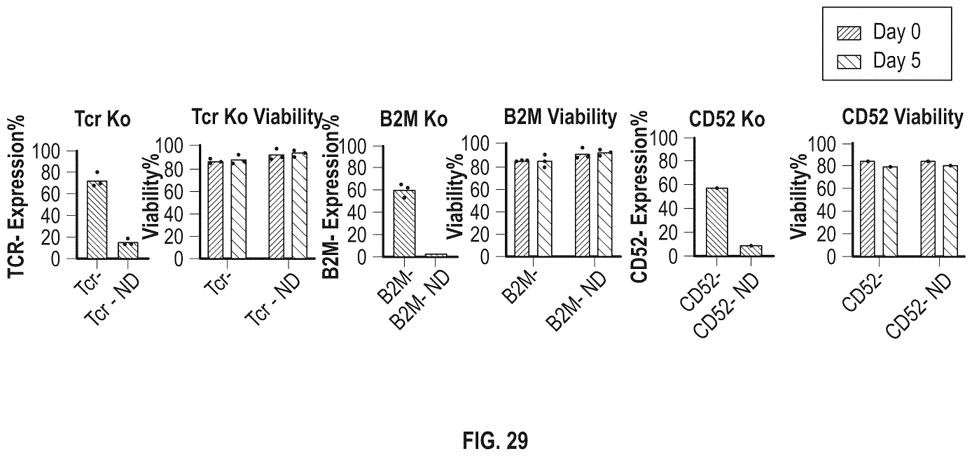

Transfection efficiency and viability of Cas9 RNP knockout in cytokine primed resting T cells were tested. The percentage of cells with knockout of TCR, B2M or CD52 were found to be between 60-80%, showing high transfection efficiency (FIG. 29). Additionally, the cytokine primed resting T cells with TCR, B2M or CD52 knock out exhibited high viabilities (FIG. 29).

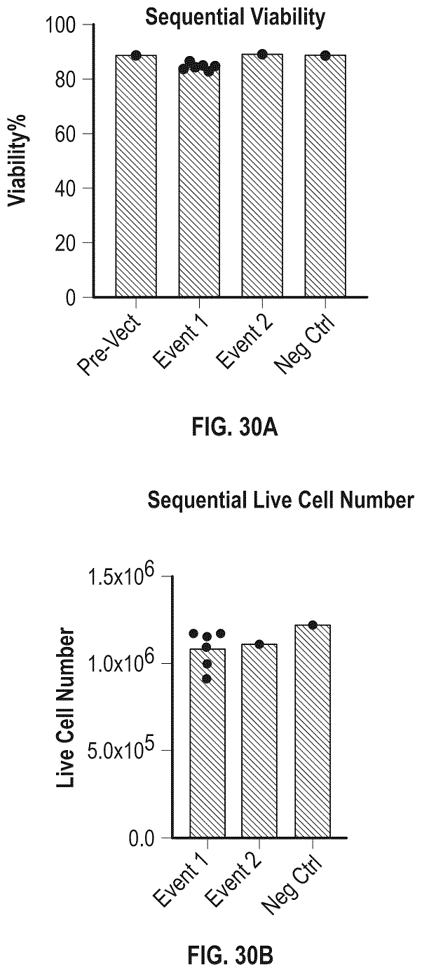

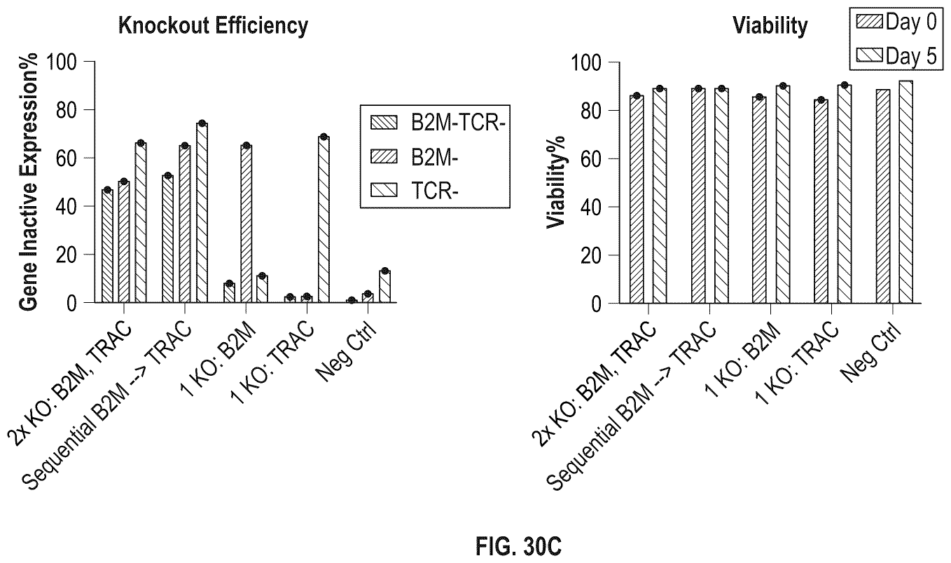

Multiplex or sequential knock outs were further tested on primed resting T cells. Primed resting T cells sequentially delivered with Cas9 RNP exhibited high cell viability (FIGS. 30A-30B). Further, primed resting T cells with either sequential or multiplex knock out of B2M and TRAC exhibited high knockout efficiency and viability (FIG. 30C).

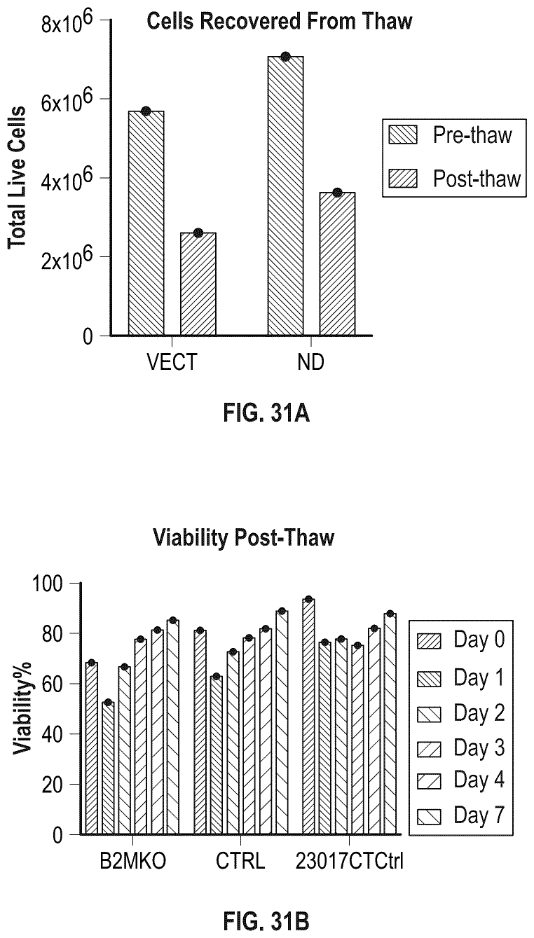

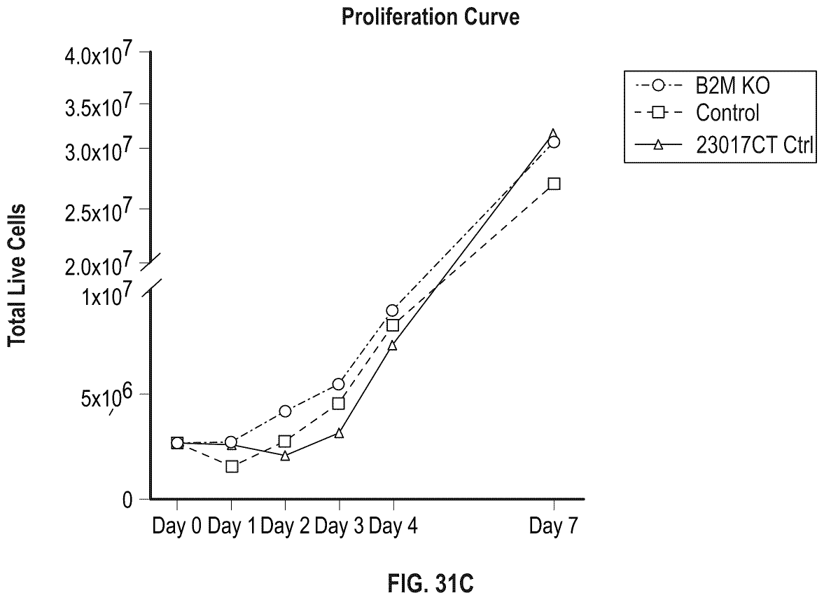

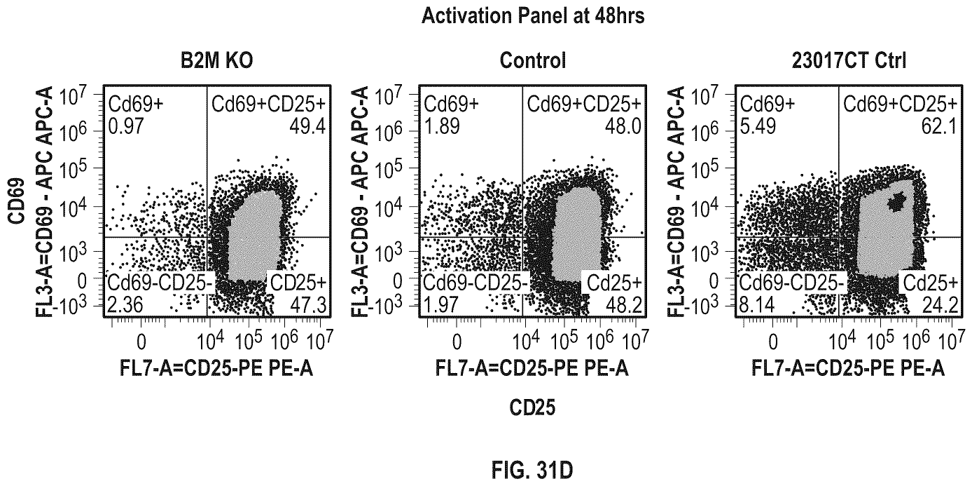

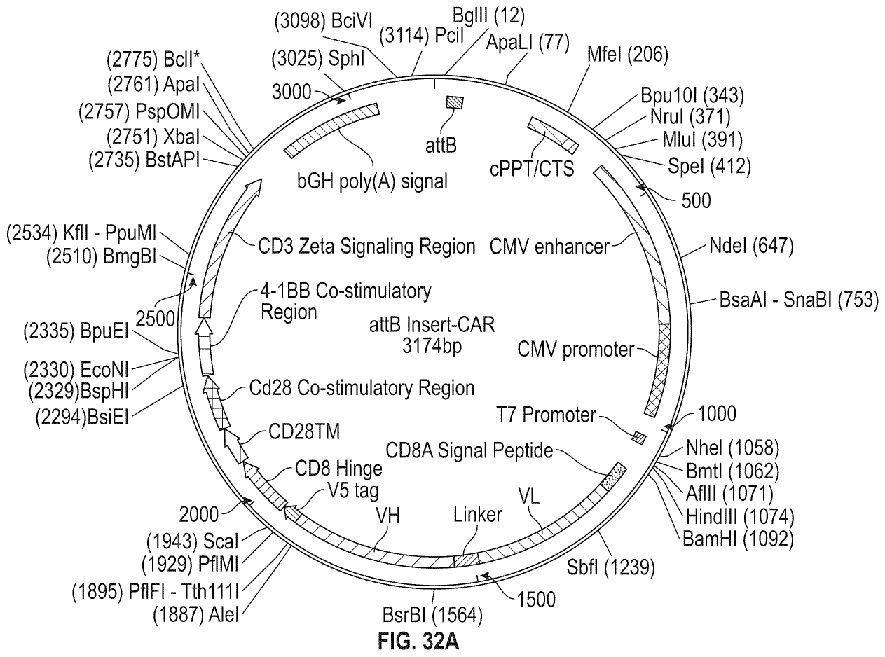

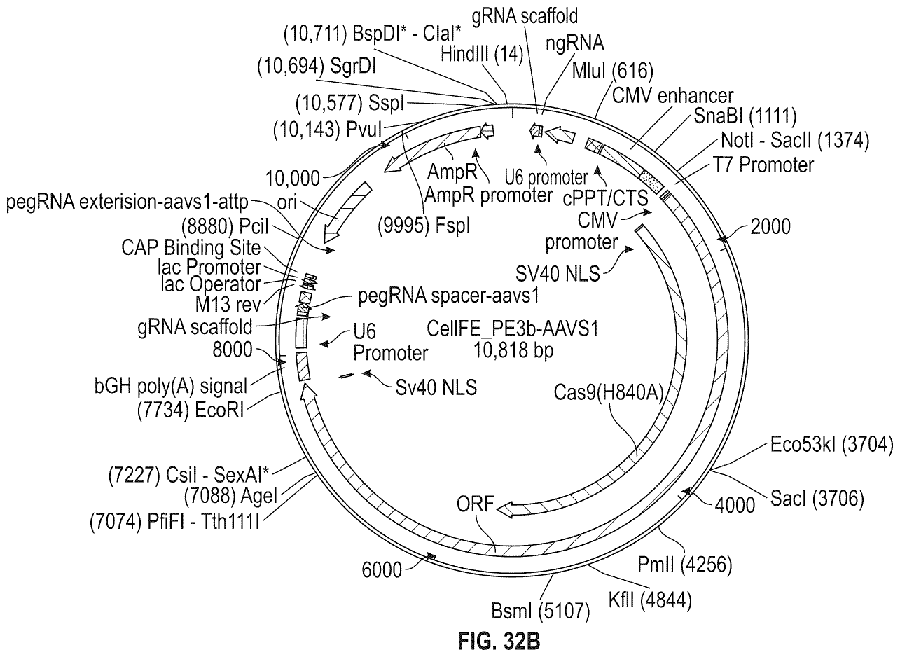

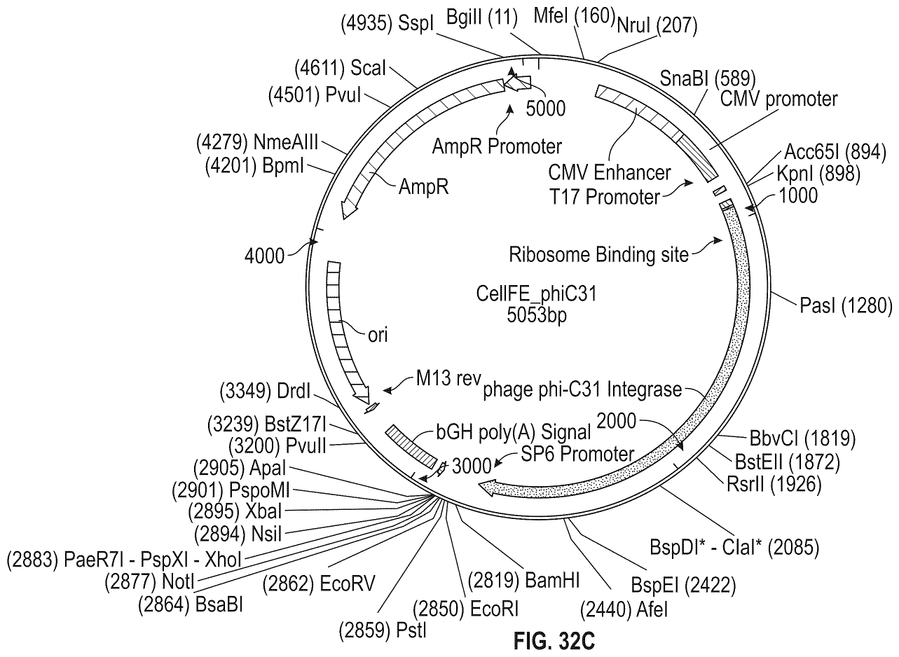

To test the effect of second round of freeze and thaw, thawed engineered resting T cells were cytokine-primed and transfected with Cas9 RNP to knock out B2M. The cells underwent a second round of freeze and thaw. Post-thaw cells were activated using CD3/CD28 co-stimulation. The activated post-thaw B2M knock out cells retained high viability (FIGS. 31A-31B). Post thaw B2M knock out cells further showed high capacity to expand comparable to non-processed cells (FIG. 31C). The post thaw B2M cells also exhibited similar activation with as compared to non-processed cells, at 48 hours (FIG. 31D).

The foregoing are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

TABLE 1
Sequence listing

| | | |
|---|---|---|
| SEQ | | |
| ID | | |
| NO | Type | Sequence |

NUCLEOTIDE SEQUENCES

| | | |
|---|---|---|
| 1 | CAR insert | gacggatcgggagatctcccgatgcgggtgccagggcgtgcccttgggctccccgggcgcgtactcctcccctat |
| | Plasmid | ggtgcactctcagtacaatctgctctgatgccgcatagttaagccagtatctgctccctgcttgtgtgttggaggtc |
| | whole | gctgagtagtgcgcgagcaaaatttaagctacaacaaggcaaggcttgaccgacaattgcatgttttaaaagaa |
| | sequence | aaggggggattggggggtacagtgcaggggaaagaatagtagacataatagcaacagacatacaaactaaag |
| | | aattacaaaaacaaattacaaaaattcaaaattttaagaatctgcttagggttaggcgttttgcgctgcttcgcga |
| | | tgtacgggccagatatacgcgttgacattgattattgactagttattaatagtaatcaattacggggtcattagttc |
| | | atagcccatatatggagttccgcgttacataacttacggtaaatggcccgcctggctgaccgcccaacgaccccc |
| | | gcccattgacgtcaataatgacgtatgttcccatagtaacgccaatagggactttccattgacgtcaatgggtgga |
| | | gtatttacggtaaactgcccacttggcagtacatcaagtgtatcatatgccaagtacgccccctattgacgtcaat |
| | | gacggtaaatggcccgcctggcattatgcccagtacatgaccttatgggactttcctacttggcagtacatctacgt |
| | | attagtcatcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttccaaaat |
| | | gtcgtaacaactccgccccattgacgcaaatgggcggtaggcgtgtacggtgggaggtctatataagcagagct |
| | | ctctggctaactagagaacccactgcttactggcttatcgaaattaatacgactcactataaggagacccaagct |
| | | ggctagcgtttaaacttaagcttggtaccgagctcggatccgccgccaccatggccttaccagtgaccgccttgct |
| | | cctgccgctggccttgctgctccacgccgccaggccggatatccagatgacccagacaacctccagcctgtccgc |
| | | ctccctgggcgatagagtgacaatctcctgcagggcctcccaggatatcagcaagtacctgaactggtaccagca |
| | | gaagcccgacggcacagtgaagctgctgatctaccacaccagcaggctgcactccggcgtgccctccaggttca |
| | | gcggctccggaagcggcaccgactacagcctgaccatcagcaatctggagcaggaggatatcgccacctactttt |
| | | gccagcagggcaataccctgccctacaccttcggcggcggcaccaagctggagatcaccggcagcacaagcgg |
| | | ctccggcaagcctggctccggcgaaggaagcacaaagggcgaggtgaagctgcaggagagcggccctggcctg |
| | | gtggctcctagccagtccctgtccgtgacctgtaccgtgtccggcgtgtccctgcctgactacggcgtgagctggat |
| | | caggcagccccccagaaagggcctggagtggctgggcgtgatctggggctccgagaccacatactacaattccg |
| | | ccctgaagagcaggctgaccatcatcaaggataatagcaagtcccaggtgtttctgaagatgaactccctgcag |
| | | accgacgacaccgccatctactactgcgccaagcactactactacggcggcagctacgccatggactactgggg |
| | | ccagggcaccagcgtgaccgtgtcctccggaaaacctatccctaatccactgcttggactcgatagtactaccact |
| | | accccagcaccgaggccacccaccccggctcctaccatcgcctcccagcctctgtccctgcgtccggaggcatgt |
| | | agacccgcagctggtggggccgtgcatacccggggtcttgacttcgcctgcgatttctgggtgctggtcgttgtgg |
| | | gcggcgtgctggcctgctacagcctgctggtgacagtggccttcatcatcttttgggtgaggagcaagcggagca |
| | | gactgctgcacagcgactacatgaacatgaccccccggaggcctggccccacccggaagcactaccagccctac |
| | | gcccctcccagggatttcgccgcctaccggagcaagcgcggtcggaagaagctgctgtacatctttaagcaaccc |
| | | ttcatgaggcctgtgcagactactcaagaggaggacggctgttcatgccggttcccagaggaggaggaaggcgg |
| | | ctgcgaactgcgcgtgaaattcagccgcagcgcagatgctccagcctacaagcaggggcagaaccagctctaca |
| | | acgaactcaatcttggtcggagagaggagtacgacgtgctggacaagcggagaggacgggacccagaaatgg |
| | | gcgggaagccgcgcagaaagaatccccaagagggcctgtacaacgagctccaaaaggataagatggcagaag |
| | | cctatagcgagattggtatgaaaggggaacgcagaagaggcaaaggccacgacggactgtaccagggactca |
| | | gcaccgccaccaaggacacctatgacgctcttcacatgcaggccctgccgcctcggtaatctagagggcccgttt |
| | | aaacccgctgatcagcctcgaaaaaaaaaaaaaaaaaaaaaaaaaaaagaagacggccctgtgccttctagt |
| | | tgccagccatctgttgtttgcccctcccccgtgccttccttgaccctggaaggtgccactcccactgtcctttcctaat |
| | | aaaatgaggaaattgcatcgcattgtctgagtaggtgtcattctattctggggggtggggggggcaggacagca |
| | | agggggaggattgggaagacaatagcaggcatgctggggatgcggtgggctctatggcttctgaggcggaaag |
| | | aaccagctggggctctagggggtatccccggggataacgcaggaaagaacatgtgagcaaaaggccagcaaa |
| | | aggccaggaaccgtaaaaaggccgcgttgctggcgtt |
| 2 | CD8a signal | atggccttaccagtgaccgccttgctcctgccgctggccttgctgctccacgccgccaggccg |
| | peptide | |
| 3 | VL | gatatccagatgacccagacaacctccagcctgtccgcctccctgggcgatagagtgacaatctcctgcagggcc |
| | | tcccaggatatcagcaagtacctgaactggtaccagcagaagcccgacggcacagtgaagctgctgatctacca |
| | | caccagcaggctgcactccggcgtgccctccaggttcagcggctccggaagcggcaccgactacagcctgacca |
| | | tcagcaatctggagcaggaggatatcgccacctacttttgccagcagggcaataccctgccctacaccttcggcg |
| | | gcggcaccaagctggagatcacc |
| 4 | Linker | ggcagcacaagcggctccggcaagcctggctccggcgaaggaagcacaaagggc |
| 5 | VH | gaggtgaagctgcaggagagcggccctggcctggtggctcctagccagtccctgtccgtgacctgtaccgtgtcc |
| | | ggcgtgtccctgcctgactacggcgtgagctggatcaggcagccccccagaaagggcctggagtggctgggcgt |
| | | gatctggggctccgagaccacatactacaattccgccctgaagagcaggctgaccatcatcaaggataatagca |
| | | agtcccaggtgtttctgaagatgaactccctgcagaccgacgacaccgccatctactactgcgccaagcactact |
| | | actacggcggcagctacgccatggactactggggccagggcaccagcgtgaccgtgtcctcc |
| 6 | CD8 hinge | accactaccccagcaccgaggccacccaccccggctcctaccatcgcctcccagcctctgtccctgcgtccggag |
| | | gcatgtagacccgcagctggtggggccgtgcatacccggggtcttgacttcgcctgcgat |
| 7 | CD8 TM | ttctgggtgctggtcgttgtgggcggcgtgctggcctgctacagcctgctggtgacagtggccttcatcatcttttgg |
| | | gtg |
| 8 | CD28 Co- | aggagcaagcggagcagactgctgcacagcgactacatgaacatgaccccccggaggcctggccccacccgga |
| | stimulatory | agcactaccagccctacgcccctcccagggatttcgccgcctaccggagc |
| | Region | |
| 9 | 4-IBB co- | aagcgcggtcggaagaagctgctgtacatctttaagcaacccttcatgaggcctgtgcagactactcaagagga |
| | stimulatory | ggacggctgttcatgccggttcccagaggaggaggaaggcggctgcgaactg |
| | region | |
| 10 | CDzeta | cgcgtgaaattcagccgcagcgcagatgctccagcctacaagcaggggcagaaccagctctacaacgaactca |
| | signaling | atcttggtcggagagaggagtacgacgtgctggacaagcggagaggacgggacccagaaatggggggaagc |
| | region | cgcgcagaaagaatccccaagagggcctgtacaacgagctccaaaaggataagatggcagaagcctatagcg |
| | | agattggtatgaaaggggaacgcagaagaggcaaaggccacgacggactgtaccagggactcagcaccgcca |
| | | ccaaggacacctatgacgctcttcacatgcaggccctgccgcctcgg |
| 11 | PE3b- | ccttattttaactaagcttaaaaaaatggggccgtgccccaactggggtaacctttgagttctctcagttggggga |
| | AAVS1 | ctagggacaggattggtgacagaaaagccccatcgcaccgactcggtgccactttttcaagttgataacggacta |
| | plasmid | gccttattttaacttgctatttctagctctaaaacagtggccccactgtggggtgcggtgatgctgaattacagaac |
| | whole | tcggtgtttcgtcctttccacaagatatataaagccaagaaatcgaaatactttcaagttacggtaagcatatgat |
| | sequence | agtccattttaaaacataattttaaaactgcaaactacccaagaaattattactttctacgtcacgtattttgtacta |
| | | atatctttgtgtttacagtcaaattaattctaattatctctctaacagccttgtatcgtatatgcaaatatgaaggaa |
| | | tcatgggaaataggccctcttcctgcccgaccttggtaccggatccagtcgacgccatttgtctgcagaattggcg |
| | | cacgcgctaaaaacggactagccttattttaacttgctatttctagctctaaaacaaaaaagcaccgactcggtgc |
| | | cactttttcaagttgataacggactagccttattttaacttgctatttctagctctaaaactgccccaactggggtaa |
| | | cctcggtgatggaggtgctgcggtgtttcgtcctttccacaagatatataaagccaagaaatcgaaatactttcaa |
| | | gttacggtaagcatatgatagtccattttaaaacataattttaaaactgcaaactacccaagaaattattactttct |
| | | acgtcacgtattttgtactaatatctttgtgtttacagtcaaattaattccaattatctctctaacagccttgtatcgt |
| | | atatgcaaatatgaaggaatcatgggaaataggccctcgcgagcaaaatttaagctacaacaaggcaaggctt |
| | | gaccgacaattgcatgaagaatctgcttagggttaggcgttttgcgctgcttcgcgatgtacgggccagatatacg |
| | | cgtttttaaaagaaaaggggggattggggggtacagtgcaggggaaagaatagtagacataatagcaacagac |
| | | atacaaactaaagaattacaaaaacaaattacaaaaattcaaaattttctagtccgtttttagggacattgattat |
| | | tgactagttattaatagtaatcaattacggggtcattagttcatagcccatatatggagttccgcgttacataactt |
| | | acggtaaatggcccgcctggctgaccgcccaacgacccccgcccattgacgtcaataatgacgtatgttcccata |
| | | gtaacgccaatagggactttccattgacgtcaatgggtggagtatttacggtaaactgcccacttggcagtacatc |
| | | aagtgtatcatatgccaagtacgccccctattgacgtcaatgacggtaaatggcccgcctggcattatgcccagta |
| | | catgaccttatgggactttcctacttggcagtacatctacgtattagtcatcgctattaccatggtgatgcggttttg |
| | | gcagtacatcaatgggcgtggatagcggtttgactcacggggatttccaagtctccaccccattgacgtcaatgg |
| | | gagtttgttttggcaccaaaatcaacgggactttccaaaatgtcgtaacaactccgccccattgacgcaaatggg |
| | | cggtaggcgtgtacggtgggaggtctatataagcagagctggtttagtgaaccgtcagatccgctagagatccgc |
| | | ggccgctaatacgactcactatagggagagccgccaccatgaaacggacagccgacggaagcgagttcgagtc |
| | | accaaagaagaagcggaaagtcgacaagaagtacagcatcggcctggacatcggcaccaactctgtgggctgg |
| | | gccgtgatcaccgacgagtacaaggtgcccagcaagaaattcaaggtgctgggcaacaccgaccggcacagca |
| | | tcaagaagaacctgatcggagccctgctgttcgacagcggcgaaacagccgaggccacccggctgaagagaac |
| | | cgccagaagaagatacaccagacggaagaaccggatctgctatctgcaagagatcttcagcaacgagatggcc |
| | | aaggtggacgacagcttcttccacagactggaagagtccttcctggtggaagaggataagaagcacgagcggc |
| | | accccatcttcggcaacatcgtggacgaggtggcctaccacgagaagtaccccaccatctaccacctgagaaag |
| | | aaactggtggacagcaccgacaaggccgacctgcggctgatctatctggccctggcccacatgatcaagttccgg |
| | | ggccacttcctgatcgagggcgacctgaaccccgacaacagcgacgtggacaagctgttcatccagctggtgca |
| | | gacctacaaccagctgttcgaggaaaaccccatcaacgccagcggcgtggacgccaaggccatcctgtctgcca |
| | | gactgagcaagagcagacggctggaaaatctgatcgcccagctgcccggcgagaagaagaatggcctgttcgg |
| | | aaacctgattgccctgagcctgggcctgacccccaacttcaagagcaacttcgacctggccgaggatgccaaact |
| | | gcagctgagcaaggacacctacgacgacgacctggacaacctgctggcccagatcggcgaccagtacgccgac |
| | | ctgtttctggccgccaagaacctgtccgacgccatcctgctgagcgacatcctgagagtgaacaccgagatcacc |
| | | aaggcccccctgagcgcctctatgatcaagagatacgacgagcaccaccaggacctgaccctgctgaaagctct |
| | | cgtgcggcagcagctgcctgagaagtacaaagagattttcttcgaccagagcaagaacggctacgccggctaca |
| | | ttgacggcggagccagccaggaagagttctacaagttcatcaagcccatcctggaaaagatggacggcaccga |
| | | ggaactgctcgtgaagctgaacagagaggacctgctgcggaagcagcggaccttcgacaacggcagcatcccc |
| | | caccagatccacctgggagagctgcacgccattctgcggcggcaggaagatttttacccattcctgaaggacaac |
| | | cgggaaaagatcgagaagatcctgaccttccgcatcccctactacgtgggccctctggccaggggaaacagcag |
| | | attcgcctggatgaccagaaagagcgaggaaaccatcaccccctggaacttcgaggaagtggtggacaagggc |
| | | gcttccgcccagagcttcatcgagcggatgaccaacttcgataagaacctgcccaacgagaaggtgctgcccaa |
| | | gcacagcctgctgtacgagtacttcaccgtgtataacgagctgaccaaagtgaaatacgtgaccgagggaatga |
| | | gaaagcccgccttcctgagcggcgagcagaaaaaggccatcgtggacctgctgttcaagaccaaccggaaagt |
| | | gaccgtgaagcagctgaaagaggactacttcaagaaaatcgagtgcttcgactccgtggaaatctccggcgtgg |
| | | aagatcggttcaacgcctccctgggcacataccacgatctgctgaaaattatcaaggacaaggacttcctggaca |
| | | atgaggaaaacgaggacattctggaagatatcgtgctgaccctgacactgtttgaggacagagagatgatcgag |
| | | gaacggctgaaaacctatgcccacctgttcgacgacaaagtgatgaagcagctgaagcggcggagatacaccg |
| | | gctggggcaggctgagccggaagctgatcaacggcatccgggacaagcagtccggcaagacaatcctggatttc |
| | | ctgaagtccgacggcttcgccaacagaaacttcatgcagctgatccacgacgacagcctgacctttaaagagga |
| | | catccagaaagcccaggtgtccggccagggcgatagcctgcacgagcacattgccaatctggccggcagccccg |
| | | ccattaagaagggcatcctgcagacagtgaaggtggtggacgagctcgtgaaagtgatgggccggcacaagcc |
| | | cgagaacatcgtgatcgaaatggccagagagaaccagaccacccagaagggacagaagaacagccgcgaga |
| | | gaatgaagcggatcgaagagggcatcaaagagctgggcagccagatcctgaaagaacaccccgtggaaaaca |
| | | cccagctgcagaacgagaagctgtacctgtactacctgcagaatgggcgggatatgtacgtggaccaggaactg |
| | | gacatcaaccggctgtccgactacgatgtggacgctatcgtgcctcagagctttctgaaggacgactccatcgac |
| | | aacaaggtgctgaccagaagcgacaagaaccggggcaagagcgacaacgtgccctccgaagaggtcgtgaag |
| | | aagatgaagaactactggcggcagctgctgaacgccaagctgattacccagagaaagttcgacaatctgacca |
| | | aggccgagagaggcggcctgagcgaactggataaggccggcttcatcaagagacagctggtggaaacccggc |
| | | agatcacaaagcacgtggcacagatcctggactcccggatgaacactaagtacgacgagaatgacaagctgat |
| | | ccgggaagtgaaagtgatcaccctgaagtccaagctggtgtccgatttccggaaggatttccagttttacaaagt |
| | | gcgcgagatcaacaactaccaccacgcccacgacgcctacctgaacgccgtcgtgggaaccgccctgatcaaaa |
| | | agtaccctaagctggaaagcgagttcgtgtacggcgactacaaggtgtacgacgtgcggaagatgatcgccaag |
| | | agcgagcaggaaatcggcaaggctaccgccaagtacttcttctacagcaacatcatgaactttttcaagaccgag |
| | | attaccctggccaacggcgagatccggaagcggcctctgatcgagacaaacggcgaaaccggggagatcgtgt |
| | | gggataagggccgggattttgccaccgtgcggaaagtgctgagcatgccccaagtgaatatcgtgaaaaagacc |
| | | gaggtgcagacaggcggcttcagcaaagagtctatcctgcccaagaggaacagcgataagctgatcgccagaa |
| | | agaaggactgggaccctaagaagtacggcggcttcgacagccccaccgtggcctattctgtgctggtggtggcca |
| | | aagtggaaaagggcaagtccaagaaactgaagagtgtgaaagagctgctggggatcaccatcatggaaagaa |
| | | gcagcttcgagaagaatcccatcgactttctggaagccaagggctacaaagaagtgaaaaaggacctgatcatc |
| | | aagctgcctaagtactccctgttcgagctggaaaacggccggaagagaatgctggcctctgccggcgaactgca |
| | | gaagggaaacgaactggccctgccctccaaatatgtgaacttcctgtacctggccagccactatgagaagctga |
| | | agggctcccccgaggataatgagcagaaacagctgtttgtggaacagcacaagcactacctggacgagatcatc |
| | | gagcagatcagcgagttctccaagagagtgatcctggccgacgctaatctggacaaagtgctgtccgcctacaa |
| | | caagcaccgggataagcccatcagagagcaggccgagaatatcatccacctgtttaccctgaccaatctgggag |
| | | cccctgccgccttcaagtactttgacaccaccatcgaccggaagaggtacaccagcaccaaagaggtgctggac |
| | | gccaccctgatccaccagagcatcaccggcctgtacgagacacggatcgacctgtctcagctgggaggtgactct |
| | | ggaggatctagcggaggatcctctggcagcgagacaccaggaacaagcgagtcagcaacaccagagagcagt |
| | | ggcggcagcagcggcggcagcagcaccctaaatatagaagatgagtatcggctacatgagacctcaaaagagc |
| | | cagatgtttctctagggtccacatggctgtctgattttcctcaggcctgggcggaaaccgggggcatgggactggc |
| | | agttcgccaagctcctctgatcatacctctgaaagcaacctctacccccgtgtccataaaacaataccccatgtca |
| | | caagaagccagactggggatcaagccccacatacagagactgttggaccagggaatactggtaccctgccagtc |
| | | cccctggaacacgcccctgctacccgttaagaaaccagggactaatgattataggcctgtccaggatctgagaga |
| | | agtcaacaagcgggtggaagacatccaccccaccgtgcccaacccttacaacctcttgagcgggctcccaccgtc |
| | | ccaccagtggtacactgtgcttgatttaaaggatgcctttttctgcctgagactccaccccaccagtcagcctctctt |
| | | cgcctttgagtggagagatccagagatgggaatctcaggacaattgacctggaccagactcccacagggtttca |
| | | aaaacagtcccaccctgtttaatgaggcactgcacagagacctagcagacttccggatccagcacccagacttg |
| | | atcctgctacagtacgtggatgacttactgctggccgccacttctgagctagactgccaacaaggtactcgggccc |
| | | tgttacaaaccctagggaacctcgggtatcgggcctcggccaagaaagcccaaatttgccagaaacaggtcaag |
| | | tatctggggtatcttctaaaagagggtcagagatggctgactgaggccagaaaagagactgtgatggggcagcc |
| | | tactccgaagacccctcgacaactaagggagttcctagggaaggcaggcttctgtcgcctcttcatccctgggttt |
| | | gcagaaatggcagcccccctgtaccctctcaccaaaccggggactctgtttaattggggcccagaccaacaaaa |
| | | ggcctatcaagaaatcaagcaagctcttctaactgccccagccctggggttgccagatttgactaagccctttgaa |
| | | ctctttgtcgacgagaagcagggctacgccaaaggtgtcctaacgcaaaaactgggaccttggcgtcggccggt |
| | | ggcctacctgtccaaaaagctagacccagtagcagctgggtggcccccttgcctacggatggtagcagccattgc |
| | | cgtactgacaaaggatgcaggcaagctaaccatgggacagccactagtcattctggccccccatgcagtagagg |
| | | cactagtcaaacaaccccccgaccgctggctttccaacgcccggatgactcactatcaggccttgcttttggacac |
| | | ggaccgggtccagttcggaccggtggtagccctgaacccggctacgctgctcccactgcctgaggaagggctgc |
| | | aacacaactgccttgatatcctggccgaagcccacggaacccgacccgacctaacggaccagccgctcccagac |
| | | gccgaccacacctggtacacggatggaagcagtctcttacaagagggacagcgtaaggcgggagctgcggtga |
| | | ccaccgagaccgaggtaatctgggctaaagccctgccagccgggacatccgctcagcgggctgaactgatagca |
| | | ctcacccaggccctaaagatggcagaaggtaagaagctaaatgtttatactgatagccgttatgcttttgctactg |
| | | cccatatccatggagaaatatacagaaggcgtgggtggctcacatcagaaggcaaagagatcaaaaataaaga |
| | | cgagatcttggccctactaaaagccctctttctgcccaaaagacttagcataatccattgtccaggacatcaaaag |
| | | ggacacagcgccgaggctagaggcaaccggatggctgaccaagcggcccgaaaggcagccatcacagagact |
| | | ccagacacctctaccctcctcatagaaaattcatcaccctctggcggctcaaaaagaaccgccgacggcagcga |
| | | attcgagcccaagaagaagaggaaagtctaatttaaacccgctgatcagcctcgactgtgccttctagttgccag |
| | | ccatctgttgtttgcccctcccccgtgccttccttgaccctggaaggtgccactcccactgtcctttcctaataaaat |
| | | gagaaaattgcatcgcattgtctgagtaggtgtcattctattctggggggtggggggggcaggacagcaagggg |
| | | gaggattgggaagacaatagcaggcatgctggggatgcggtgggctctatggcttctgaggcggaaagaacca |
| | | gctggggctcgataccgtcgacctctagctagagcttggcgtaatcatggtcatagctgtttcctgtgtgaaattgt |
| | | tatccgctcacaattccacacaacatacgagccggaagcataaagtgtaaagcctagggtgcctaatgagtgag |
| | | ctaactcacattaattgcgttgcgctcactgcccgctttccagtcgggaaacctgtcgtgccagctgcattaatgaa |
| | | tcggccaacgcgcggggagaggcggtttgcgtattgggcgctcttccgcttcctcgctcactgactcgctgcgctc |
| | | ggtcgttcggctgcggcgagcggtatcagctcactcaaaggcggtaatacggttatccacagaatcaggggata |
| | | acgcaggaaagaacatgtgagcaaaaggccagcaaaaggccaggaaccgtaaaaaggccgcgttgctggcgt |
| | | ttttccataggctccgcccccctgacgagcatcacaaaaatcgacgctcaagtcagaggtggcgaaacccgaca |
| | | ggactataaagataccaggcgtttccccctggaagctccctcgtgcgctctcctgttccgaccctgccgcttaccgg |
| | | atacctgtccgcctttctcccttcgggaagcgtggcgctttctcatagctcacgctgtaggtatctcagttcggtgta |
| | | ggtcgttcgctccaagctgggctgtgtgcacgaaccccccgttcagcccgaccgctgcgccttatccggtaactat |
| | | cgtcttgagtccaacccggtaagacacgacttatcgccactggcagcagccactggtaacaggattagcagagc |
| | | gaggtatgtaggcggtgctacagagttcttgaagtggtggcctaactacggctacactagaagaacagtatttgg |
| | | tatctgcgctctgctgaagccagttaccttcggaaaaagagttggtagctcttgatccggcaaacaaaccaccgct |
| | | ggtagcggtggtttttttgtttgcaagcagcagattacgcgcagaaaaaaaggatctcaagaagatcctttgatct |
| | | tttctacggggtctgacactcagtggaacgaaaactcacgttaagggattttggtcatgagattatcaaaaagga |
| | | tcttcacctagatccttttaaattaaaaatgaagttttaaatcaatctaaagtatatatgagtaaacttggtctgac |
| | | agttaccaatgcttaatcagtgaggcacctatctcagcgatctgtctatttcgttcatccatagttgcctgactcccc |
| | | gtcgtgtagataactacgatacgggagggcttaccatctggccccagtgctgcaatgataccgcgagacccacgc |
| | | tcaccggctccagatttatcagcaataaaccagccagccggaagggccgagcgcagaagtggtcctgcaacttt |
| | | atccgcctccatccagtctattaattgttgccgggaagctagagtaagtagttcgccagttaatagtttgcgcaac |
| | | gttgttgccattgctacaggcatcgtggtgtcacgctcgtcgtttggtatggcttcattcagctccggttcccaacga |
| | | tcaaggcgagttacatgatcccccatgttgtgcaaaaaagcggttagctccttcggtcctccgatcgttgtcagaa |
| | | gtaagttggccgcagtgttatcactcatggttatggcagcactgcataattctcttactgtcatgccatccgtaaga |
| | | tgcttttctgtgactggtgagtactcaaccaagtcattctgagaatagtgtatgcggcgaccgagttgctcttgccc |
| | | ggcgtcaatacgggataataccgcgccacatagcagaactttaaaagtgctcatcattggaaaacgttcttcggg |
| | | gcgaaaactctcaaggatcttaccgctgttgagatccagttcgatgtaacccactcgtgcacccaactgatcttca |
| | | gcatcttttactttcaccagcgtttctgggtgagcaaaaacaggaaggcaaaatgccgcaaaaaagggaataag |
| | | ggcgacacggaaatgttgaatactcatactcttcctttttcaatattattgaagcatttatcagggttattgtctcat |
| | | gagcggatacatatttgaatgtatttagaaaaataaacaaataggggttccgcgcacatttccccgaaaagtgcc |
| | | acctgacgtcgacggatcgggagatcgatctcccgatcccctagggtctactctcagtacaatctgctctgatgcc |
| | | gcatagttaagccagtatctgctccctgcttgtgtgttggaggtcgctgagtagtg |
| 12 | pegRNA | tggggccgtgccccaactggggtaacctttgagttctctcagttgggggactagggacaggattggtgacagaaa |
| | extension- | agccccatc |
| | aavs1-attp | |
| 13 | gRNA | gcaccgactcggtgccactttttcaagttgataacggactagccttattttaacttgctatttctagctctaaaac |
| | scaffold 1 | |
| 14 | pegRNA | agtggccccactgtggggtgcggtg |
| | spacer- | |
| | aavs1 | |
| 15 | gRNA | gcaccgactcggtgccactttttcaagttgataacggactagccttattttaacttgctatttctagctctaaaac |
| | scaffold 2 | |
| 16 | ngRNA | tgccccaactggggtaacctcggtg |
| 17 | cPPT/CTS | Ttttaaaagaaaaggggggattggggggtacagtgcaggggaaagaatagtagacataatagcaacagacat |
| | | acaaactaaagaattacaaaaacaaattacaaaaattcaaaatttt |
| 18 | Cas9(H840A) | gacaagaagtacagcatcggcctggacatcggcaccaactctgtgggctgggccgtgatcaccgacgagtacaa |
| | | ggtgcccagcaagaaattcaaggtgctgggcaacaccgaccggcacagcatcaagaagaacctgatcggagcc |
| | | ctgctgttcgacagcggcgaaacagccgaggccacccggctgaagagaaccgccagaagaagatacaccaga |
| | | cggaagaaccggatctgctatctgcaagagatcttcagcaacgagatggccaaggtggacgacagcttcttcca |
| | | cagactggaagagtccttcctggtggaagaggataagaagcacgagcggcaccccatcttcggcaacatcgtgg |
| | | acgaggtggcctaccacgagaagtaccccaccatctaccacctgagaaagaaactggtggacagcaccgacaa |
| | | ggccgacctgcggctgatctatctggccctggcccacatgatcaagttccggggccacttcctgatcgagggcga |
| | | cctgaaccccgacaacagcgacgtggacaagctgttcatccagctggtgcagacctacaaccagctgttcgagg |
| | | aaaaccccatcaacgccagcggcgtggacgccaaggccatcctgtctgccagactgagcaagagcagacggct |
| | | ggaaaatctgatcgcccagctgcccggcgagaagaagaatggcctgttcggaaacctgattgccctgagcctgg |
| | | gcctgacccccaacttcaagagcaacttcgacctggccgaggatgccaaactgcagctgagcaaggacacctac |
| | | gacgacgacctggacaacctgctggcccagatcggcgaccagtacgccgacctgtttctggccgccaagaacct |
| | | gtccgacgccatcctgctgagcgacatcctgagagtgaacaccgagatcaccaaggcccccctgagcgcctctat |
| | | gatcaagagatacgacgagcaccaccaggacctgaccctgctgaaagctctcgtgcggcagcagctgcctgaga |
| | | agtacaaagagattttcttcgaccagagcaagaacggctacgccggctacattgacggcggagccagccagga |
| | | agagttctacaagttcatcaagcccatcctggaaaagatggacggcaccgaggaactgctcgtgaagctgaaca |
| | | gagaggacctgctgcggaagcagcggaccttcgacaacggcagcatcccccaccagatccacctgggagagct |
| | | gcacgccattctgcggcggcaggaagatttttacccattcctgaaggacaaccgggaaaagatcgagaagatcc |
| | | tgaccttccgcatcccctactacgtgggccctctggccaggggaaacagcagattcgcctggatgaccagaaaga |
| | | gcgaggaaaccatcaccccctggaacttcgaggaagtggtggacaagggcgcttccgcccagagcttcatcgag |
| | | cggatgaccaacttcgataagaacctgcccaacgagaaggtgctgcccaagcacagcctgctgtacgagtactt |
| | | caccgtgtataacgagctgaccaaagtgaaatacgtgaccgagggaatgagaaagcccgccttcctgagcggc |
| | | gagcagaaaaaggccatcgtggacctgctgttcaagaccaaccggaaagtgaccgtgaagcagctgaaagag |
| | | gactacttcaagaaaatcgagtgcttcgactccgtggaaatctccggcgtggaagatcggttcaacgcctccctg |
| | | ggcacataccacgatctgctgaaaattatcaaggacaaggacttcctggacaatgaggaaaacgaggacattct |
| | | ggaagatatcgtgctgaccctgacactgtttgaggacagagagatgatcgaggaacggctgaaaacctatgccc |
| | | acctgttcgacgacaaagtgatgaagcagctgaagcggcggagatacaccggctggggcaggctgagccggaa |
| | | gctgatcaacggcatccgggacaagcagtccggcaagacaatcctggatttcctgaagtccgacggcttcgcca |
| | | acagaaacttcatgcagctgatccacgacgacagcctgacctttaaagaggacatccagaaagcccaggtgtcc |
| | | ggccagggcgatagcctgcacgagcacattgccaatctggccggcagccccgccattaagaagggcatcctgca |
| | | gacagtgaaggtggtggacgagctcgtgaaagtgatgggccggcacaagcccgagaacatcgtgatcgaaatg |
| | | gccagagagaaccagaccacccagaagggacagaagaacagccgcgagagaatgaagcggatcgaagagg |
| | | gcatcaaagagctgggcagccagatcctgaaagaacaccccgtggaaaacacccagctgcagaacgagaagc |
| | | tgtacctgtactacctgcagaatggggggatatgtacgtggaccaggaactggacatcaaccggctgtccgact |
| | | acgatgtggacgctatcgtgcctcagagctttctgaaggacgactccatcgacaacaaggtgctgaccagaagc |
| | | gacaagaaccggggcaagagcgacaacgtgccctccgaagaggtcgtgaagaagatgaagaactactggcgg |
| | | cagctgctgaacgccaagctgattacccagagaaagttcgacaatctgaccaaggccgagagaggcggcctga |
| | | gcgaactggataaggccggcttcatcaagagacagctggtggaaacccggcagatcacaaagcacgtggcaca |
| | | gatcctggactcccggatgaacactaagtacgacgagaatgacaagctgatccgggaagtgaaagtgatcacc |
| | | ctgaagtccaagctggtgtccgatttccggaaggatttccagttttacaaagtgcgcgagatcaacaactaccac |
| | | cacgcccacgacgcctacctgaacgccgtcgtgggaaccgccctgatcaaaaagtaccctaagctggaaagcga |
| | | gttcgtgtacggcgactacaaggtgtacgacgtgcggaagatgatcgccaagagcgagcaggaaatcggcaag |
| | | gctaccgccaagtacttcttctacagcaacatcatgaactttttcaagaccgagattaccctggccaacggcgag |
| | | atccggaagcggcctctgatcgagacaaacggcgaaaccggggagatcgtgtgggataagggccgggattttg |
| | | ccaccgtgcggaaagtgctgagcatgccccaagtgaatatcgtgaaaaagaccgaggtgcagacaggcggctt |
| | | cagcaaagagtctatcctgcccaagaggaacagcgataagctgatcgccagaaagaaggactgggaccctaag |
| | | aagtacggcggcttcgacagccccaccgtggcctattctgtgctggtggtggccaaagtggaaaagggcaagtc |
| | | caagaaactgaagagtgtgaaagagctgctggggatcaccatcatggaaagaagcagcttcgagaagaatccc |
| | | atcgactttctggaagccaagggctacaaagaagtgaaaaaggacctgatcatcaagctgcctaagtactccct |
| | | gttcgagctggaaaacggccggaagagaatgctggcctctgccggcgaactgcagaagggaaacgaactggcc |
| | | ctgccctccaaatatgtgaacttcctgtacctggccagccactatgagaagctgaagggctcccccgaggataat |
| | | gagcagaaacagctgtttgtggaacagcacaagcactacctggacgagatcatcgagcagatcagcgagttctc |
| | | caagagagtgatcctggccgacgctaatctggacaaagtgctgtccgcctacaacaagcaccgggataagccca |
| | | tcagagagcaggccgagaatatcatccacctgtttaccctgaccaatctgggagcccctgccgccttcaagtactt |
| | | tgacaccaccatcgaccggaagaggtacaccagcaccaaagaggtgctggacgccaccctgatccaccagagc |
| | | atcaccggcctgtacgagacacggatcgacctgtctcagctgggaggtgactctggaggatctagcggaggatc |
| | | ctct |
| 19 | phiC31 | acggatcgggagatctcccgatcccctatggtgcactctcagtacaatctgctctgatgccgcatagttaagccag |
| | plasmid | tatctgctccctgcttgtgtgttggaggtcgctgagtagtgcgcgagcaaaatttaagctacaacaaggcaaggct |
| | whole | tgaccgacaattgcatgaagaatctgcttagggttaggcgttttgcgctgcttcgcgatgtacgggccagatatac |
| | sequence | gcgttgacattgattattgactagttattaatagtaatcaattacggggtcattagttcatagcccatatatggagtt |
| | | ccgcgttacataacttacggtaaatggcccgcctggctgaccgcccaacgacccccgcccattgacgtcaataat |
| | | gacgtatgttcccatagtaacgccaatagggactttccattgacgtcaatgggtggagtatttacggtaaactgcc |
| | | cacttggcagtacatcaagtgtatcatatgccaagtacgccccctattgacgtcaatgacggtaaatggcccgcct |
| | | ggcattatgcccagtacatgaccttatgggactttcctacttggcagtacatctacgtattagtcatcgctattacca |
| | | tggtgatgcggttttggcagtacatcaatgggcgtggatagcggtttgactcacggggatttccaagtctccaccc |
| | | cattgacgtcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaactagagaacc |
| | | cactgcttactggcttatcgaaattaatacgactcactatagggagacccaagcttggtaccgagctcgctagaa |
| | | ataattttgtttaactttaagaaggagatatacatatggacacgtacgcgggtgcttacgaccgtcagtcgcgcga |
| | | gcgcgaaaattcgagcgcagcaagcccagcgacacagcgtagcgccaacgaagacaaggcggccgaccttca |
| | | gcgcgaagtcgagcgcgacgggggccggttcaggttcgtcgggcatttcagcgaagcgccgggcacgtcggcgt |
| | | tcgggacggcggagcgcccggagttcgaacgcatcctgaacgaatgccgcgccgggcggctcaacatgatcatt |
| | | gtctatgacgtgtcgcgcttctcgcgcctgaaggtcatggacgcgattccgattgtctcggaattgctcgccctggg |
| | | cgtgacgattgtttccactcaggaaggcgtcttccggcagggaaacgtcatggacctgattcacctgattatgcgg |
| | | ctcgacgcgtcgcacaaagaatcttcgctgaagtcggcgaagattctcgacacgaagaaccttcagcgcgaatt |
| | | gggcgggtacgtcggcgggaaggcgccttacggcttcgagcttgtttcggagacgaaggagatcacgcgcaacg |
| | | gccgaatggtcaatgtcgtcatcaacaagcttgcgcactcgaccactccccttaccggacccttcgagttcgagcc |
| | | cgacgtaatccggtggtggtggcgtgagatcaagacgcacaaacaccttcccttcaagccgggcagtcaagccg |
| | | ccattcacccgggcagcatcacggggctttgtaagcgcatggacgctgacgccgtgccgacccggggcgagacg |
| | | attgggaagaagaccgcttcaagcgcctgggacccggcaaccgttatgcgaatccttcgggacccgcgtattgcg |
| | | ggcttcgccgctgaggtgatctacaagaagaagccggacggcacgccgaccacgaagattgagggttaccgcat |
| | | tcagcgcgacccgatcacgctccggccggtcgagcttgattgcggaccgatcatcgagcccgctgagtggtatga |
| | | gcttcaggcgtggttggacggcagggggcgcggcaaggggctttcccgggggcaagccattctgtccgccatgg |
| | | acaagctgtactgcgagtgtggcgccgtcatgacttcgaagcgcggggaagaatcgatcaaggactcttaccgct |
| | | gccgtcgccggaaggtggtcgacccgtccgcacctgggcagcacgaaggcacgtgcaacgtcagcatggcggc |
| | | actcgacaagttcgttgcggaacgcatcttcaacaagatcaggcacgccgaaggcgacgaagagacgttggcg |
| | | cttctgtgggaagccgcccgacgcttcggcaagctcactgaggcgcctgagaagagcggcgaacgggcgaacct |
| | | tgttgcggagcgcgccgacgccctgaacgcccttgaagagctgtacgaagaccgcgcggcaggcgcgtacgac |
| | | ggacccgttggcaggaagcacttccggaagcaacaggcagcgctgacgctccggcagcaaggggcggaagag |
| | | cggcttgccgaacttgaagccgccgaagccccgaagcttccccttgaccaatggttccccgaagacgccgacgct |
| | | gacccgaccggccctaagtcgtggtgggggcgcgcgtcagtagacgacaagcgcgtgttcgtcgggctcttcgta |
| | | gacaagatcgttgtcacgaagtcgactacgggcagggggcagggaacgcccatcgagaagcgcgcttcgatca |
| | | cgtgggcgaagccgccgaccgacgacgacgaagacgacgcccaggacggcacggaagacgtagcggcgtagc |
| | | gagacacccggatcgcgccaaaaaaaaaaaaaaaaaaaaaaaaaaaaaaaccggatccactagtaacggcc |
| | | gccagtgtgctggaattctgcagatatccatcacactggcggccgctcgagcatgcatctagagggccctattcta |
| | | tagtgtcacctaaatgctagagctcgctgatcagcctcgactgtgccttctagttgccagccatctgttgtttgcccc |
| | | tcccccgtgccttccttgaccctggaaggtgccactcccactgtcctttcctaataaaatgaggaaattgcatcgca |
| | | ttgtctgagtaggtgtcattctattctggggggggggtggggcaggacagcaagggggaggattgggaagaca |
| | | atagcaggcatgctggggatgcggtgggctctatggcttctgaggcggaaagaaccagctggggctctaggggg |
| | | tatcccctgtggaattgtctgtataccgtcgacctctagctagagcttggcgtaatcatggtcatagctgtttcctgt |
| | | gggcgtttttccataggctccgcccccctgacgagcatcacaaaaatcgacgctcaagtcagaggtggcgaaac |
| | | ccgacaggactataaagataccaggcgtttccccctggaagctccctcgtgcgctctcctgttccgaccctgccgc |
| | | ttaccggatacctgtccgcctttctcccttcgggaagcgtggcgctttctcatagctcacgctgtaggtatctcagtt |
| | | cggtgtaggtcgttcgctccaagctgggctgtgtgcacgaaccccccgttcagcccgaccgctgcgccttatccgg |
| | | taactatcgtcttgagtccaacccggtaagacacgacttatcgccactggcagcagccactggtaacaggattag |
| | | cagagcgaggtatgtaggcggtgctacagagttcttgaagtggtggcctaactacggctacactagaagaacag |
| | | tatttggtatctgcgctctgctgaagccagttaccttcggaaaaagagttggtagctcttgatccggcaaacaaac |
| | | caccgctggtagcggtttttttgtttgcaagcagcagattacgcgcagaaaaaaaggatctcaagaagatcctttg |
| | | atcttttctacggggtctgacgctcagtggaacgaaaactcacgttaagggattttggtcatgagattatcaaaaa |
| | | ggatcttcacctagatccttttaaattaaaaatgaagttttaaatcaatctaaagtatatatgagtaaacttggtct |
| | | gacagttaccaatgcttaatcagtgaggcacctatctcagcgatctgtctatttcgttcatccatagttgcctgactc |
| | | cccgtcgtgtagataactacgatacgggagggcttaccatctggccccagtgctgcaatgataccgcgagaccca |
| | | cgctcaccggctccagatttatcagcaataaaccagccagccggaagggccgagcgcagaagtggtcctgcaac |
| | | tttatccgcctccatccagtctattaattgttgccgggaagctagagtaagtagttcgccagttaatagtttgcgca |
| | | acgttgttgccattgctacaggcatcgtggtgtcacgctcgtcgtttggtatggcttcattcagctccggttcccaac |
| | | gatcaaggcgagttacatgatcccccatgttgtgcaaaaaagcggttagctccttcggtcctccgatcgttgtcag |
| | | aagtaagttggccgcagtgttatcactcatggttatggcagcactgcataattctcttactgtcatgccatccgtaa |
| | | gatgcttttctgtgactggtgagtactcaaccaagtcattctgagaatagtgtatgcggcgaccgagttgctcttgc |
| | | ccggcgtcaatacgggataataccgcgccacatagcagaactttaaaagtgctcatcattggaaaacgttcttcg |
| | | gggcgaaaactctcaaggatcttaccgctgttgagatccagttcgatgtaacccactcgtgcacccaactgatctt |
| | | cagcatcttttactttcaccagcgtttctgggtgagcaaaaacaggaaggcaaaatgccgcaaaaaagggaata |
| | | agggcgacacggaaatgttgaatactcatactcttcctttttcaatattattgaagcatttatcagggttattgtctc |
| | | atgagcggatacatatttgaatgtatttagaaaaataaacaaataggggttccgcgcacatttccccgaaaagtg |
| | | ccacctgacgt |
| 20 | phage phi- | atggacacgtacgcgggtgcttacgaccgtcagtcgcgcgagcgcgaaaattcgagcgcagcaagcccagcga |
| | C31 | cacagcgtagcgccaacgaagacaaggcggccgaccttcagcgcgaagtcgagcgcgacgggggccggttca |
| | integrase | ggttcgtcgggcatttcagcgaagcgccgggcacgtcggcgttcgggacggcggagcgcccggagttcgaacgc |
| | | atcctgaacgaatgccgcgccgggcggctcaacatgatcattgtctatgacgtgtcgcgcttctcgcgcctgaagg |
| | | tcatggacgcgattccgattgtctcggaattgctcgccctgggcgtgacgattgtttccactcaggaaggcgtcttc |
| | | cggcagggaaacgtcatggacctgattcacctgattatgcggctcgacgcgtcgcacaaagaatcttcgctgaag |
| | | tcggcgaagattctcgacacgaagaaccttcagcgcgaattggggggtacgtcggcgggaaggcgccttacgg |
| | | cttcgagcttgtttcggagacgaaggagatcacgcgcaacggccgaatggtcaatgtcgtcatcaacaagcttgc |
| | | gcactcgaccactccccttaccggacccttcgagttcgagcccgacgtaatccggtggtggtggcgtgagatcaa |
| | | gacgcacaaacaccttcccttcaagccgggcagtcaagccgccattcacccgggcagcatcacggggctttgtaa |
| | | gcgcatggacgctgacgccgtgccgacccggggcgagacgattgggaagaagaccgcttcaagcgcctgggac |
| | | ccggcaaccgttatgcgaatccttcgggacccgcgtattgcgggcttcgccgctgaggtgatctacaagaagaag |
| | | ccggacggcacgccgaccacgaagattgagggttaccgcattcagcgcgacccgatcacgctccggccggtcga |
| | | gcttgattgcggaccgatcatcgagcccgctgagtggtatgagcttcaggcgtggttggacggcagggggcgcg |
| | | gcaaggggctttcccgggggcaagccattctgtccgccatggacaagctgtactgcgagtgtggcgccgtcatga |
| | | cttcgaagcgcggggaagaatcgatcaaggactcttaccgctgccgtcgccggaaggtggtcgacccgtccgca |
| | | cctgggcagcacgaaggcacgtgcaacgtcagcatggcggcactcgacaagttcgttgcggaacgcatcttcaa |
| | | caagatcaggcacgccgaaggcgacgaagagacgttggcgcttctgtgggaagccgcccgacgcttcggcaag |
| | | ctcactgaggcgcctgagaagagcggcgaacgggcgaaccttgttgcggagcgcgccgacgccctgaacgccct |
| | | tgaagagctgtacgaagaccgcgcggcaggcgcgtacgacggacccgttggcaggaagcacttccggaagcaa |
| | | caggcagcgctgacgctccggcagcaaggggcggaagagcggcttgccgaacttgaagccgccgaagccccga |
| | | agcttccccttgaccaatggttccccgaagacgccgacgctgacccgaccggccctaagtcgtggtgggggcgcg |
| | | cgtcagtagacgacaagcgcgtgttcgtcgggctcttcgtagacaagatcgttgtcacgaagtcgactacgggca |
| | | gggggcagggaacgcccatcgagaagcgcgcttcgatcacgtgggcgaagccgccgaccgacgacgacgaag |
| | | acgacgcccaggacggcacggaagacgtagcggcgtag |
| 24 | attB | gtgccagggcgtgcccttgggctccccgggcgcg |
| 25 | PE3-CAR | gacggatcgggagatctcccgatgcgggtgccagggcgtgcccttgggctccccgggcgcgtactcctcccctat |
| | plasmid | ggtgcactctcagtacaatctgctctgatgccgcatagttaagccagtatctgctccctgcttgtgtgttggaggtc |
| | | gctgagtagtgcgcgagcaaaatttaagctacaacaaggcaaggcttgaccgacaattgcatgttttaaaagaa |
| | | aaggggggattggggggtacagtgcaggggaaagaatagtagacataatagcaacagacatacaaactaaag |
| | | aattacaaaaacaaattacaaaaattcaaaattttaagaatctgcttagggttaggcgttttgcgctgcttcgcga |
| | | tgtacgggccagatatacgcgttgacattgattattgactagttattaatagtaatcaattacggggtcattagttc |
| | | atagcccatatatggagttccgcgttacataacttacggtaaatggcccgcctggctgaccgcccaacgaccccc |
| | | gcccattgacgtcaataatgacgtatgttcccatagtaacgccaatagggactttccattgacgtcaatgggtgga |
| | | gtatttacggtaaactgcccacttggcagtacatcaagtgtatcatatgccaagtacgccccctattgacgtcaat |
| | | gacggtaaatggcccgcctggcattatgcccagtacatgaccttatgggactttcctacttggcagtacatctacgt |
| | | attagtcatcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttccaaaat |
| | | gtcgtaacaactccgccccattgacgcaaatgggcggtaggcgtgtacggtgggaggtctatataagcagagct |
| | | ctctggctaactagagaacccactgcttactggcttatcgaaattaatacgactcactataaggagacccaagct |
| | | ggctagcgtttaaacttaagcttggtaccgagctcggatccgccgccaccatggccttaccagtgaccgccttgct |
| | | cctgccgctggccttgctgctccacgccgccaggccggatatccagatgacccagacaacctccagcctgtccgc |
| | | ctccctgggcgatagagtgacaatctcctgcagggcctcccaggatatcagcaagtacctgaactggtaccagca |
| | | gaagcccgacggcacagtgaagctgctgatctaccacaccagcaggctgcactccggcgtgccctccaggttca |
| | | gcggctccggaagcggcaccgactacagcctgaccatcagcaatctggagcaggaggatatcgccacctactttt |
| | | gccagcagggcaataccctgccctacaccttcggcggcggcaccaagctggagatcaccggcagcacaagcgg |
| | | ctccggcaagcctggctccggcgaaggaagcacaaagggcgaggtgaagctgcaggagagcggccctggcctg |
| | | gtggctcctagccagtccctgtccgtgacctgtaccgtgtccggcgtgtccctgcctgactacggcgtgagctggat |
| | | caggcagccccccagaaagggcctggagtggctgggcgtgatctggggctccgagaccacatactacaattccg |
| | | ccctgaagagcaggctgaccatcatcaaggataatagcaagtcccaggtgtttctgaagatgaactccctgcag |
| | | accgacgacaccgccatctactactgcgccaagcactactactacggcggcagctacgccatggactactgggg |
| | | ccagggcaccagcgtgaccgtgtcctccggaaaacctatccctaatccactgcttggactcgatagtactaccact |
| | | accccagcaccgaggccacccaccccggctcctaccatcgcctcccagcctctgtccctgcgtccggaggcatgt |
| | | agacccgcagctggtggggccgtgcatacccggggtcttgacttcgcctgcgatttctgggtgctggtcgttgtgg |
| | | gcggcgtgctggcctgctacagcctgctggtgacagtggccttcatcatcttttgggtgaggagcaagcggagca |
| | | gactgctgcacagcgactacatgaacatgaccccccggaggcctggccccacccggaagcactaccagccctac |
| | | gcccctcccagggatttcgccgcctaccggagcaagcgcggtcggaagaagctgctgtacatctttaagcaaccc |
| | | ttcatgaggcctgtgcagactactcaagaggaggacggctgttcatgccggttcccagaggaggaggaaggcgg |
| | | ctgcgaactgcgcgtgaaattcagccgcagcgcagatgctccagcctacaagcaggggcagaaccagctctaca |
| | | acgaactcaatcttggtcggagagaggagtacgacgtgctggacaagcggagaggacgggacccagaaatgg |
| | | gcgggaagccgcgcagaaagaatccccaagagggcctgtacaacgagctccaaaaggataagatggcagaag |
| | | cctatagcgagattggtatgaaaggggaacgcagaagaggcaaaggccacgacggactgtaccagggactca |
| | | gcaccgccaccaaggacacctatgacgctcttcacatgcaggccctgccgcctcggtaatctagagggcccgttt |
| | | aaacccgctgatcagcctcgaaaaaaaaaaaaaaaaaaaaaaaaaaaagaagacggccctgtgccttctagt |
| | | tgccagccatctgttgtttgcccctcccccgtgccttccttgaccctggaaggtgccactcccactgtcctttcctaat |
| | | aaaatgaggaaattgcatcgcattgtctgagtaggtgtcattctattctggggggtggggggggcaggacagca |
| | | agggggaggattgggaagacaatagcaggcatgctggggatgcggtgggctctatggcttctgaggcggaaag |
| | | aaccagctggggctctagggggtatccccggggataacgcaggaaagaacatgtgagcaaaaggccagcaaa |
| | | aggccaggaaccgtaaaaaggccgcgttgctggcgtt |
| 26 | PE3b- | ccttattttaactaagcttaaaaaaattcctgcccgaccttggtaccggatccagtcgacgccatttgtctgcagaa |
| | AAVS1 | ttggcgcacgcgctaaaaacggactagccttattttaacttgctatttctagctctaaaacaaaaaagcaccgact |
| | plasmid | cggtgccactttttcaagttgataacggactagccttattttaacttgctatttctagctctaaaactgccccaactg |
| | | gggtaacctcggtgatggaggtgctgcggtgtttcgtcctttccacaagatatataaagccaagaaatcgaaata |
| | | ctttcaagttacggtaagcatatgatagtccattttaaaacataattttaaaactgcaaactacccaagaaattatt |
| | | actttctacgtcacgtattttgtactaatatctttgtgtttacagtcaaattaattccaattatctctctaacagccttg |
| | | tatcgtatatgcaaatatgaaggaatcatgggaaataggccctcgcgagcaaaatttaagctacaacaaggcaa |
| | | ggcttgaccgacaattgcatgaagaatctgcttagggttaggcgttttgcgctgcttcgcgatgtacgggccagat |
| | | atacgcgtttttaaaagaaaaggggggattggggggtacagtgcaggggaaagaatagtagacataatagcaa |
| | | cagacatacaaactaaagaattacaaaaacaaattacaaaaattcaaaattttctagtccgtttttagggacatt |
| | | gattattgactagttattaatagtaatcaattacggggtcattagttcatagcccatatatggagttccgcgttacat |
| | | aacttacggtaaatggcccgcctggctgaccgcccaacgacccccgcccattgacgtcaataatgacgtatgttc |
| | | ccatagtaacgccaatagggactttccattgacgtcaatgggtggagtatttacggtaaactgcccacttggcagt |
| | | acatcaagtgtatcatatgccaagtacgccccctattgacgtcaatgacggtaaatggcccgcctggcattatgcc |
| | | cagtacatgaccttatgggactttcctacttggcagtacatctacgtattagtcatcgctattaccatggtgatgcg |
| | | gttttggcagtacatcaatgggcgtggatagcggtttgactcacggggatttccaagtctccaccccattgacgtc |
| | | aatgggagtttgttttggcaccaaaatcaacgggactttccaaaatgtcgtaacaactccgccccattgacgcaa |
| | | atgggcggtaggcgtgtacggtgggaggtctatataagcagagctggtttagtgaaccgtcagatccgctagag |
| | | atccgcggccgctaatacgactcactatagggagagccgccaccatgaaacggacagccgacggaagcgagtt |
| | | cgagtcaccaaagaagaagcggaaagtcgacaagaagtacagcatcggcctggacatcggcaccaactctgtg |
| | | ggctgggccgtgatcaccgacgagtacaaggtgcccagcaagaaattcaaggtgctgggcaacaccgaccggc |
| | | acagcatcaagaagaacctgatcggagccctgctgttcgacagcggcgaaacagccgaggccacccggctgaa |
| | | gagaaccgccagaagaagatacaccagacggaagaaccggatctgctatctgcaagagatcttcagcaacgag |
| | | atggccaaggtggacgacagcttcttccacagactggaagagtccttcctggtggaagaggataagaagcacga |
| | | gcggcaccccatcttcggcaacatcgtggacgaggtggcctaccacgagaagtaccccaccatctaccacctga |
| | | gaaagaaactggtggacagcaccgacaaggccgacctgcggctgatctatctggccctggcccacatgatcaag |
| | | ttccggggccacttcctgatcgagggcgacctgaaccccgacaacagcgacgtggacaagctgttcatccagctg |
| | | gtgcagacctacaaccagctgttcgaggaaaaccccatcaacgccagcggcgtggacgccaaggccatcctgtc |
| | | tgccagactgagcaagagcagacggctggaaaatctgatcgcccagctgcccggcgagaagaagaatggcctg |
| | | ttcggaaacctgattgccctgagcctgggcctgacccccaacttcaagagcaacttcgacctggccgaggatgcc |
| | | aaactgcagctgagcaaggacacctacgacgacgacctggacaacctgctggcccagatcggcgaccagtacg |
| | | ccgacctgtttctggccgccaagaacctgtccgacgccatcctgctgagcgacatcctgagagtgaacaccgaga |
| | | tcaccaaggcccccctgagcgcctctatgatcaagagatacgacgagcaccaccaggacctgaccctgctgaaa |
| | | gctctcgtgcggcagcagctgcctgagaagtacaaagagattttcttcgaccagagcaagaacggctacgccgg |
| | | ctacattgacggcggagccagccaggaagagttctacaagttcatcaagcccatcctggaaaagatggacggca |
| | | ccgaggaactgctcgtgaagctgaacagagaggacctgctgcggaagcagcggaccttcgacaacggcagcat |
| | | cccccaccagatccacctgggagagctgcacgccattctgcggcggcaggaagatttttacccattcctgaagga |
| | | caaccgggaaaagatcgagaagatcctgaccttccgcatcccctactacgtgggccctctggccaggggaaaca |
| | | gcagattcgcctggatgaccagaaagagcgaggaaaccatcaccccctggaacttcgaggaagtggtggacaa |
| | | gggcgcttccgcccagagcttcatcgagcggatgaccaacttcgataagaacctgcccaacgagaaggtgctgc |
| | | ccaagcacagcctgctgtacgagtacttcaccgtgtataacgagctgaccaaagtgaaatacgtgaccgaggga |
| | | atgagaaagcccgccttcctgagcggcgagcagaaaaaggccatcgtggacctgctgttcaagaccaaccgga |
| | | aagtgaccgtgaagcagctgaaagaggactacttcaagaaaatcgagtgcttcgactccgtggaaatctccggc |
| | | gtggaagatcggttcaacgcctccctgggcacataccacgatctgctgaaaattatcaaggacaaggacttcctg |
| | | gacaatgaggaaaacgaggacattctggaagatatcgtgctgaccctgacactgtttgaggacagagagatgat |
| | | cgaggaacggctgaaaacctatgcccacctgttcgacgacaaagtgatgaagcagctgaagcggcggagatac |
| | | accggctggggcaggctgagccggaagctgatcaacggcatccgggacaagcagtccggcaagacaatcctgg |
| | | atttcctgaagtccgacggcttcgccaacagaaacttcatgcagctgatccacgacgacagcctgacctttaaag |
| | | aggacatccagaaagcccaggtgtccggccagggcgatagcctgcacgagcacattgccaatctggccggcag |
| | | ccccgccattaagaagggcatcctgcagacagtgaaggtggtggacgagctcgtgaaagtgatgggccggcac |
| | | aagcccgagaacatcgtgatcgaaatggccagagagaaccagaccacccagaagggacagaagaacagccg |
| | | cgagagaatgaagcggatcgaagagggcatcaaagagctgggcagccagatcctgaaagaacaccccgtgga |
| | | aaacacccagctgcagaacgagaagctgtacctgtactacctgcagaatggggggatatgtacgtggaccagg |
| | | aactggacatcaaccggctgtccgactacgatgtggacgctatcgtgcctcagagctttctgaaggacgactcca |
| | | tcgacaacaaggtgctgaccagaagcgacaagaaccggggcaagagcgacaacgtgccctccgaagaggtcg |
| | | tgaagaagatgaagaactactggcggcagctgctgaacgccaagctgattacccagagaaagttcgacaatctg |
| | | accaaggccgagagaggcggcctgagcgaactggataaggccggcttcatcaagagacagctggtggaaaccc |
| | | ggcagatcacaaagcacgtggcacagatcctggactcccggatgaacactaagtacgacgagaatgacaagct |
| | | gatccgggaagtgaaagtgatcaccctgaagtccaagctggtgtccgatttccggaaggatttccagttttacaa |
| | | agtgcgcgagatcaacaactaccaccacgcccacgacgcctacctgaacgccgtcgtgggaaccgccctgatca |
| | | aaaagtaccctaagctggaaagcgagttcgtgtacggcgactacaaggtgtacgacgtgcggaagatgatcgcc |
| | | aagagcgagcaggaaatcggcaaggctaccgccaagtacttcttctacagcaacatcatgaactttttcaagacc |
| | | gagattaccctggccaacggcgagatccggaagcggcctctgatcgagacaaacggcgaaaccggggagatcg |
| | | tgtgggataagggccgggattttgccaccgtgcggaaagtgctgagcatgccccaagtgaatatcgtgaaaaag |
| | | accgaggtgcagacaggcggcttcagcaaagagtctatcctgcccaagaggaacagcgataagctgatcgcca |
| | | gaaagaaggactgggaccctaagaagtacggcggcttcgacagccccaccgtggcctattctgtgctggtggtg |
| | | gccaaagtggaaaagggcaagtccaagaaactgaagagtgtgaaagagctgctggggatcaccatcatggaa |
| | | agaagcagcttcgagaagaatcccatcgactttctggaagccaagggctacaaagaagtgaaaaaggacctga |
| | | tcatcaagctgcctaagtactccctgttcgagctggaaaacggccggaagagaatgctggcctctgccggcgaac |
| | | tgcagaagggaaacgaactggccctgccctccaaatatgtgaacttcctgtacctggccagccactatgagaagc |
| | | tgaagggctcccccgaggataatgagcagaaacagctgtttgtggaacagcacaagcactacctggacgagatc |
| | | atcgagcagatcagcgagttctccaagagagtgatcctggccgacgctaatctggacaaagtgctgtccgcctac |
| | | aacaagcaccgggataagcccatcagagagcaggccgagaatatcatccacctgtttaccctgaccaatctggg |
| | | agcccctgccgccttcaagtactttgacaccaccatcgaccggaagaggtacaccagcaccaaagaggtgctgg |
| | | acgccaccctgatccaccagagcatcaccggcctgtacgagacacggatcgacctgtctcagctgggaggtgac |
| | | tctggaggatctagcggaggatcctctggcagcgagacaccaggaacaagcgagtcagcaacaccagagagc |
| | | agtggcggcagcagcggcggcagcagcaccctaaatatagaagatgagtatcggctacatgagacctcaaaag |
| | | agccagatgtttctctagggtccacatggctgtctgattttcctcaggcctgggcggaaaccgggggcatgggact |
| | | ggcagttcgccaagctcctctgatcatacctctgaaagcaacctctacccccgtgtccataaaacaataccccatg |
| | | tcacaagaagccagactggggatcaagccccacatacagagactgttggaccagggaatactggtaccctgcca |
| | | gtccccctggaacacgcccctgctacccgttaagaaaccagggactaatgattataggcctgtccaggatctgag |
| | | agaagtcaacaagcgggtggaagacatccaccccaccgtgcccaacccttacaacctcttgagcgggctcccac |
| | | cgtcccaccagtggtacactgtgcttgatttaaaggatgcctttttctgcctgagactccaccccaccagtcagcct |
| | | ctcttcgcctttgagtggagagatccagagatgggaatctcaggacaattgacctggaccagactcccacagggt |
| | | ttcaaaaacagtcccaccctgtttaatgaggcactgcacagagacctagcagacttccggatccagcacccagac |
| | | ttgatcctgctacagtacgtggatgacttactgctggccgccacttctgagctagactgccaacaaggtactcggg |
| | | ccctgttacaaaccctagggaacctcgggtatcgggcctcggccaagaaagcccaaatttgccagaaacaggtc |
| | | aagtatctggggtatcttctaaaagagggtcagagatggctgactgaggccagaaaagagactgtgatggggca |
| | | gcctactccgaagacccctcgacaactaagggagttcctagggaaggcaggcttctgtcgcctcttcatccctggg |
| | | tttgcagaaatggcagcccccctgtaccctctcaccaaaccggggactctgtttaattggggcccagaccaacaa |
| | | aaggcctatcaagaaatcaagcaagctcttctaactgccccagccctggggttgccagatttgactaagccctttg |
| | | aactctttgtcgacgagaagcagggctacgccaaaggtgtcctaacgcaaaaactgggaccttggcgtcggccg |
| | | gtggcctacctgtccaaaaagctagacccagtagcagctgggtggcccccttgcctacggatggtagcagccatt |
| | | gccgtactgacaaaggatgcaggcaagctaaccatgggacagccactagtcattctggccccccatgcagtaga |
| | | ggcactagtcaaacaaccccccgaccgctggctttccaacgcccggatgactcactatcaggccttgcttttggac |
| | | acggaccgggtccagttcggaccggtggtagccctgaacccggctacgctgctcccactgcctgaggaagggct |
| | | gcaacacaactgccttgatatcctggccgaagcccacggaacccgacccgacctaacggaccagccgctcccag |
| | | acgccgaccacacctggtacacggatggaagcagtctcttacaagagggacagcgtaaggcgggagctgcggt |
| | | gaccaccgagaccgaggtaatctgggctaaagccctgccagccgggacatccgctcagcgggctgaactgatag |
| | | cactcacccaggccctaaagatggcagaaggtaagaagctaaatgtttatactgatagccgttatgcttttgctac |
| | | tgcccatatccatggagaaatatacagaaggcgtgggtggctcacatcagaaggcaaagagatcaaaaataaa |
| | | gacgagatcttggccctactaaaagccctctttctgcccaaaagacttagcataatccattgtccaggacatcaaa |
| | | agggacacagcgccgaggctagaggcaaccggatggctgaccaagcggcccgaaaggcagccatcacagaga |
| | | ctccagacacctctaccctcctcatagaaaattcatcaccctctggcggctcaaaaagaaccgccgacggcagcg |
| | | aattcgagcccaagaagaagaggaaagtctaatttaaacccgctgatcagcctcgactgtgccttctagttgcca |
| | | gccatctgttgtttgcccctcccccgtgccttccttgaccctggaaggtgccactcccactgtcctttcctaataaaa |
| | | tgagaaaattgcatcgcattgtctgagtaggtgtcattctattctggggggtggggggggcaggacagcaaggg |
| | | ggaggattgggaagacaatagcaggcatgctggggatgcggtgggctctatggcttctgaggcggaaagaacc |
| | | agctggggctcgataccgtcggagggcctatttcccatgattccttcatatttgcatatacgatacaaggctgttag |
| | | agagataattagaattaatttgactgtaaacacaaagatattagtacaaaatacgtgacgtagaaagtaataatt |
| | | tcttgggtagtttgcagttttaaaattatgttttaaaatggactatcatatgcttaccgtaacttgaaagtatttcgat |
| | | ttcttggctttatatatcttgtggaaaggacgaaacaccgagttctgtaattcagcatcaccgcaccccacagtgg |
| | | ggccactgttttagagctagaaatagcaagttaaaataaggctagtccgttatcaacttgaaaaagtggcaccga |
| | | gtcggtgcgatggggcttttctgtcaccaatcctgtccctagtcccccaactgagagaactcaaaggttaccccag |
| | | ttggggcacggccccaacctctagctagagcttggcgtaatcatggtcatagctgtttcctgtgtgaaattgttatc |
| | | cgctcacaattccacacaacatacgagccggaagcataaagtgtaaagcctagggtgcctaatgagtgagctaa |
| | | ctcacattaattgcgttgcgctcactgcccgctttccagtcgggaaacctgtcgtgccagctgcattaatgaatcgg |
| | | ccaacgcgcggggagaggcggtttgcgtattgggcgctcttccgcttcctcgctcactgactcgctgcgctcggtc |
| | | gttcggctgcggcgagcggtatcagctcactcaaaggcggtaatacggttatccacagaatcaggggataacgc |
| | | aggaaagaacatgtgagcaaaaggccagcaaaaggccaggaaccgtaaaaaggccgcgttgctggcgtttttc |
| | | cataggctccgcccccctgacgagcatcacaaaaatcgacgctcaagtcagaggtggcgaaacccgacaggac |
| | | tataaagataccaggcgtttccccctggaagctccctcgtgcgctctcctgttccgaccctgccgcttaccggatac |
| | | ctgtccgcctttctcccttcgggaagcgtggcgctttctcatagctcacgctgtaggtatctcagttcggtgtaggtc |
| | | gttcgctccaagctgggctgtgtgcacgaaccccccgttcagcccgaccgctgcgccttatccggtaactatcgtct |
| | | tgagtccaacccggtaagacacgacttatcgccactggcagcagccactggtaacaggattagcagagcgaggt |
| | | atgtaggcggtgctacagagttcttgaagtggtggcctaactacggctacactagaagaacagtatttggtatctg |
| | | cgctctgctgaagccagttaccttcggaaaaagagttggtagctcttgatccggcaaacaaaccaccgctggtag |
| | | cggtggtttttttgtttgcaagcagcagattacgcgcagaaaaaaaggatctcaagaagatcctttgatcttttcta |
| | | cggggtctgacactcagtggaacgaaaactcacgttaagggattttggtcatgagattatcaaaaaggatcttca |
| | | cctagatccttttaaattaaaaatgaagttttaaatcaatctaaagtatatatgagtaaacttggtctgacagttac |
| | | caatgcttaatcagtgaggcacctatctcagcgatctgtctatttcgttcatccatagttgcctgactccccgtcgtg |
| | | tagataactacgatacgggagggcttaccatctggccccagtgctgcaatgataccgcgagacccacgctcacc |
| | | ggctccagatttatcagcaataaaccagccagccggaagggccgagcgcagaagtggtcctgcaactttatccg |
| | | cctccatccagtctattaattgttgccgggaagctagagtaagtagttcgccagttaatagtttgcgcaacgttgtt |
| | | gccattgctacaggcatcgtggtgtcacgctcgtcgtttggtatggcttcattcagctccggttcccaacgatcaag |
| | | gcgagttacatgatcccccatgttgtgcaaaaaagcggttagctccttcggtcctccgatcgttgtcagaagtaag |
| | | ttggccgcagtgttatcactcatggttatggcagcactgcataattctcttactgtcatgccatccgtaagatgcttt |
| | | tctgtgactggtgagtactcaaccaagtcattctgagaatagtgtatgcggcgaccgagttgctcttgcccggcgt |
| | | caatacgggataataccgcgccacatagcagaactttaaaagtgctcatcattggaaaacgttcttcggggcgaa |
| | | aactctcaaggatcttaccgctgttgagatccagttcgatgtaacccactcgtgcacccaactgatcttcagcatct |
| | | tttactttcaccagcgtttctgggtgagcaaaaacaggaaggcaaaatgccgcaaaaaagggaataagggcga |
| | | cacggaaatgttgaatactcatactcttcctttttcaatattattgaagcatttatcagggttattgtctcatgagcg |
| | | gatacatatttgaatgtatttagaaaaataaacaaataggggttccgcgcacatttccccgaaaagtgccacctg |
| | | acgtcgacggatcgggagatcgatctcccgatcccctagggtctactctcagtacaatctgctctgatgccgcata |
| | | gttaagccagtatctgctccctgcttgtgtgttggaggtcgctgagtagtg |
| 27 | PhiC31 | acggatcgggagatctcccgatcccctatggtgcactctcagtacaatctgctctgatgccgcatagttaagccag |
| | | tatctgctccctgcttgtgtgttggaggtcgctgagtagtgcgcgagcaaaatttaagctacaacaaggcaaggct |
| | | tgaccgacaattgcatgaagaatctgcttagggttaggcgttttgcgctgcttcgcgatgtacgggccagatatac |
| | | gcgttgacattgattattgactagttattaatagtaatcaattacggggtcattagttcatagcccatatatggagtt |
| | | ccgcgttacataacttacggtaaatggcccgcctggctgaccgcccaacgacccccgcccattgacgtcaataat |
| | | gacgtatgttcccatagtaacgccaatagggactttccattgacgtcaatgggtggagtatttacggtaaactgcc |
| | | cacttggcagtacatcaagtgtatcatatgccaagtacgccccctattgacgtcaatgacggtaaatggcccgcct |
| | | ggcattatgcccagtacatgaccttatgggactttcctacttggcagtacatctacgtattagtcatcgctattacca |
| | | tggtgatgcggttttggcagtacatcaatgggcgtggatagcggtttgactcacggggatttccaagtctccaccc |
| | | cattgacgtcaatgggagtttgttttggcaccaaaatcaacgggactttccaaaatgtcgtaacaactccgcccca |
| | | ttgacgcaaatgggcggtaggcgtgtacggtgggaggtctatataagcagagctctctggctaactagagaacc |
| | | cactgcttactggcttatcgaaattaatacgactcactatagggagacccaagcttggtaccgagctcgctagaa |
| | | ataattttgtttaactttaagaaggagatatacatatggacacgtacgcgggtgcttacgaccgtcagtcgcgcga |
| | | gcgcgaaaattcgagcgcagcaagcccagcgacacagcgtagcgccaacgaagacaaggcggccgaccttca |
| | | gcgcgaagtcgagcgcgacgggggccggttcaggttcgtcgggcatttcagcgaagcgccgggcacgtcggcgt |
| | | tcgggacggcggagcgcccggagttcgaacgcatcctgaacgaatgccgcgccgggcggctcaacatgatcatt |
| | | gtctatgacgtgtcgcgcttctcgcgcctgaaggtcatggacgcgattccgattgtctcggaattgctcgccctggg |
| | | cgtgacgattgtttccactcaggaaggcgtcttccggcagggaaacgtcatggacctgattcacctgattatgcgg |
| | | ctcgacgcgtcgcacaaagaatcttcgctgaagtcggcgaagattctcgacacgaagaaccttcagcgcgaatt |
| | | gggcgggtacgtcggcgggaaggcgccttacggcttcgagcttgtttcggagacgaaggagatcacgcgcaacg |
| | | gccgaatggtcaatgtcgtcatcaacaagcttgcgcactcgaccactccccttaccggacccttcgagttcgagcc |
| | | cgacgtaatccggtggtggtggcgtgagatcaagacgcacaaacaccttcccttcaagccgggcagtcaagccg |
| | | ccattcacccgggcagcatcacggggctttgtaagcgcatggacgctgacgccgtgccgacccggggcgagacg |
| | | attgggaagaagaccgcttcaagcgcctgggacccggcaaccgttatgcgaatccttcgggacccgcgtattgcg |
| | | ggcttcgccgctgaggtgatctacaagaagaagccggacggcacgccgaccacgaagattgagggttaccgcat |
| | | tcagcgcgacccgatcacgctccggccggtcgagcttgattgcggaccgatcatcgagcccgctgagtggtatga |
| | | gcttcaggcgtggttggacggcagggggcgcggcaaggggctttcccgggggcaagccattctgtccgccatgg |
| | | acaagctgtactgcgagtgtggcgccgtcatgacttcgaagcgcggggaagaatcgatcaaggactcttaccgct |
| | | gccgtcgccggaaggtggtcgacccgtccgcacctgggcagcacgaaggcacgtgcaacgtcagcatggcggc |
| | | actcgacaagttcgttgcggaacgcatcttcaacaagatcaggcacgccgaaggcgacgaagagacgttggcg |
| | | cttctgtgggaagccgcccgacgcttcggcaagctcactgaggcgcctgagaagagcggcgaacgggcgaacct |
| | | tgttgcggagcgcgccgacgccctgaacgcccttgaagagctgtacgaagaccgcgcggcaggcgcgtacgac |
| | | ggacccgttggcaggaagcacttccggaagcaacaggcagcgctgacgctccggcagcaaggggcggaagag |
| | | cggcttgccgaacttgaagccgccgaagccccgaagcttccccttgaccaatggttccccgaagacgccgacgct |
| | | gacccgaccggccctaagtcgtggtgggggcgcgcgtcagtagacgacaagcgcgtgttcgtcgggctcttcgta |
| | | gacaagatcgttgtcacgaagtcgactacgggcagggggcagggaacgcccatcgagaagcgcgcttcgatca |
| | | cgtgggcgaagccgccgaccgacgacgacgaagacgacgcccaggacggcacggaagacgtagcggcgtagc |
| | | gagacacccggatcgcgccaaaaaaaaaaaaaaaaaaaaaaaaaaaaaaaccggatccactagtaacggcc |
| | | gccagtgtgctggaattctgcagatatccatcacactggcggccgctcgagcatgcatctagagggccctattcta |
| | | tagtgtcacctaaatgctagagctcgctgatcagcctcgactgtgccttctagttgccagccatctgttgtttgcccc |
| | | tcccccgtgccttccttgaccctggaaggtgccactcccactgtcctttcctaataaaatgaggaaattgcatcgca |
| | | ttgtctgagtaggtgtcattctattctggggggtggggtggggcaggacagcaagggggaggattgggaagaca |
| | | atagcaggcatgctggggatgcggtgggctctatggcttctgaggcggaaagaaccagctggggctctaggggg |
| | | tatcccctgtggaattgtctgtataccgtcgacctctagctagagcttggcgtaatcatggtcatagctgtttcctgt |
| | | gggcgtttttccataggctccgcccccctgacgagcatcacaaaaatcgacgctcaagtcagaggtggcgaaac |
| | | ccgacaggactataaagataccaggcgtttccccctggaagctccctcgtgcgctctcctgttccgaccctgccgc |
| | | ttaccggatacctgtccgcctttctcccttcgggaagcgtggcgctttctcatagctcacgctgtaggtatctcagtt |
| | | cggtgtaggtcgttcgctccaagctgggctgtgtgcacgaaccccccgttcagcccgaccgctgcgccttatccgg |
| | | taactatcgtcttgagtccaacccggtaagacacgacttatcgccactggcagcagccactggtaacaggattag |
| | | cagagcgaggtatgtaggcggtgctacagagttcttgaagtggtggcctaactacggctacactagaagaacag |
| | | tatttggtatctgcgctctgctgaagccagttaccttcggaaaaagagttggtagctcttgatccggcaaacaaac |
| | | caccgctggtagcggtttttttgtttgcaagcagcagattacgcgcagaaaaaaaggatctcaagaagatcctttg |
| | | atcttttctacggggtctgacgctcagtggaacgaaaactcacgttaagggattttggtcatgagattatcaaaaa |
| | | ggatcttcacctagatccttttaaattaaaaatgaagttttaaatcaatctaaagtatatatgagtaaacttggtct |
| | | gacagttaccaatgcttaatcagtgaggcacctatctcagcgatctgtctatttcgttcatccatagttgcctgactc |
| | | cccgtcgtgtagataactacgatacgggagggcttaccatctggccccagtgctgcaatgataccgcgagaccca |
| | | cgctcaccggctccagatttatcagcaataaaccagccagccggaagggccgagcgcagaagtggtcctgcaac |
| | | tttatccgcctccatccagtctattaattgttgccgggaagctagagtaagtagttcgccagttaatagtttgcgca |
| | | acgttgttgccattgctacaggcatcgtggtgtcacgctcgtcgtttggtatggcttcattcagctccggttcccaac |
| | | gatcaaggcgagttacatgatcccccatgttgtgcaaaaaagcggttagctccttcggtcctccgatcgttgtcag |
| | | aagtaagttggccgcagtgttatcactcatggttatggcagcactgcataattctcttactgtcatgccatccgtaa |
| | | gatgcttttctgtgactggtgagtactcaaccaagtcattctgagaatagtgtatgcggcgaccgagttgctcttgc |
| | | ccggcgtcaatacgggataataccgcgccacatagcagaactttaaaagtgctcatcattggaaaacgttcttcg |
| | | gggcgaaaactctcaaggatcttaccgctgttgagatccagttcgatgtaacccactcgtgcacccaactgatctt |
| | | cagcatcttttactttcaccagcgtttctgggtgagcaaaaacaggaaggcaaaatgccgcaaaaaagggaata |
| | | agggcgacacggaaatgttgaatactcatactcttcctttttcaatattattgaagcatttatcagggttattgtctc |
| | | atgagcggatacatatttgaatgtatttagaaaaataaacaaataggggttccgcgcacatttccccgaaaagtg |
| | | ccacctgacgt |
| 28 | gRNA TRAC | AGAGUCUCUCAGCUGGUACA |
| 29 | gRNA B2M | GAGUAGCGCGAGCACAGCUA |
| 30 | gRNA PD1 | GACAGCGGCACCUACCUCUG |
| 31 | gRNA CD52 | CAGCCUCCUGGUUAUGGUAC |

AMINO ACID SEQUENCES

| | | |
|---|---|---|
| 21 | p80 RT | LNIEDEHRLHETSKEPDVSLGSTWLSDFPQAWAETGGMGLAVRQAPLIIPLKATSTPVSIK |
| | [Moloney | QYPMSQEARLGIKPHIQRLLDQGILVPCQSPWNTPLLPVKKPGTNDYRPVQDLREVNKRV |
| | murine | EDIHPTVPNPYNLLSGLPPSHQWYTVLDLKDAFFCLRLHPTSQPLFAFEWRDPEMGISGQ |
| | leukemia | LTWTRLPQGFKNSPTLFDEALHRDLADFRIQHPDLILLQYVDDLLLAATSELDCQQGTRALL |
| | virus] | QTLGNLGYRASAKKAQICQKQVKYLGYLLKEGQRWLTEARKETVMGQPTPKTPRQLREFL |
| | NCBI | GTAGFCRLWIPGFAEMAAPLYPLTKTGTLFNWGPDQQKAYQEIKQALLTAPALGLPDLTK |
| | Reference | PFELFVDEKQGYAKGVLTQKLGPWRRPVAYLSKKLDPVAAGWPPCLRMVAAIAVLTKDA |
| | Sequence: | GKLTMGQPLVILAPHAVEALVKQPPDRWLSNARMTHYQALLLDTDRVQFGPVVALNPA |
| | NP_955591.1 | TLLPLPEEGLQHNCLDILAEAHGTRPDLTDQPLPDADHTWYTDGSSLLQEGQRKAGAAVT |
| | | TETEVIWAKALPAGTSAQRAELIALTQALKMAEGKKLNVYTDSRYAFATAHIHGEIYRRRG |
| | | LLTSEGKEIKNKDEILALLKALFLPKRLSIIHCPGHQKGHSAEARGNRMADQAARKAAITET |
| | | PDTSTLLI |
| 22 | Cas9 | DKKYSIGLDIGTNSVGWAVITDEYKVPSKKFKVLGNTDRHSIKKNLIGALLFDSGETAEATRL |
| | (H840A) | KRTARRRYTRRKNRICYLQEIFSNEMAKVDDSFFHRLEESFLVEEDKKHERHPIFGNIVDEV |
| | | AYHEKYPTIYHLRKKLVDSTDKADLRLIYLALAHMIKFRGHFLIEGDLNPDNSDVDKLFIQLV |
| | | QTYNQLFEENPINASGVDAKAILSARLSKSRRLENLIAQLPGEKKNGLFGNLIALSLGLTPNF |
| | | KSNFDLAEDAKLQLSKDTYDDDLDNLLAQIGDQYADLFLAAKNLSDAILLSDILRVNTEITK |
| | | APLSASMIKRYDEHHQDLTLLKALVRQQLPEKYKEIFFDQSKNGYAGYIDGGASQEEFYKFI |
| | | KPILEKMDGTEELLVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGASAQSFIERMTNFD |
| | | KNLPNEKVLPKHSLLYEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNEENEDILEDIVLTLTLF |
| | | EDREMIEERLKTYAHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKKGILQTVKVVDELV |
| | | KVMGRHKPENIVIEMARENQTTQKGQKNSRERMKRIEEGIKELGSQILKEHPVENTQLQ |
| | | NEKLYLYYLQNGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKRQLVETRQI |
| | | TKHVAQILDSRMNTKYDENDKLIREVKVITLKSKLVSDFRKDFQFYKVREINNYHHAHDAY |
| | | LNAVVGTALIKKYPKLESEFVYGDYKVYDVRKMIAKSEQEIGKATAKYFFYSNIMNFFKTEIT |
| | | LANGEIRKRPLIETNGETGEIVWDKGRDFATVRKVLSMPQVNIVKKTEVQTGGFSKESILP |
| | | KRNSDKLIARKKDWDPKKYGGFDSPTVAYSVLVVAKVEKGKSKKLKSVKELLGITIMERSSF |
| | | EKNPIDFLEAKGYKEVKKDLIIKLPKYSLFELENGRKRMLASAGELQKGNELALPSKYVNFLY |
| | | LASHYEKLKGSPEDNEQKQLFVEQHKHYLDEIIEQISEFSKRVILADANLDKVLSAYNKHRD |
| | | KPIREQAENIIHLFTLTNLGAPAAFKYFDTTIDRKRYTSTKEVLDATLIHQSITGLYETRIDLSQ |
| | | LGGDSGGSSGGSSDLSQLGGD |
| 23 | phage phi- | MDTYAGAYDRQSRERENSSAASPATQRSANEDKAADLQREVERDGGRFRFVGHFSEAP |
| | C31 | GTSAFGTAERPEFERILNECRAGRLNMIIVYDVSRFSRLKVMDAIPIVSELLALGVTIVSTQE |
| | integrase | GVFRQGNVMDLIHLIMRLDASHKESSLKSAKILDTKNLQRELGGYVGGKAPYGFELVSETK |
| | | EITRNGRMVNVVINKLAHSTTPLTGPFEFEPDVIRWWWREIKTHKHLPFKPGSQAAIHPG |
| | | SITGLCKRMDADAVPTRGETIGKKTASSAWDPATVMRILRDPRIAGFAAEVIYKKKPDGTP |
| | | TTKIEGYRIQRDPITLRPVELDCGPIIEPAEWYELQAWLDGRGRGKGLSRGQAILSAMDKL |
| | | YCECGAVMTSKRGEESIKDSYRCRRRKVVDPSAPGQHEGTCNVSMAALDKFVAERIFNKI |
| | | RHAEGDEETLALLWEAARRFGKLTEAPEKSGERANLVAERADALNALEELYEDRAAGAYD |
| | | GPVGRKHFRKQQAALTLRQQGAEERLAELEAAEAPKLPLDQWFPEDADADPTGPKSW |
| | | WGRASVDDKRVFVGLFVDKIVVTKSTTGRGQGTPIEKRASITWAKPPTDDDEDDAQDGT |
| | | EDVAA |

1. A method for modifying a cell, the method comprising:
contacting the cell with a first set of components of a genome editing system, wherein the first set of components comprises a guide RNA having a nucleotide sequence capable of hybridizing to a target location, a pegRNA having an attP attachment site, and a nickase or a nucleic acid having a nucleotide sequence encoding the nickase; and
contacting the cell with a second set and a third set of components of a genome editing system, wherein the second set of components comprises a serine-integrase (PhiC31) or a nucleic acid having a nucleotide sequence encoding a serine-integrase (PhiC31), and the third set of components comprises a nucleotide sequence encoding a nucleic acid sequence of interest and an attB site docking site.

2. The method of claim 1, wherein contacting the cell with a second set of components and a third set of components results in a co-transfection event.

3. The method of claim 1, wherein modifying the cell is an insertion of the nucleic acid sequence of interest into the genome of the cell.

4. The method of claim 3, wherein the insertion is a nucleic acid sequence encoding a chimeric antigen receptor (CAR).

5. The method of claim 4, wherein forming a CAR-T cell or a CAR-NK cell does not comprise use of a viral vector.

6. The method of claim 1, wherein the nucleic acid sequence of interest is inserted in absence of homologous recombination.

7. The method of claim 1, wherein delivering the first set, second set and third set of genomic editing components to the cell does not comprise use of a viral vector.

8. The method of claim 1, wherein the contacting the cell with the first, second and third sets of genome editing components comprises combining the cell in fluid medium with the first, second and third sets of genome editing components and passing the fluid medium through at least one microfluidic transfection device.

9. The method of claim 8, wherein a first vector comprises the first set of first set of genomic editing components, a second vector comprises the second set of genomic editing components, and a third vector comprises the third set of genome editing components.

10. The method of claim 9, wherein the first, second and third vectors are delivered to the cell by passing the cell in fluid medium through one or more microfluidic transfection devices.

11. The method of claim 9, wherein the first vector comprising a nickase comprises a nucleotide sequence having at least 70%, 80%, 90%, or 100% sequence identity with SEQ ID NO: 18 (nCas9).

12. The method of any one of claim 1, wherein the nickase comprises a Cas9 nickase.

13. The method of any one of claim 1, wherein the nickase comprises nCas9 having a nucleic acid sequence having at least 70%, 80%, 90% or 100% sequence identity with SEQ ID NO:18.

14. The method of any one of claim 1 wherein the nickase comprises an nCas9-M-MLV fusion protein with M-MLV having an amino acid sequence having at least 70%, 80%, 90% or 100% sequence identity with SEQ ID NO: 21.

15. The method of claim 1, wherein the cell is selected from an immune cell, a neural cell and a stem cell.

16. The method of claim 1, wherein the cell is a resting immune cell.

17. The method of claim 1, wherein the cell is a resting T cell.

18. The method of claim 1, wherein the cell is a resting NK cell.

19. The method of claim 1, wherein the cell is a naïve stem cell, or a pluripotent stem cell.

20. The method of claim 1, wherein the cell is a T cell or an NK cell and the method further comprises maintaining the cell for a time and under conditions sufficient for the cell to form a CAR-T cell or a CAR-NK cell.
